(19) United States
(12) Patent Application Publication
(10) Pub. No.: US 2023/0351586 A1
(43) Pub. Date: Nov. 2, 2023

(54) SYSTEMS AND METHODS FOR ARTIFICIAL INTELLIGENCE-BASED IMAGE ANALYSIS FOR DETECTION AND CHARACTERIZATION OF LESIONS

(71) Applicant: EXINI Diagnostics AB, Lund (SE)

(72) Inventors: Johan Martin Brynolfsson, Helsingborg (SE); Kerstin Elsa Maria Johnsson, Lund (SE); Hannicka Maria Eleonora Sahlstedt, Malmö (SE); Jens Filip Andreas Richter, Lund (SE)

(21) Appl. No.: 18/014,214

(22) PCT Filed: Jul. 2, 2021
(86) PCT No.: PCT/EP2021/068337

§ 371 Date: Jan. 3, 2023

Related U.S. Application Data
Application No. PCT/EP2021/068337 is a continuation-in-part of application No. 17/008,411, filed on Aug. 31, 2020.
(60) Provisional application No. 63/209,317, filed on Jun. 10, 2021.
(60) Provisional application No. 63/127,666, filed on Dec. 18, 2020.
(60) Provisional application No. 63/048,436, filed on Jul. 6, 2020.

(51) Int. Cl. G06T 15/00; G06T 7/00; G06V 10/25; G16H 50/30; G06T 7/10; G06V 10/764

(52) U.S. Cl. CPC G06T 7/0012; G06T 7/10; G06T 15/00; G06V 10/25; G06V 10/764; G16H 50/30; G06T 2207/10072; G06T 2207/30056; G06T 2207/30084; G06T 2207/30096; G06V 2201/07

(57) ABSTRACT

Presented herein are systems and methods that provide for improved detection and characterization of lesions within a subject via automated analysis of nuclear medicine images, such as positron emission tomography (PET) and single photon emission computed tomography (SPECT) images. In particular, in certain embodiments, the approaches described herein leverage artificial intelligence (AI) to detect regions of 3D nuclear medicine images corresponding to hotspots that represent potential cancerous lesions in the subject. The machine learning modules may be used not only to detect presence and locations of such regions within an image, but also to segment the region corresponding to the lesion and/or classify such hotspots based on the likelihood that they are indicative of a true, underlying cancerous lesion. This AI-based lesion detection, segmentation, and classification can provide a basis for further characterization of lesions, overall tumor burden, and estimation of disease severity and risk.

### CROSS REFERENCE TO RELATED APPLICATIONS

This application claims priority to and benefit of U.S. Provisional Pat. Application No. 63/048,436, filed Jul. 6, 2020, U.S. Non-Provisional Pat. Application No. 17/008,411, filed Aug. 31, 2020, U.S. Provisional Pat. Application No. 63/127,666, filed Dec. 18, 2020, and U.S. Provisional Pat. Application No. 63/209,317, filed Jun. 10, 2021, the contents of each of which are hereby incorporated by reference in their entirety.

### TECHNICAL FIELD

This invention relates generally to systems and methods for creation, analysis, and/or presentation of medical image data. More particularly, in certain embodiments, the invention relates to systems and methods for automated analysis of medical images to identify and/or characterize cancerous lesions.

### BACKGROUND

Nuclear medicine imaging involves the use of radiolabeled compounds, referred to as radiopharmaceuticals. Radiopharmaceuticals are administered to patients and accumulate in various regions in the body in manner that depends on, and is therefore indicative of, biophysical and/or biochemical properties of tissue therein, such as those influenced by presence and/or state of disease, such as cancer. For example, certain radiopharmaceuticals, following administration to a patient, accumulate in regions of abnormal osteogenesis associated with malignant bone lesions, which are indicative of metastases. Other radiopharmaceuticals may bind to specific receptors, enzymes, and proteins in the body that are altered during evolution of disease. After administration to a patient, these molecules circulate in the blood until they find their intended target. The bound radiopharmaceutical remains at the site of disease, while the rest of the agent clears from the body.

Nuclear medicine imaging techniques capture images by detecting radiation emitted from the radioactive portion of the radiopharmaceutical. The accumulated radiopharmaceutical serves as a beacon so that an image may be obtained depicting the disease location and concentration using commonly available nuclear medicine modalities. Examples of nuclear medicine imaging modalities include bone scan imaging (also referred to as scintigraphy), single-photon emission computerized tomography (SPECT), and positron emission tomography (PET). Bone scan, SPECT, and PET imaging systems are found in most hospitals throughout the world. Choice of a particular imaging modality depends on and/or dictates the particular radiopharmaceutical used. For example, technetium 99m (^{99m}Tc) labeled compounds are compatible with bone scan imaging and SPECT imaging, while PET imaging often uses fluorinated compounds labeled with 18F. The compound ^{99m}Tc methylenediphosphonate (^{99m}Tc MDP) is a popular radiopharmaceutical used for bone scan imaging in order to detect metastatic cancer. Radiolabeled prostate-specific membrane antigen (PSMA) targeting compounds such as ^{99m}Tc labeled 1404 and PyL™ (also referred to as [18F]DCFPyL) can be used with SPECT and PET imaging, respectively, and offer the potential for highly specific prostate cancer detection.

Accordingly, nuclear medicine imaging is a valuable technique for providing physicians with information that can be used to determine the presence and the extent of disease in a patient. The physician can use this information to provide a recommended course of treatment to the patient and to track the progression of disease.

For example, an oncologist may use nuclear medicine images from a study of a patient as input in her assessment of whether the patient has a particular disease, e.g., prostate cancer, what stage of the disease is evident, what the recommended course of treatment (if any) would be, whether surgical intervention is indicated, and likely prognosis. The oncologist may use a radiologist report in this assessment. A radiologist report is a technical evaluation of the nuclear medicine images prepared by a radiologist for a physician who requested the imaging study and includes, for example, the type of study performed, the clinical history, a comparison between images, the technique used to perform the study, the radiologist’s observations and findings, as well as overall impressions and recommendations the radiologist may have based on the imaging study results. A signed radiologist report is sent to the physician ordering the study for the physician’s review, followed by a discussion between the physician and patient about the results and recommendations for treatment.

Thus, the process involves having a radiologist perform an imaging study on the patient, analyzing the images obtained, creating a radiologist report, forwarding the report to the requesting physician, having the physician formulate an assessment and treatment recommendation, and having the physician communicate the results, recommendations, and risks to the patient. The process may also involve repeating the imaging study due to inconclusive results, or ordering further tests based on initial results. If an imaging study shows that the patient has a particular disease or condition (e.g., cancer), the physician discusses various treatment options, including surgery, as well as risks of doing nothing or adopting a watchful waiting or active surveillance approach, rather than having surgery.

Accordingly, the process of reviewing and analyzing multiple patient images, over time, plays a critical role in the diagnosis and treatment of cancer. There is a significant need for improved tools that facilitate and improve accuracy of image review and analysis for cancer diagnosis and treatment. Improving the toolkit utilized by physicians, radiologists, and other healthcare professionals in this manner provides for significant improvements in standard of care and patient experience.

### SUMMARY OF THE INVENTION

Presented herein are systems and methods that provide for improved detection and characterization of lesions within a subject via automated analysis of nuclear medicine images, such as positron emission tomography (PET) and single photon emission computed tomography (SPECT) images. In particular, in certain embodiments, the approaches described herein leverage artificial intelligence (AI) techniques to detect regions of 3D nuclear medicine images that represent potential cancerous lesions in the subject. In certain embodiments, these regions correspond to localized regions of elevated intensity relative to their surroundings - hotspots - due to increased uptake of radiopharmaceutical within lesions. The systems and methods described herein may use one or more machine learning modules not only to detect presence and locations of such hotspots within an image, but also to segment the region corresponding to the hotspot and/or classify hotspots based on the likelihood that they indeed correspond to a true, underlying cancerous lesion. These AI-based lesion detection, segmentation, and classification approaches can provide a basis for further characterization of lesions, overall tumor burden, and estimation of disease severity and risk.

For example, once image hotspots representing lesions are detected, segmented, and classified, lesion index values can be computed to provide a measure of radiopharmaceutical uptake within and/or a size (e.g., volume) of the underlying lesion. The computed lesion index values can, in turn, be aggregated to provide an overall estimate of tumor burden, disease severity, metastasis risk, and the like, for the subject. In certain embodiments, lesion index values are computed by comparing measures of intensities within segmented hotspot volumes to intensities of specific reference organs, such as liver and aorta portions. Using reference organs in this manner allows for lesion index values to be measured on a normalized scale that can be compared between images of different subjects. In certain embodiments, the approaches described herein include techniques for suppressing intensity bleed from multiple image regions that correspond to organs and tissue regions in which radiopharmaceutical accumulates at high-levels under normal circumstances, such as a kidney, liver, and a bladder (e.g., urinary bladder). Intensities in regions of nuclear medicine images corresponding to these organs are typically high even for normal, healthy subjects, and not necessarily indicative of cancer. Moreover, high radiopharmaceutical accumulation in these organs results in high levels of emitted radiation. The increased emitted radiation can scatter, resulting not just in high intensities within regions of nuclear medicine images corresponding to the organs themselves, but also at nearby outside voxels. This intensity bleed, into regions of an image outside and around regions corresponding to an organ associated with high uptake, can hinder detection of nearby lesions and cause inaccuracies in measuring uptake therein. Accordingly, correcting such intensity bleed effects improves accuracy of lesion detection and quantification.

In certain embodiments, the AI-based lesion detection technique described herein augment the functional information obtained from nuclear medicine images with anatomical information obtained from anatomical images, such as x-ray computed tomography (CT) images. For example, machine learning modules utilized in the approaches described herein may receive multiple channels of input, including a first channel corresponding to a portion of a functional, nuclear medicine, image (e.g., a PET image; e.g., a SPECT image), as well as additional channels corresponding to a portion of a co-aligned anatomical (e.g., CT) image and/or anatomical information derived therefrom. Adding anatomical context in this manner may improve accuracy of lesion detection approaches. Anatomical information may also be incorporated into lesion classification approaches applied following detection. For example, in addition to computing lesion index values based on intensities of detected hotspots, hotspots may also be assigned an anatomical label based on their location. For example, detected hotspots may be automatically assigned an label (e.g., an alphanumeric label) based on whether their locations correspond to locations within a prostate, pelvic lymph node, non-pelvic lymph node, bone, or a soft-tissue region outside the prostate and lymph nodes.

In certain embodiments, detected hotspots and associated information, such as computed lesion index values and anatomical labeling, are displayed with an interactive graphical user interface (GUI) so as to allow for review by a medical professional, such as a physician, radiologist, technician, etc. Medical professionals may thus use the GUI to review and confirm accuracy of detected hotspots, as well as corresponding index values and/or anatomical labeling. In certain embodiments, the GUI may also allow users to identify and segment (e.g., manually) additional hotspots within medical images, thereby allowing a medical professional to identify additional potential lesions that he/she believes the automated detection process may have missed. Once identified, lesion index values and/or anatomical labeling may also be determined for these manually identified and segmented lesions. Once a user is satisfied with the set of detected hotspots and information computed therefrom, they may confirm their approval and generate a final, signed, report that can, for example, be reviewed and used to discuss outcomes and diagnosis with a patient, and assess prognosis and treatment options.

In this manner, the approaches described herein provide AI-based tools for lesion detection and analysis that can improve accuracy of and streamline assessment of disease (e.g., cancer) state and progression in a subject. This facilitates diagnosis, prognosis, and assessment of response to treatment, thereby improving patient outcomes.

In one aspect, the invention is directed to a method for automatically processing 3D images of a subject to identify and/or characterize (e.g., grade) cancerous lesions within the subject, the method comprising: (a) receiving (e.g., and/or accessing), by a processor of a computing device, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value (e.g., standard uptake value (SUV)) that represents detected radiation emitted from the particular physical volume, wherein at least a portion of the plurality of voxels of the 3D functional image represent physical volumes within the target tissue region]; (b) automatically detecting, by the processor, using a machine learning module [e.g., a pre-trained machine learning module (e.g., having pre-determined (e.g., and fixed) parameters having been determined via a training procedure)], one or more hotspots within the 3D functional image, each hotspot corresponding to a local region of elevated intensity with respect to its surrounding and representing (e.g., indicative of) a potential cancerous lesion within the subject, thereby creating one or both of (i) and (ii) as follows: (i) a hotspot list [e.g., a list of coordinates (e.g., image coordinates; e.g., physical space coordinates); e.g., a mask identifying voxels of the 3D functional image corresponding to a location (e.g., a center of mass) of a detected hotspot] identifying, for each hotspot, a location of the hotspot, and (ii) a 3D hotspot map, identifying, for each hotspot, a corresponding 3D hotspot volume within the 3D functional image {e.g., wherein, the 3D hotspot map is a segmentation map (e.g., comprising one or more segmentation masks) identifying, for each hotspot, voxels within the 3D functional image corresponding to the 3D hotspot volume of each hotspot [e.g., wherein the 3D hotspot map is obtained via artificial intelligence-based segmentation of the functional image (e.g., using a machine-learning module that receives, as input, at least the 3D functional image and generates the 3D hotspot map as output, thereby segmenting hotspots)]; e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)}; and (c) storing and/or providing, for display and/or further processing, the hotspot list and/or the 3D hotspot map.

In certain embodiments, the machine learning module receives, as input, at least a portion of the 3D functional image and automatically detects the one or more hotspots based at least in part on intensities of voxels of the received portion of the 3D functional image. In certain embodiments, the machine learning module receives, as input, a 3D segmentation map that identifies one or more volumes of interest (VOIs) within the 3D functional image, each VOI corresponding to a particular target tissue region and/or a particular anatomical region within the subject [e.g., a soft-tissue region (e.g., a prostate, a lymph node, a lung, a breast); e.g., one or more particular bones; e.g., an overall skeletal region].

In certain embodiments, the method comprises receiving (e.g., and/or accessing), by the processor, a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject, and the machine learning module receives at least two channels of input, said input channels comprising a first input channel corresponding to at least a portion of the 3D anatomical image and a second input channel corresponding to at least a portion of the 3D functional image [e.g., wherein the machine learning module receives a PET image and a CT image as separate channels (e.g., separate channels representative of the same volume) (e.g., analogous to receipt by a machine learning module of two color channels (RGB) of a photographic color image)].

In certain embodiments, the machine learning module receives, as input, a 3D segmentation map that identifies, within the 3D functional image and/or the 3D anatomical image, one or more volumes of interest (VOIs), each VOI corresponding to a particular target tissue region and/or a particular anatomical region. In certain embodiments, the method comprises automatically segmenting, by the processor, the 3D anatomical image, thereby creating the 3D segmentation map.

In certain embodiments, the machine learning module is a region-specific machine learning module that receives, as input, a specific portion of the 3D functional image corresponding to one or more specific tissue regions and/or anatomical regions of the subject.

In certain embodiments, the machine learning module generates, as output, the hotspot list [e.g., wherein the machine learning module implements a machine learning algorithm (e.g., an artificial neural network (ANN)) trained to determine, based on intensities of voxels of at least a portion of the 3D functional image, one or more locations (e.g., 3D coordinates), each corresponding to a location of one of the one or more hotspots].

In certain embodiments, the machine learning module generates, as output, the 3D hotspot map [e.g., wherein the machine learning module implements a machine learning algorithm (e.g., an artificial neural network (ANN)) trained to segment the 3D functional image (e.g., based at least in part on intensities of voxels of the 3D functional image) to identify the 3D hotspot volumes of the 3D hotspot map (e.g., the 3D hotspot map delineating, for each hotspot, a 3D boundary (e.g., an irregular boundary) of the hotspot, thereby identifying the 3D hotspot volumes (e.g., enclosed by the 3D hotspot boundaries)); e.g., wherein the machine learning module implements a machine learning algorithm trained to determine, for each voxel of at least a portion of the 3D functional image, a hotspot likelihood value representing a likelihood that the voxel corresponds to a hotspot (e.g., and step (b) comprises performing one or more subsequent post-processing steps, such as thresholding, to identify the 3D hotspot volumes of the 3D hotspot map using the hotspot likelihood values (e.g., the 3D hotspot map delineating, for each hotspot, a 3D boundary (e.g., an irregular boundary) of the hotspot, thereby identifying the 3D hotspot volumes (e.g., enclosed by the 3D hotspot boundaries)))].

In certain embodiments, the method comprises: (d) determining, by the processor, for each hotspot of at least a portion of the hotspots, a lesion likelihood classification corresponding to a likelihood of the hotspot representing a lesion within the subject [e.g., a binary classification indicative of whether the hotspot is a true lesion or not; e.g., a likelihood value on a scale (e.g., a floating point value ranging from zero to one) representing a likelihood of the hotspot representing a true lesion].

In certain embodiments, step (d) comprises using a second machine learning module to determine, for each hotspot of the portion, the lesion likelihood classification [e.g., wherein the machine learning module implements a machine learning algorithm trained to detect hotspots (e.g., to generate, as output, the hotspot list and/or the 3D hotspot map) and to determine, for each hotspot, the lesion likelihood classification for the hotpot]. In certain embodiments, step (d) comprises using a second machine learning module (e.g., a hotspot classification module) to determine the lesion likelihood classification for each hotspot [e.g., based at least in part on one or more members selected from the group consisting of: intensities of the 3D functional image, the hotspot list, the 3D hotspot map, intensities of a 3D anatomical image, and a 3D segmentation map; e.g., wherein the second machine learning module receives one or more channels of input corresponding to one or more members selected from the group consisting of intensities of the 3D functional image, the hotspot list, the 3D hotspot map, intensities of a 3D anatomical image, and a 3D segmentation map].

In certain embodiments, the method comprises determining, by the processor, for each hotspot, a set of one or more hotspot features and using the set of the one or more hotspot features as input to the second machine learning module.

In certain embodiments, the method comprises: (e) selecting, by the processor, based at least in part on the lesion likelihood classifications for the hotspots, a subset of the one or more hotspots corresponding to hotspots having a high likelihood of corresponding to cancerous lesions (e.g., for inclusion in a report; e.g., for use in computing one or more risk index values for the subject).

In certain embodiments, the method comprises: (f) [e.g., prior to step (b)] adjusting intensities of voxels of the 3D functional image, by the processor, to correct for intensity bleed (e.g., cross-talk) from one or more high-intensity volumes of the 3D functional image, each of the one or more high-intensity volumes corresponding to a high-uptake tissue region within the subject associated with high radiopharmaceutical uptake under normal circumstances (e.g., not necessarily indicative of cancer). In certain embodiments, step (f) comprises correcting for intensity bleed from a plurality of high-intensity volumes one at a time, in a sequential fashion [e.g., first adjusting intensities of voxels of the 3D functional image to correct for intensity bleed from a first high-intensity volume to generate a first corrected image, then adjusting intensities of voxels of the first corrected image to correct for intensity bleed from a second high-intensity volume, and so on]. In certain embodiments, the one or more high-intensity volumes correspond to one or more high-uptake tissue regions selected from the group consisting of a kidney, a liver, and a bladder (e.g., a urinary bladder).

In certain embodiments, the method comprises: (g) determining, by the processor, for each of at least a portion of the one or more hotspots, a corresponding lesion index indicative of a level of radiopharmaceutical uptake within and/or size (e.g., volume) of an underlying lesion to which the hotspot corresponds. In certain embodiments, step (g) comprises comparing an intensity (intensities) (e.g., corresponding to standard uptake values (SUVs)) of one or more voxels associated with the hotspot (e.g., at and/or about a location of the hotspot; e.g., within a volume of the hotspot) with one or more reference values, each reference value associated with a particular reference tissue region (e.g., a liver; e.g., an aorta portion) within the subject and determined based on intensities (e.g., SUV values) of a reference volume corresponding to the reference tissue region [e.g., as an average (e.g., a robust average, such as a mean of values in an interquartile range)]. In certain embodiments, the one or more reference values comprise one or more members selected from the group consisting of an aorta reference value associated with an aorta portion of the subject and a liver reference value associated with a liver of the subject.

In certain embodiments, for at least one particular reference value associated with a particular reference tissue region, determining the particular reference value comprises fitting intensities of voxels [e.g., fitting an distribution of intensities of voxels (e.g., fitting a histogram of voxel intensities)] within a particular reference volume corresponding to the particular reference tissue region to a multi-component mixture model (e.g., a two-component Gaussian model)[e.g., and identifying one or more minor peaks in a distribution of voxel intensities, said minor peaks corresponding to voxels associated with anomalous uptake, and excluding those voxels from the reference value determination (e.g., thereby accounting for effects of abnormally low radiopharmaceutical uptake in certain portions of reference tissue regions, such as portions of the liver)].

In certain embodiments, the method comprises using the determined lesion index values compute (e.g., automatically, by the processor) an overall risk index for the subject, indicative of a caner status and/or risk for the subject.

In certain embodiments, the method comprises determining, by the processor (e.g., automatically), for each hotspot, an anatomical classification corresponding to a particular anatomical region and/or group of anatomical regions within the subject in which the potential cancerous lesion that the hotspot represents is determined [e.g., by the processor (e.g., based on a received and/or determined 3D segmentation map)] to be located [e.g., within a prostate, a pelvic lymph node, a non-pelvic lymph node, a bone (e.g., a bone metastatic region), and a soft tissue region not situated in prostate or lymph node].

In certain embodiments, the method comprise: (h) causing, by the processor, for display within a graphical user interface (GUI), graphical representation of at least a portion of the one or more hotspots for review by a user. In certain embodiments, the method comprises: (i) receiving, by the processor, via the GUI, a user selection of a subset of the one or more hotspots confirmed via user review as likely to represent underlying cancerous lesions within the subject.

In certain embodiments, the 3D functional image comprises a PET or SPECT image obtained following administration of an agent (e.g., a radiopharmaceutical; e.g., an imaging agent) to the subject. In certain embodiments, the agent comprises a PSMA binding agent. In certain embodiments, the agent comprises ¹⁸F. In certain embodiments, the agent comprises [18F]DCFPyL. In certain embodiments, the agent comprises the agent comprises PSMA-11 (e.g., ⁶⁸Ga-PSMA-11). In certain embodiments, the agent comprises one or more members selected from the group consisting of ^{99mc}Tc, ⁶⁸Ga, ¹⁷⁷Lu, ²²⁵Ac, ¹¹¹In, ¹²³I, ¹²⁴I, and ¹³¹I.

In certain embodiments, the machine learning module implements a neural network [e.g., an artificial neural network (ANN); e.g., a convolutional neural network (CNN)].

In certain embodiments, the processor is a processor of a cloud-based system.

In another aspect, the invention is directed to a method for automatically processing 3D images of a subject to identify and/or characterize (e.g., grade) cancerous lesions within the subject, the method comprising: (a) receiving (e.g., and/or accessing), by a processor of a computing device, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) receiving (e.g., and/or accessing), by the processor, a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject; (c) automatically detecting, by the processor, using a machine learning module, one or more hotspots within the 3D functional image, each hotspot corresponding to a local region of elevated intensity with respect to its surrounding and representing (e.g., indicative of) a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image {e.g., wherein, the 3D hotspot map is a segmentation map (e.g., comprising one or more segmentation masks) identifying, for each hotspot, voxels within the 3D functional image corresponding to the 3D hotspot volume of each hotspot [e.g., wherein the 3D hotspot map is obtained via artificial intelligence-based segmentation of the functional image (e.g., using a machine-learning module that receives, as input, at least the 3D functional image and generates the 3D hotspot map as output, thereby segmenting hotspots)]; e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)}, wherein the machine learning module receives at least two channels of input, said input channels comprising a first input channel corresponding to at least a portion of the 3D anatomical image and a second input channel corresponding to at least a portion of the 3D functional image [e.g., wherein the machine learning module receives a PET image and a CT image as separate channels (e.g., separate channels representative of the same volume) (e.g., analogous to receipt by a machine learning module of two color channels (RGB) of a photographic color image)] and/or anatomical information derived therefrom [e.g., a 3D segmentation map that identifies, within the 3D functional image, one or more volumes of interest (VOIs), each VOI corresponding to a particular target tissue region and/or a particular anatomical region]; and (d) storing and/or providing for display and/or further processing, the hotspot list and/or the 3D hotspot map.

In another aspect, the invention is directed to a method for automatically processing 3D images of a subject to identify and/or characterize (e.g., grade) cancerous lesions within the subject, the method comprising: (a) receiving (e.g., and/or accessing), by a processor of a computing device, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) automatically detecting, by the processor, using a first machine learning module, one or more hotspots within the 3D functional image, each hotspot corresponding to a local region of elevated intensity with respect to its surrounding and representing (e.g., indicative of) a potential cancerous lesion within the subject, thereby creating a hotspot list identifying, for each hotspot, a location of the hotspot [e.g., wherein the machine learning module implements a machine learning algorithm (e.g., an artificial neural network (ANN)) trained to determine, based on intensities of voxels of at least a portion of the 3D functional image, one or more locations (e.g., 3D coordinates), each corresponding to a location of one of the one or more hotspots]; (c) automatically determining, by the processor, using a second machine learning module and the hotspot list, for each of the one or more hotspots, a corresponding 3D hotspot volume within the 3D functional image, thereby creating a 3D hotspot map [e.g., wherein the second machine learning module implements a machine learning algorithm (e.g., an artificial neural network (ANN)) trained to segment the 3D functional image based at least in part on the hotspot list along with intensities of voxels of the 3D functional image to identify the 3D hotspot volumes of the 3D hotspot map; e.g., wherein the machine learning module implements a machine learning algorithm trained to determine, for each voxel of at least a portion of the 3D functional image, a hotspot likelihood value representing a likelihood that the voxel corresponds to a hotspot (e.g., and step (b) comprises performing one or more subsequent post-processing steps, such as thresholding, to identify the 3D hotspot volumes of the 3D hotspot map using the hotspot likelihood values][e.g., wherein, the 3D hotspot map is a segmentation map (e.g., comprising one or more segmentation masks) generated using (e.g., based on and/or corresponding to output from) the second machine learning module, the 3D hotspot map identifying, for each hotspot, voxels within the 3D functional image corresponding to the 3D hotspot volume of each hotspot); e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)]; and (d) storing and/or providing for display and/or further processing, the hotspot list and/or the 3D hotspot map.

In certain embodiments, the method comprises: (e) determining, by the processor, for each hotspot of at least a portion of the hotspots, a lesion likelihood classification corresponding to a likelihood of the hotspot representing a lesion within the subject. In certain embodiments, step (e) comprises using a third machine learning module (e.g., a hotspot classification module) to determine the lesion likelihood classification for each hotspot [e.g., based at least in part on one or more members selected from the group consisting of intensities of the 3D functional image, the hotspot list, the 3D hotspot map, intensities of a 3D anatomical image, and a 3D segmentation map; e.g., wherein the third machine learning module receives one or more channels of input corresponding to one or more members selected from the group consisting of intensities of the 3D functional image, the hotspot list, the 3D hotspot map, intensities of a 3D anatomical image, and a 3D segmentation map].

In certain embodiments, the method comprises: (f) selecting, by the processor, based at least in part on the lesion likelihood classifications for the hotspots, a subset of the one or more hotspots corresponding to hotspots having a high likelihood of corresponding to cancerous lesions (e.g., for inclusion in a report; e.g., for use in computing one or more risk index values for the subject).

In another aspect, the invention is directed to a method of measuring intensity values within a reference volume corresponding to a reference tissue region (e.g., a liver volume associated with a liver of a subject) so as to avoid impact from tissue regions associated with low (e.g., abnormally low) radiopharmaceutical uptake (e.g., due to tumors without tracer uptake), the method comprising: (a) receiving (e.g., and/or accessing), by a processor of a computing device, the 3D functional image of a subject, said 3D functional image obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) identifying, by the processor, the reference volume within the 3D functional image; (c) fitting, by the processor, a multi-component mixture model (e.g., a two-component Gaussian mixture model) to intensities of voxels within the reference volume [e.g., fitting the multi-component mixture model to a distribution (e.g., a histogram) of intensities of voxels within the reference volume]; (d) identifying, by the processor, a major mode of the multi-component model; (e) determining, by the processor, a measure of (e.g., a mean, a maximum, a mode, a median, etc.) intensities corresponding to the major mode, thereby determining a reference intensity value corresponding to a measure of intensity of voxels that are (i) within the reference tissue volume and (ii) associated with the major mode (e.g., and excluding, from the reference value calculation, voxels having intensities associated with minor modes) (e.g., thereby avoiding impact from tissue regions associated with low radiopharmaceutical uptake); (f) detecting, by the processor, within the functional image, one or more hotspots corresponding potential cancerous lesions; and (g) determining, by the processes or, for each hotspot of at least a portion of the detected hotspots, a lesion index value, using at least the reference intensity value [e.g., the lesion index value based on (i) a measure of intensities of voxels corresponding to the detected hotspot and (ii) the reference intensity value].

In another aspect, the invention is directed to a method of correcting for intensity bleed (e.g., cross-talk) from due to high-uptake tissue regions within the subject that are associated with high radiopharmaceutical uptake under normal circumstances (e.g., and not necessarily indicative of cancer), the method comprising: (a) receiving (e.g., and/or accessing), by a processor of a computing device, the 3D functional image of the subject, said 3D functional image obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) identifying, by the processor, a high-intensity volume within the 3D functional image, said high intensity volume corresponding to a particular high-uptake tissue region (e.g., a kidney; e.g., a liver; e.g., a bladder) in which high radiopharmaceutical uptake occurs under normal circumstances; (c) identifying, by the processor, based on the identified high-intensity volume, a suppression volume within the 3D functional image, said suppression volume corresponding to a volume lying outside and within a predetermined decay distance from a boundary of the identified high intensity volume; (d) determining, by the processor, a background image corresponding to the 3D functional image with intensities of voxels within the high-intensity volume replaced with interpolated values determined based on intensities of voxels of the 3D functional image within the suppression volume; (e) determining, by the processor, an estimation image by subtracting intensities of voxels of the background image from intensities of voxels from the 3D functional image (e.g., performing a voxel-by-voxel subtraction); (f) determining, by the processor, a suppression map by: extrapolating intensities of voxels of the estimation image corresponding to the high-intensity volume to locations of voxels within the suppression volume to determine intensities of voxels of the suppression map corresponding to the suppression volume; and setting intensities of voxels of the suppression map corresponding to locations outside the suppression volume to zero; and (g) adjusting, by the processor, intensities of voxels of the 3D functional image based on the suppression map (e.g., by subtracting intensities of voxels of the suppression map from intensities of voxels of the 3D functional image), thereby correcting for intensity bleed from the high-intensity volume.

In certain embodiments, the method comprises performing steps (b) through (g) for each of a plurality of high-intensity volumes in a sequential manner, thereby correcting for intensity bleed from each of the plurality of high-intensity volumes.

In certain embodiments, the plurality of high-intensity volumes comprise one or more members selected from the group consisting of a kidney, a liver, and a bladder (e.g., a urinary bladder).

In another aspect, the invention is directed to a method for automatically processing 3D images of a subject to identify and/or characterize (e.g., grade) cancerous lesions within the subject, the method comprising: (a) receiving (e.g., and/or accessing), by a processor of a computing device, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) automatically detecting, by the processor, one or more hotspots within the 3D functional image, each hotspot corresponding to a local region of elevated intensity with respect to its surrounding and representing (e.g., indicative of) a potential cancerous lesion within the subject; (c) causing, by the processor, rendering of a graphical representation of the one or more hotspots for display within an interactive graphical user interface (GUI) (e.g., a quality control and reporting GUI); (d) receiving, by the processor, via the interactive GUI, a user selection of a final hotspot set comprising at least a portion (e.g., up to all) of the one or more automatically detected hotspots (e.g., for inclusion in a report); and (e) storing and/or providing for display and/or further processing, the final hotspot set.

In certain embodiments, the method comprises: (f) receiving, by the processor, via the GUI, a user selection of one or more additional, user-identified, hotspots for inclusion in the final hotspot set; and (g) updating, by the processor, the final hotspot set to include the one or more additional user-identified hotspots.

In certain embodiments, step (b) comprises using one or more machine learning modules.

In another aspect, the invention is directed to a method for automatically processing 3D images of a subject to identify and characterize (e.g., grade) cancerous lesions within the subject, the method comprising: (a) receiving (e.g., and/or accessing), by a processor of a computing device, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) automatically detecting, by the processor, one or more hotspots within the 3D functional image, each hotspot corresponding to a local region of elevated intensity with respect to its surrounding and representing (e.g., indicative of) a potential cancerous lesion within the subject; (c) automatically determining, by the processor, for each of at least a portion of the one or more hotspots, an anatomical classification corresponding to a particular anatomical region and/or group of anatomical regions within the subject in which the potential cancerous lesion that the hotspot represents is determined [e.g., by the processor (e.g., based on a received and/or determined 3D segmentation map)] to be located [e.g., within a prostate, a pelvic lymph node, a non-pelvic lymph node, a bone (e.g., a bone metastatic region), and a soft tissue region not situated in prostate or lymph node]; and (d) storing and/or providing for display and/or further processing, an identification of the one or more hotspots along with, for each hotspot, the anatomical classification corresponding to the hotspot.

In certain embodiments, step (b) comprises using one or more machine learning modules.

In another aspect, the invention is directed to a system for automatically processing 3D images of a subject to identify and/or characterize (e.g., grade) cancerous lesions within the subject, the system comprising: a processor of a computing device; and a memory having instructions stored thereon, wherein the instructions, when executed by the processor, cause the processor to: (a) receive (e.g., and/or access) a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value (e.g., standard uptake value (SUV)) that represents detected radiation emitted from the particular physical volume, wherein at least a portion of the plurality of voxels of the 3D functional image represent physical volumes within the target tissue region]; (b) automatically detect, using a machine learning module [e.g., a pre-trained machine learning module (e.g., having pre-determined (e.g., and fixed) parameters having been determined via a training procedure)], one or more hotspots within the 3D functional image, each hotspot corresponding to a local region of elevated intensity with respect to its surrounding and representing (e.g., indicative of) a potential cancerous lesion within the subject, thereby creating one or both of (i) and (ii) as follows: (i) a hotspot list [e.g., a list of coordinates (e.g., image coordinates; e.g., physical space coordinates); e.g., a mask identifying voxels of the 3D functional image, each voxel corresponding to a location (e.g., a center of mass) of a detected hotspot] identifying, for each hotspot, a location of the hotspot, and (ii) a 3D hotspot map, identifying, for each hotspot, a corresponding 3D hotspot volume within the 3D functional image {e.g., wherein, the 3D hotspot map is a segmentation map (e.g., comprising one or more segmentation masks) identifying, for each hotspot, voxels within the 3D functional image corresponding to the 3D hotspot volume of each hotspot [e.g., wherein the 3D hotspot map is obtained via artificial intelligence-based segmentation of the functional image (e.g., using a machine-learning module that receives, as input, at least the 3D functional image and generates the 3D hotspot map as output, thereby segmenting hotspots)]; e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)}; and (c) store and/or provide, for display and/or further processing, the hotspot list and/or the 3D hotspot map.

In certain embodiments, the machine learning module receives, as input, at least a portion of the 3D functional image and automatically detects the one or more hotspots based at least in part on intensities of voxels of the received portion of the 3D functional image.

In certain embodiments, the machine learning module receives, as input, a 3D segmentation map that identifies one or more volumes of interest (VOIs) within the 3D functional image, each VOI corresponding to a particular target tissue region and/or a particular anatomical region within the subject [e.g., a soft-tissue region (e.g., a prostate, a lymph node, a lung, a breast); e.g., one or more particular bones; e.g., an overall skeletal region].

In certain embodiments, the instructions cause the processor to: receive (e.g., and/or access) a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject, and the machine learning module receives at least two channels of input, said input channels comprising a first input channel corresponding to at least a portion of the 3D anatomical image and a second input channel corresponding to at least a portion of the 3D functional image [e.g., wherein the machine learning module receives a PET image and a CT image as separate channels (e.g., separate channels representative of the same volume) (e.g., analogous to receipt by a machine learning module of two color channels (RGB) of a photographic color image)].

In certain embodiments, the machine learning module receives, as input, a 3D segmentation map that identifies, within the 3D functional image and/or the 3D anatomical image, one or more volumes of interest (VOIs), each VOI corresponding to a particular target tissue region and/or a particular anatomical region.

In certain embodiments, the instructions cause the processor to automatically segment the 3D anatomical image, thereby creating the 3D segmentation map.

In certain embodiments, the machine learning module is a region-specific machine learning module that receives, as input, a specific portion of the 3D functional image corresponding to one or more specific tissue regions and/or anatomical regions of the subject.

In certain embodiments, the machine learning module generates, as output, the hotspot list [e.g., wherein the machine learning module implements a machine learning algorithm (e.g., an artificial neural network (ANN)) trained to determine, based on intensities of voxels of at least a portion of the 3D functional image, one or more locations (e.g., 3D coordinates), each corresponding to a location of one of the one or more hotspots].

In certain embodiments, the machine learning module generates, as output, the 3D hotspot map [e.g., wherein the machine learning module implements a machine learning algorithm (e.g., an artificial neural network (ANN)) trained to segment the 3D functional image (e.g., based at least in part on intensities of voxels of the 3D functional image) to identify the 3D hotspot volumes of the 3D hotspot map (e.g., the 3D hotspot map delineating, for each hotspot, a 3D boundary (e.g., an irregular boundary) of the hotspot, thereby identifying the 3D hotspot volumes (e.g., enclosed by the 3D hotspot boundaries)); e.g., wherein the machine learning module implements a machine learning algorithm trained to determine, for each voxel of at least a portion of the 3D functional image, a hotspot likelihood value representing a likelihood that the voxel corresponds to a hotspot (e.g., and step (b) comprises performing one or more subsequent post-processing steps, such as thresholding, to identify the 3D hotspot volumes of the 3D hotspot map using the hotspot likelihood values (e.g., the 3D hotspot map delineating, for each hotspot, a 3D boundary (e.g., an irregular boundary) of the hotspot, thereby identifying the 3D hotspot volumes (e.g., enclosed by the 3D hotspot boundaries)))].

In certain embodiments, the instructions cause the processor to: (d) determine, for each hotspot of at least a portion of the hotspots, a lesion likelihood classification corresponding to a likelihood of the hotspot representing a lesion within the subject [e.g., a binary classification indicative of whether the hotspot is a true lesion or not; e.g., a likelihood value on a scale (e.g., a floating point value ranging from zero to one) representing a likelihood of the hotspot representing a true lesion].

In certain embodiments, at step (d) the instructions cause the processor to use the machine learning module to determine, for each hotspot of the portion, the lesion likelihood classification [e.g., wherein the machine learning module implements a machine learning algorithm trained to detect hotspots (e.g., to generate, as output, the hotspot list and/or the 3D hotspot map) and to determine, for each hotspot, the lesion likelihood classification for the hotpot].

In certain embodiments, at step (d) the instructions cause the processor to use a second machine learning module (e.g., a hotspot classification module) to determine the lesion likelihood classification for each hotspot [e.g., based at least in part on one or more members selected from the group consisting of intensities of the 3D functional image, the hotspot list, the 3D hotspot map, intensities of a 3D anatomical image, and a 3D segmentation map; e.g., wherein the second machine learning module receives one or more channels of input corresponding to one or more members selected from the group consisting of: intensities of the 3D functional image, the hotspot list, the 3D hotspot map, intensities of a 3D anatomical image, and a 3D segmentation map].

In certain embodiments, the instructions cause the processor to determine, for each hotspot, a set of one or more hotspot features and using the set of the one or more hotspot features as input to the second machine learning module.

In certain embodiments, 55 to 58, wherein the instructions cause the processor to: (e) select, based at least in part on the lesion likelihood classifications for the hotspots, a subset of the one or more hotspots corresponding to hotspots having a high likelihood of corresponding to cancerous lesions (e.g., for inclusion in a report; e.g., for use in computing one or more risk index values for the subject).

In certain embodiments, the instructions cause the processor to: (f) [e.g., prior to step (b)] adjust intensities of voxels of the 3D functional image, by the processor, to correct for intensity bleed (e.g., cross-talk) from one or more high-intensity volumes of the 3D functional image, each of the one or more high-intensity volumes corresponding to a high-uptake tissue region within the subject associated with high radiopharmaceutical uptake under normal circumstances (e.g., not necessarily indicative of cancer).

In certain embodiments, at step (f) the instructions cause the processor to correct for intensity bleed from a plurality of high-intensity volumes one at a time, in a sequential fashion [e.g., first adjusting intensities of voxels of the 3D functional image to correct for intensity bleed from a first high-intensity volume to generate a first corrected image, then adjusting intensities of voxels of the first corrected image to correct for intensity bleed from a second high-intensity volume, and so on].

In certain embodiments, the one or more high-intensity volumes correspond to one or more high-uptake tissue regions selected from the group consisting of a kidney, a liver, and a bladder (e.g., a urinary bladder).

In certain embodiments, the instructions cause the processor to: (g) determine, for each of at least a portion of the one or more hotspots, a corresponding lesion index indicative of a level of radiopharmaceutical uptake within and/or size (e.g., volume) of an underlying lesion to which the hotspot corresponds.

In certain embodiments, at step (g) the instructions cause the processor to compare an intensity (intensities) (e.g., corresponding to standard uptake values (SUVs)) of one or more voxels associated with the hotspot (e.g., at and/or about a location of the hotspot; e.g., within a volume of the hotspot) with one or more reference values, each reference value associated with a particular reference tissue region (e.g., a liver; e.g., an aorta portion) within the subject and determined based on intensities (e.g., SUV values) of a reference volume corresponding to the reference tissue region [e.g., as an average (e.g., a robust average, such as a mean of values in an interquartile range)].

In certain embodiments, the one or more reference values comprise one or more members selected from the group consisting of an aorta reference value associated with an aorta portion of the subject and a liver reference value associated with a liver of the subject.

In certain embodiments, for at least one particular reference value associated with a particular reference tissue region, the instructions cause the processor to determine the particular reference value by fitting intensities of voxels [e.g., by fitting an distribution of intensities of voxels (e.g., fitting a histogram of voxel intensities)] within a particular reference volume corresponding to the particular reference tissue region to a multi-component mixture model (e.g., a two-component Gaussian model) [e.g., and identifying one or more minor peaks in a distribution of voxel intensities, said minor peaks corresponding to voxels associated with anomalous uptake, and excluding, from the reference value determination, those voxels from the reference value determination (e.g., thereby accounting for effects of abnormally low radiopharmaceutical uptake in certain portions of reference tissue regions, such as portions of the liver)].

In certain embodiments, the instructions cause the processor to use the determined lesion index values compute (e.g., automatically) an overall risk index for the subject, indicative of a caner status and/or risk for the subject.

In certain embodiments, the instructions cause the processor to determine (e.g., automatically), for each hotspot, an anatomical classification corresponding to a particular anatomical region and/or group of anatomical regions within the subject in which the potential cancerous lesion that the hotspot represents is determined [e.g., by the processor (e.g., based on a received and/or determined 3D segmentation map)] to be located [e.g., within a prostate, a pelvic lymph node, a non-pelvic lymph node, a bone (e.g., a bone metastatic region), and a soft tissue region not situated in prostate or lymph node].

In certain embodiments, the instructions cause the processor to: (h) causing, for display within a graphical user interface (GUI), rendering of a graphical representation of at least a portion of the one or more hotspots for review by a user.

In certain embodiments, the instructions cause the processor to: (i) receiving, via the GUI, a user selection of a subset of the one or more hotspots confirmed via user review as likely to represent underlying cancerous lesions within the subject.

In certain embodiments, the 3D functional image comprises a PET or SPECT image obtained following administration of an agent (e.g., a radiopharmaceutical; e.g., an imaging agent) to the subject. In certain embodiments, the agent comprises a PSMA binding agent. In certain embodiments, the agent comprises ¹⁸F. In certain embodiments, the agent comprises [18F]DCFPyL. In certain embodiments, the agent comprises the agent comprises PSMA-11 (e.g., ⁶⁸Ga-PSMA-11). In certain embodiments, the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

In certain embodiments, the machine learning module implements a neural network [e.g., an artificial neural network (ANN); e.g., a convolutional neural network (CNN)].

In certain embodiments, the processor is a processor of a cloud-based system.

In another aspect, the invention is directed to a system for automatically processing 3D images of a subject to identify and/or characterize (e.g., grade) cancerous lesions within the subject, the system comprising: a processor of a computing device; and a memory having instructions stored thereon, wherein the instructions, when executed by the processor, cause the processor to: (a) receive (e.g., and/or access) a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) receive (e.g., and/or access) a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject; (c) automatically detect, using a machine learning module, one or more hotspots within the 3D functional image, each hotspot corresponding to a local region of elevated intensity with respect to its surrounding and representing (e.g., indicative of) a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image {e.g., wherein, the 3D hotspot map is a segmentation map (e.g., comprising one or more segmentation masks) identifying, for each hotspot, voxels within the 3D functional image corresponding to the 3D hotspot volume of each hotspot [e.g., wherein the 3D hotspot map is obtained via artificial intelligence-based segmentation of the functional image (e.g., using a machine-learning module that receives, as input, at least the 3D functional image and generates the 3D hotspot map as output, thereby segmenting hotspots)]; e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)}, wherein the machine learning module receives at least two channels of input, said input channels comprising a first input channel corresponding to at least a portion of the 3D anatomical image and a second input channel corresponding to at least a portion of the 3D functional image [e.g., wherein the machine learning module receives a PET image and a CT image as separate channels (e.g., separate channels representative of the same volume) (e.g., analogous to receipt by a machine learning module of two color channels (RGB) of a photographic color image)] and/or anatomical information derived therefrom [e.g., a 3D segmentation map that identifies, within the 3D functional image, one or more volumes of interest (VOIs), each VOI corresponding to a particular target tissue region and/or a particular anatomical region]; and (d) store and/or provide, for display and/or further processing, the hotspot list and/or the 3D hotspot map.

In another aspect, the invention is directed to a system for automatically processing 3D images of a subject to identify and/or characterize (e.g., grade) cancerous lesions within the subject, the system comprising: a processor of a computing device; and a memory having instructions stored thereon, wherein the instructions, when executed by the processor, cause the processor to: (a) receive (e.g., and/or access) a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) automatically detect, using a first machine learning module, one or more hotspots within the 3D functional image, each hotspot corresponding to a local region of elevated intensity with respect to its surrounding and representing (e.g., indicative of) a potential cancerous lesion within the subject, thereby creating a hotspot list identifying, for each hotspot, a location of the hotspot [e.g., wherein the machine learning module implements a machine learning algorithm (e.g., an artificial neural network (ANN)) trained to determine, based on intensities of voxels of at least a portion of the 3D functional image, one or more locations (e.g., 3D coordinates), each corresponding to a location of one of the one or more hotspots]; (c) automatically determine, using a second machine learning module and the hotspot list, for each of the one or more hotspots, a corresponding 3D hotspot volume within the 3D functional image, thereby creating a 3D hotspot map [e.g., wherein the second machine learning module implements a machine learning algorithm (e.g., an artificial neural network (ANN)) trained to segment the 3D functional image based at least in part on the hotspot list along with intensities of voxels of the 3D functional image to identify the 3D hotspot volumes of the 3D hotspot map; e.g., wherein the machine learning module implements a machine learning algorithm trained to determine, for each voxel of at least a portion of the 3D functional image, a hotspot likelihood value representing a likelihood that the voxel corresponds to a hotspot (e.g., and step (b) comprises performing one or more subsequent post-processing steps, such as thresholding, to identify the 3D hotspot volumes of the 3D hotspot map using the hotspot likelihood values][e.g., wherein, the 3D hotspot map is a segmentation map (e.g., comprising one or more segmentation masks) generated using (e.g., based on and/or corresponding to output from) the second machine learning module, the 3D hotspot map identifying, for each hotspot, voxels within the 3D functional image corresponding to the 3D hotspot volume of each hotspot); e.g., wherein the 3D hotspot map delineates, for each hotspot, a 3D boundary (e.g., an irregular boundary) of the hotspot (e.g., the 3D boundary enclosing the 3D hotspot volume, e.g., and distinguishing voxels of the 3D functional image that make up the 3D hotspot volume from other voxels of the 3D functional image)]; and (d) store and/or provide, for display and/or further processing, the hotspot list and/or the 3D hotspot map.

In certain embodiments, the instructions cause the processor to: (e) determine, for each hotspot of at least a portion of the hotspots, a lesion likelihood classification corresponding to a likelihood of the hotspot representing a lesion within the subject.

In certain embodiments, at step (e) the instructions cause the processor to use a third machine learning module (e.g., a hotspot classification module) to determine the lesion likelihood classification for each hotspot [e.g., based at least in part on one or more members selected from the group consisting of intensities of the 3D functional image, the hotspot list, the 3D hotspot map, intensities of a 3D anatomical image, and a 3D segmentation map; e.g., wherein the third machine learning module receives one or more channels of input corresponding to one or more members selected from the group consisting of intensities of the 3D functional image, the hotspot list, the 3D hotspot map, intensities of a 3D anatomical image, and a 3D segmentation map].

In certain embodiments, the instructions cause the processor to: (f) select, based at least in part on the lesion likelihood classifications for the hotspots, a subset of the one or more hotspots corresponding to hotspots having a high likelihood of corresponding to cancerous lesions (e.g., for inclusion in a report; e.g., for use in computing one or more risk index values for the subject).

In another aspect, the invention is directed to a system for measuring intensity values within a reference volume corresponding to a reference tissue region (e.g., a liver volume associated with a liver of a subject) so as to avoid impact from tissue regions associated with low (e.g., abnormally low) radiopharmaceutical uptake (e.g., due to tumors without tracer uptake), the system comprising: a processor of a computing device; and a memory having instructions stored thereon, wherein the instructions, when executed by the processor, cause the processor to: (a) receive (e.g., and/or access) a 3D functional image of a subject, said 3D functional image obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) identify the reference volume within the 3D functional image; (c) fit a multi-component mixture model (e.g., a two-component Gaussian mixture model) to intensities of voxels within the reference volume [e.g., fitting the multi-component mixture model to a distribution (e.g., a histogram) of intensities of voxels within the reference volume]; (d) identify a major mode of the multi-component model; (e) determine a measure of (e.g., a mean, a maximum, a mode, a median, etc.) intensities corresponding to the major mode, thereby determining a reference intensity value corresponding to a measure of intensity of voxels that are (i) within the reference tissue volume and (ii) associated with the major mode, (e.g., and excluding, from the reference value calculation, voxels having intensities associated with minor modes) (e.g., thereby avoiding impact from tissue regions associated with low radiopharmaceutical uptake); (f) detect, within the 3D functional image, one or more hotspots corresponding potential cancerous lesions; and (g) determine, for each hotspot of at least a portion of the detected hotspots, a lesion index value, using at least the reference intensity value [e.g., the lesion index value based on (i) a measure of intensities of voxels corresponding to the detected hotspot and (ii) the reference intensity value]. In another aspect, the invention is directed to a system for correcting for intensity bleed (e.g., cross-talk) from due to high-uptake tissue regions within the subject that are associated with high radiopharmaceutical uptake under normal circumstances (e.g., and not necessarily indicative of cancer), the method comprising: (a) receive (e.g., and/or access) a 3D functional image of the subject, said 3D functional image obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) identify a high-intensity volume within the 3D functional image, said high intensity volume corresponding to a particular high-uptake tissue region (e.g., a kidney; e.g., a liver; e.g., a bladder) in which high radiopharmaceutical uptake occurs under normal circumstances; (c) identify, based on the identified high-intensity volume, a suppression volume within the 3D functional image, said suppression volume corresponding to a volume lying outside and within a predetermined decay distance from a boundary of the identified high intensity volume; (d) determine a background image corresponding to the 3D functional image with intensities of voxels within the high-intensity volume replaced with interpolated values determined based on intensities of voxels of the 3D functional image within the suppression volume; (e) determine an estimation image by subtracting intensities of voxels of the background image from intensities of voxels from the 3D functional image (e.g., performing a voxel-by-voxel subtraction); (f) determine a suppression map by: extrapolating intensities of voxels of the estimation image corresponding to the high-intensity volume to locations of voxels within the suppression volume to determine intensities of voxels of the suppression map corresponding to the suppression volume; and setting intensities of voxels of the suppression map corresponding to locations outside the suppression volume to zero; and (g) adjust intensities of voxels of the 3D functional image based on the suppression map (e.g., by subtracting intensities of voxels of the suppression map from intensities of voxels of the 3D functional image), thereby correcting for intensity bleed from the high-intensity volume.

In certain embodiments, the instructions cause the processor to perform steps (b) through (g) for each of a plurality of high-intensity volumes in a sequential manner, thereby correcting for intensity bleed from each of the plurality of high-intensity volumes.

In certain embodiments, the plurality of high-intensity volumes comprise one or more members selected from the group consisting of a kidney, a liver, and a bladder (e.g., a urinary bladder).

In another aspect, the invention is directed to a system for automatically processing 3D images of a subject to identify and/or characterize (e.g., grade) cancerous lesions within the subject, the system comprising: a processor of a computing device; and a memory having instructions stored thereon, wherein the instructions, when executed by the processor, cause the processor to: (a) receive (e.g., and/or access), a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (b) automatically detect one or more hotspots within the 3D functional image, each hotspot corresponding to a local region of elevated intensity with respect to its surrounding and representing (e.g., indicative of) a potential cancerous lesion within the subject; (c) cause rendering of a graphical representation of the one or more hotspots for display within an interactive graphical user interface (GUI) (e.g., a quality control and reporting GUI); (d) receive, via the interactive GUI, a user selection of a final hotspot set comprising at least a portion (e.g., up to all) of the one or more automatically detected hotspots (e.g., for inclusion in a report); and (e) store and/or provide, for display and/or further processing, the final hotspot set.

In certain embodiments, the instructions cause the processor to: (f) receive, via the GUI, a user selection of one or more additional, user-identified, hotspots for inclusion in the final hotspot set; and (g) update, the final hotspot set to include the one or more additional user-identified hotspots.

In certain embodiments, at step (b) the instructions cause the processor to use one or more machine learning modules.

In another aspect, the invention is directed to a method for automatically processing 3D images of a subject to identify and/or characterize (e.g., grade) cancerous lesions within the subject, the method comprising: (a) receiving (e.g., and/or accessing), by a processor of a computing device, a 3D functional image [e.g., positron emission tomography (PET); e.g., single-photon emission computed tomography (SPECT)] of the subject obtained using a functional imaging modality; (b) receiving (e.g., and/or accessing), by the processor, a 3D anatomical image [e.g., a computed tomography (CT) image; e.g., a magnetic resonance (MR) image] of the subject obtained using an anatomical imaging modality; (c) receiving (e.g., and or accessing), by the processor, a 3D segmentation map identifying one or more particular tissue region(s) or group(s) of tissue regions (e.g., a set of tissue regions corresponding to a particular anatomical region; e.g., a group of tissue regions comprising organs in which high or low radiopharmaceutical uptake occurs) within the 3D functional image and/or within the 3D anatomical image; (d) automatically detecting and/or segmenting, by the processor, using one or more machine learning module(s), a set of one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot [e.g., as detected by the one or more machine learning module(s)], and (ii) a 3D hotspot map, identifying, for each hotspot, a corresponding 3D hotspot volume within the 3D functional image [e.g., as determined via segmentation performed by the one or more machine learning module(s)] [e.g., wherein the 3D hotspot map is a segmentation map that delineates, for each hotspot, a 3D hotspot boundary (e.g., an irregular boundary) of the hotspot (e.g., the 3D boundary enclosing the 3D hotspot volume)], wherein at least one (e.g., up to all) of the one or more machine learning module(s) receives, as input (i) the 3D functional image, (ii) the 3D anatomical image, and (iii) the 3D segmentation map; and (e) storing and/or providing, for display and/or further processing, a hotspot list and/or the 3D hotspot map.

In certain embodiments, the method comprises: receiving, by the processor, an initial 3D segmentation map that identifies one or more (e.g., a plurality) particular tissue regions (e.g., organs and/or particular bones) within the 3D anatomical image and/or the 3D functional image; and identifying, by the processor, at least a portion of the one or more particular tissue regions as belonging to a particular one of one or more tissue grouping(s) (e.g., pre-defined groupings) and updating, by the processor, the 3D segmentation map to indicate the identified particular regions as belonging to the particular tissue grouping; and using, by the processor, the updated 3D segmentation map as input to at least one of the one or more machine learning modules.

In certain embodiments, the one or more tissue groupings comprise a soft-tissue grouping, such that particular tissue regions that represent soft-tissue are identified as belonging to the soft-tissue grouping. In certain embodiments, the one or more tissue groupings comprise a bone tissue grouping, such that particular tissue regions that represent bone are identified as belonging to the bone tissue grouping. In certain embodiments, the one or more tissue groupings comprise a high-uptake organ grouping, such that one or more organs associated with high radiopharmaceutical uptake (e.g., under normal circumstances, and not necessarily due to presence of lesions) are identified as belonging to the high uptake grouping.

In certain embodiments, the method comprises, for each detected and/or segmented hotspot, determining, by the processor, a classification for the hotspot [e.g., according to anatomical location, e.g., classifying the hotspot as bone, lymph, or prostate, e.g., assigning an alphanumeric code based on a determined (e.g., by the processor) location of the hotspot in the subject, such as the labeling scheme in Table 1].

In certain embodiments, the method comprises using at least one of the one or more machine learning modules to determine, for each detected and/or segmented lesion, the classification for the hotspot (e.g., wherein a single machine learning module performs detection, segmentation, and classification).

In certain embodiments, the one or more machine learning modules comprise: (A) a full body lesion detection module that detects and/or segments hotspots throughout an entire body; and (B) a prostate lesion module that detects and/or segments hotspots within the prostate. In certain embodiments, the method comprises generating hotspot list and/or maps using each of (A) and (B) and merging the results.

In certain embodiments, step (d) comprises: segmenting and classifying the set of one or more hotspots to create a labeled 3D hotspot map that identifies, for each hotspot, a corresponding 3D hotspot volume within the 3D functional image and in which each hotspot volume is labeled as belonging to a particular hotspot class of a plurality of hotspot classes [e.g., each hotspot class identifying a particular anatomical and/or tissue region (e.g., lymph, bone, prostate) that a lesion represented by the hotspot is determined to be located] by: using a first machine learning module to segment a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class [e.g., identifying all hotspots as belonging to a single hotspot class, so as to differentiate between background regions and hotspot volumes (e.g., but not between different types of hotspots) (e.g., such that each hotspot volume identified by the first 3D hotspot map is labeled as belonging to a single hotspot class, as opposed to background)]; using a second machine learning module to segment a second initial set of one or more hotspots within the 3D functional image, thereby creating a second initial 3D hotspot map that identifies a second set of initial hotspot volumes, wherein the second machine learning module segments the 3D functional image to according to the plurality of different hotspot classes, such that the second initial 3D hotspot map is a multi-class 3D hotspot map in which each hotspot volume is labeled as belonging to a particular one of the plurality of different hotspot classes (e.g., so as to differentiate between hotspot volumes corresponding to different hotspot classes, as well between hotspot volumes and as background regions); and merging, by the processor, the first initial 3D hotspot map and the second initial 3D hotspot map by, for at least a portion of the hotspot volumes identified by the first initial 3D hotspot map: identifying a matching hotspot volume of the second initial 3D hotspot map (e.g., by identifying substantially overlapping hotspot volumes of the first and second initial 3D hotspot maps), the matching hotspot volume of the second 3D hotspot map having been labeled as belonging to a particular hotspot class of the plurality of different hotspot classes; and labeling the particular hotspot volume of the first initial 3D hotspot map as belonging to the particular hotspot class (e.g., that the matching hotspot volume is labeled as belonging to), thereby creating a merged 3D hotspot map that includes segmented hotspot volumes of the first 3D hotspot map having been labeled according classes that matching hotspot volumes of the second 3D hotspot map are identified as belonging to; and step (e) comprises storing and/or providing, for display and/or further processing, the merged 3D hotspot map.

In certain embodiments, the plurality of different hotspot classes comprise one or more members selected from the group consisting of: (i) bone hotspots, determined (e.g., by the second machine learning module) to represent lesions located in bone, (ii) lymph hotspots, determined (e.g., by the second machine learning module) to represent lesions located in lymph nodes, and (iii) prostate hotspots, determined (e.g., by the second machine learning module) to represent lesions located in a prostate.

In certain embodiments, the method further comprises: (f) receiving and/or accessing the hotspot list; and (g) for each hotspot in the hotspot list, segmenting the hotspot using an analytical model [e.g., thereby creating a 3D map of analytically segmented hotspots (e.g., the 3D map identifying, for each hotspot, a hotspot volume comprising voxels of the 3D anatomical image and/or functional image enclosed by the segmented hotspot region)].

In certain embodiments, the method further comprises: (h) receiving and/or accessing the hotspot map; and (i) for each hotspot in the hotspot map, segmenting the hotspot using an analytical model [e.g., thereby creating a 3D map of analytically segmented hotspots (e.g., the 3D map identifying, for each hotspot, a hotspot volume comprising voxels of the 3D anatomical image and/or functional image enclosed by the segmented hotspot region)].

In certain embodiments, the analytical model is an adaptive thresholding method, and step (i) comprises: determining one or more reference values, each based on a measure of intensities of voxels of the 3D functional image located within a particular reference volume corresponding to a particular reference tissue region (e.g., a blood pool reference value determined based on intensities within an aorta volume corresponding to a portion of an aorta of a subject; e.g., a liver reference value determined based on intensities within a liver volume corresponding to a liver of a subject); and for each particular hotspot volume of the 3D hotspot map: determining, by the processor, a corresponding hotspot intensity based on intensities of voxels within the particular hotspot volume [e.g., wherein the hotspot intensity is a maximum of intensities (e.g., representing SUVs) of voxels within the particular hotspot volume]; and determining, by the processor, a hotspot-specific threshold value for the particular hotspot based on (i) the corresponding hotspot intensity and (ii) at least one of the one or more reference value(s).

In certain embodiments, the hotspot-specific threshold value is determined using a particular threshold function selected from a plurality of threshold functions, the particular threshold function selected based a comparison of the corresponding hotspot intensity with the at least one reference value [e.g., wherein each of the plurality of threshold functions is associated with a particular range of intensity (e.g., SUV) values, and the particular threshold function is selected according to the particular range that the hotspot intensity and/or a (e.g., predetermined) percentage thereof falls within (e.g., and wherein each particular range of intensity values is bounded at least in part by a multiple of the at least one reference value)].

In certain embodiments, the hotspot-specific threshold value is determined (e.g., by the particular threshold function) as a variable percentage of the corresponding hotspot intensity, wherein the variable percentage decreases with increasing hotspot intensity [e.g., wherein the variable percentage is itself a function (e.g., a decreasing function) of the corresponding hotspot intensity].

In another aspect, the invention is directed to a method for automatically processing 3D images of a subject to identify and/or characterize (e.g., grade, e.g., classify, e.g., as representing a particular lesion type) cancerous lesions within the subject, the method comprising: (a) receiving (e.g., and/or accessing), by a processor of a computing device, a 3D functional image [e.g., positron emission tomography (PET); e.g., single-photon emission computed tomography (SPECT)] of the subject obtained using a functional imaging modality; (b) automatically segmenting, by the processor, using a first machine learning module, a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class [e.g., identifying all hotspots as belonging to a single hotspot class, so as to differentiate between background regions and hotspot volumes (e.g., but not between different types of hotspots) (e.g., such that each hotspot volume identified by the first 3D hotspot map is labeled as belonging to a single hotspot class, as opposed to background)]; (c) automatically segmenting, by the processor, using a second machine learning module, a second initial set of one or more hotspots within the 3D functional image, thereby creating a second initial 3D hotspot map that identifies a second set of initial hotspot volumes, wherein the second machine learning module segments the 3D functional image to according to a plurality of different hotspot classes [e.g., each hotspot class identifying a particular anatomical and/or tissue region (e.g., lymph, bone, prostate) that a lesion represented by the hotspot is determined to be located], such that the second initial 3D hotspot map is a multi-class 3D hotspot map in which each hotspot volume is labeled as belonging to a particular one of the plurality of different hotspot classes (e.g., so as to differentiate between hotspot volumes corresponding to different hotspot classes, as well between hotspot volumes and as background regions); (d) merging, by the processor, the first initial 3D hotspot map and the second initial 3D hotspot map by, for each particular hotspot volume of at least a portion of the first set of initial hotspot volumes identified by the first initial 3D hotspot map: identifying a matching hotspot volume of the second initial 3D hotspot map (e.g., by identifying substantially overlapping hotspot volumes of the first and second initial 3D hotspot maps), the matching hotspot volume of the second 3D hotspot map having been labeled as belonging to a particular hotspot class of the plurality of different hotspot classes; and labeling the particular hotspot volume of the first initial 3D hotspot map as belonging to the particular hotspot class (that the matching hotspot volume is labeled as belonging to), thereby creating a merged 3D hotspot map that includes segmented hotspot volumes of the first 3D hotspot map having been labeled according classes that matching hotspots of the second 3D hotspot map are identified as belonging to; and (e) storing and/or providing, for display and/or further processing, the merged 3D hotspot map.

In certain embodiments, the plurality of different hotspot classes comprises one or more members selected from the group consisting of: (i) bone hotspots, determined (e.g., by the second machine learning module) to represent lesions located in bone, (ii) lymph hotspots, determined (e.g., by the second machine learning module) to represent lesions located in lymph nodes, and (iii) prostate hotspots, determined (e.g., by the second machine learning module) to represent lesions located in a prostate.

In another aspect, the invention is directed to a method for automatically processing 3D images of a subject to identify and/or characterize (e.g., grade; e.g., classify, e.g., as representing a particular lesion type) cancerous lesions within the subject, via an adaptive thresholding approach the method comprising: (a) receiving (e.g., and/or accessing), by a processor of a computing device, a 3D functional image [e.g., positron emission tomography (PET); e.g., single-photon emission computed tomography (SPECT)] of the subject obtained using a functional imaging modality; (b) receiving (e.g., and/or accessing), by the processor, a preliminary 3D hotspot map identifying, within the 3D functional image, one or more preliminary hotspot volumes; (c) determining, by the processor, one or more reference values, each based on a measure of intensities of voxels of the 3D functional image located within a particular reference volume corresponding to a particular reference tissue region (e.g., a blood pool reference value determined based on intensities within an aorta volume corresponding to a portion of an aorta of a subject; e.g., a liver reference value determined based on intensities within a liver volume corresponding to a liver of a subject); (d) creating, by the processor, a refined 3D hotspot map based on the preliminary hotspot volumes and using an adaptive threshold-based segmentation by, for each particular preliminary hotspot volume of at least a portion of the one or more preliminary hotspot volumes identified by the preliminary 3D hotspot map: determining a corresponding hotspot intensity based on intensities of voxels within the particular preliminary hotspot volume [e.g., wherein the hotspot intensity is a maximum of intensities (e.g., representing SUVs) of voxels within the particular preliminary hotspot volume]; determining a hotspot-specific threshold value for the particular preliminary hotspot volume based on (i) the corresponding hotspot intensity and (ii) at least one of the one or more reference value(s); segmenting at least a portion of the 3D functional (e.g., a sub-volume about in the particular preliminary hotspot volume) using a threshold-based segmentation algorithm that performs image segmentation using the hotspot-specific threshold value determined for the particular preliminary hotspot volume [e.g., and identifies clusters of voxels (e.g., 3D clusters of voxels connected to each other in an n-connected component fashion (e.g., where n = 6, n = 18, etc.)) having intensities above the hotspot-specific threshold value and comprising a maximum intensity voxel of the preliminary hotspot], thereby determining a refined, analytically segmented, hotspot volume corresponding to the particular preliminary hotspot volume; and including the refined hotspot volume in the refined 3D hotspot map; and (e) storing and/or providing, for display and/or further processing, the refined 3D hotspot map.

In certain embodiments, the hotspot-specific threshold value is determined using a particular threshold function selected from a plurality of threshold functions, the particular threshold function selected based a comparison of the corresponding hotspot intensity with the at least one reference value [e.g., wherein each of the plurality of threshold functions is associated with a particular range of intensity (e.g., SUV) values, and the particular threshold function is selected according to the particular range that the hotspot intensity and/or a (e.g., predetermined) percentage thereof falls within (e.g., and wherein each particular range of intensity values is bounded at least in part by a multiple of the at least one reference value)].

In certain embodiments, the hotspot-specific threshold value is determined (e.g., by the particular threshold function) as a variable percentage of the corresponding hotspot intensity, wherein the variable percentage decreases with increasing hotspot intensity [e.g., wherein the variable percentage is itself a function (e.g., a decreasing function) of the corresponding hotspot intensity].

In another aspect, the invention is directed to a method for automatically processing 3D images of a subject to identify and/or characterize (e.g., grade; e.g., classify, e.g., as representing a particular lesion type)) cancerous lesions within the subject, the method comprising: (a) receiving (e.g., and/or accessing), by a processor of a computing device, a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject; (b) automatically segmenting, by the processor, the 3D anatomical image to create a 3D segmentation map that identifies a plurality of volumes of interest (VOIs) in the 3D anatomical image, including a liver volume corresponding to a liver of the subject and an aorta volume corresponding to an aorta portion (e.g., thoracic and/or abdominal portion); (c) receiving (e.g., and/or accessing), by the processor, a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (d) automatically segmenting, by the processor, one or more hotspots within the 3D functional image, each segmented hotspot corresponding to a local region of elevated intensity with respect to its surrounding and representing (e.g., indicative of) a potential cancerous lesion within the subject, thereby identifying one or more automatically segmented hotspot volumes; (e) causing, by the processor, rendering of a graphical representation of the one or more automatically segmented hotspot volumes for display within an interactive graphical user interface (GUI) (e.g., a quality control and reporting GUI); (f) receiving, by the processor, via the interactive GUI, a user selection of a final hotspot set comprising at least a portion (e.g., up to all) of the one or more automatically segmented hotspot volumes; (g) determining, by the processor, for each hotspot volume of the final set, a lesion index value based on (i) intensities of voxels of the functional image corresponding to (e.g., located within) the hotspot volume and (ii) one or more reference values determined using intensities of voxels of the functional image corresponding to the liver volume and the aorta volume; and (e) storing and/or providing for display and/or further processing, the final hotspot set and/or lesion index values.

In certain embodiments, step (b) comprises segmenting the anatomical image such that the 3D segmentation map identifies one or more bone volumes corresponding to one or more bones of the subject, and step (d) comprises identifying, within the functional image, a skeletal volume using the one or more bone volumes and segmenting one or more bone hotspot volumes located within the skeletal volume (e.g., by applying one or more difference of Gaussian filters and thresholding the skeletal volume).

In certain embodiments, step (b) comprises segmenting the anatomical image such that the 3D segmentation map identifies one or more organ volumes corresponding to soft-tissue organs of the subject [e.g., left/right lungs, left/right gluteus maximus, urinary bladder, liver, left/right kidney, gallbladder, spleen, thoracic and abdominal aorta and, optionally (e.g., for patients not having undergone radical prostatectomy, a prostate], and step (d) comprises identifying, within the functional image, one or more soft tissue (e.g., a lymph and, optionally, prostate) volumes using the one or more segmented organ volumes and segmenting one or more lymph and/or prostate hotspot volumes located within the soft tissue volume (e.g., by applying one or more Laplacian of Gaussian filters and thresholding the soft-tissue volume).

In certain embodiments, step (d) further comprises, prior to segmenting the one or more lymph and/or prostate hotspot volumes, adjusting intensities of the functional image to suppress intensity from one or more high-uptake tissue regions (e.g., using one or more suppression methods described herein).

In certain embodiments, step (g) comprises determining a liver reference value using intensities of voxels of the functional image corresponding to the liver volume.

In certain embodiments, the method comprises fitting a two component Gaussian mixture model two a histogram of intensities of functional image voxels corresponding to the liver volume, using the two-component Gaussian mixture model fit to identify and exclude voxels having intensities associated with regions of abnormally low uptake from the liver volume, and determining the liver reference value using intensities of remaining (e.g., not excluded) voxels.

In another aspect, the invention is directed to a system for automatically processing 3D images of a subject to identify and/or characterize (e.g., grade; e.g., classify, e.g., as representing a particular lesion type) cancerous lesions within the subject, the system comprising: a processor of a computing device; and a memory having instruction stored thereon, wherein the instructions, when executed by the processor, cause the processor to: (a) receive (e.g., and/or access) a 3D functional image [e.g., positron emission tomography (PET); e.g., single-photon emission computed tomography (SPECT)] of the subject obtained using a functional imaging modality; (b) receive (e.g., and/or access) a 3D anatomical image [e.g., a computed tomography (CT) image; e.g., a magnetic resonance (MR) image] of the subject obtained using an anatomical imaging modality; (c) receive (e.g., and or access) a 3D segmentation map identifying one or more particular tissue region(s) or group(s) of tissue regions (e.g., a set of tissue regions corresponding to a particular anatomical region; e.g., a group of tissue regions comprising organs in which high or low radiopharmaceutical uptake occurs) within the 3D functional image and/or within the 3D anatomical image; (d) automatically detect and/or segment, using one or more machine learning module(s), a set of one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot [e.g., as detected by the one or more machine learning module(s)], and (ii) a 3D hotspot map, identifying, for each hotspot, a corresponding 3D hotspot volume within the 3D functional image [e.g., as determined via segmentation performed by the one or more machine learning module(s)] [e.g., wherein the 3D hotspot map is a segmentation map that delineates, for each hotspot, a 3D hotspot boundary (e.g., an irregular boundary) of the hotspot (e.g., the 3D boundary enclosing the 3D hotspot volume)], wherein at least one (e.g., up to all) of the one or more machine learning module(s) receives, as input (i) the 3D functional image, (ii) the 3D anatomical image, and (iii) the 3D segmentation map; and (e) store and/or provide, for display and/or further processing, the hotspot list and/or the 3D hotspot map.

In certain embodiments, the instructions cause the processor to: receive an initial 3D segmentation map that identifies one or more (e.g., a plurality) particular tissue regions (e.g., organs and/or particular bones) within the 3D anatomical image and/or the 3D functional image; identify at least a portion of the one or more particular tissue regions as belonging to a particular one of one or more tissue groupings (e.g., pre-defined groupings) and update the 3D segmentation map to indicate the identified particular regions as belonging to the particular tissue grouping; and use the updated 3D segmentation map as input to at least one of the one or more machine learning modules.

In certain embodiments, the one or more tissue groupings comprise a soft-tissue grouping, such that particular tissue regions that represent soft-tissue are identified as belonging to the soft-tissue grouping. In certain embodiments, the one or more tissue groupings comprise a bone tissue grouping, such that particular tissue regions that represent bone are identified as belonging to the bone tissue grouping. In certain embodiments, the one or more tissue groupings comprise a high-uptake organ grouping, such that one or more organs associated with high radiopharmaceutical uptake (e.g., under normal circumstances, and not necessarily due to presence of lesions) are identified as belonging to the high uptake grouping.

In certain embodiments, the instructions cause the processor to, for each detected and/or segmented hotspot, determine a classification for the hotspot [e.g., according to anatomical location, e.g., classifying the lesion as bone, lymph, or prostate, e.g., assigning an alphanumeric code based on a determined (e.g., by the processor) location of the hotspot with respect to the subject, such as the labeling scheme in Table 1].

In certain embodiments, the instructions cause the processor to use at least one of the one or more machine learning modules to determine, for each detected and/or segmented hotspot, the classification for the hotspot (e.g., wherein a single machine learning module performs detection, segmentation, and classification).

In certain embodiments, the one or more machine learning modules comprise: (A) a full body lesion detection module that detects and/or segments hotspots throughout an entire body; and (B) a prostate lesion module that detects and/or segments hotspots within the prostate. In certain embodiments, the instructions cause the processor to generate the hotspot list and/or maps using each of (A) and (B) and merge the results.

In certain embodiments, at step (d) the instructions cause the processor to segment and classify the set of one or more hotspots to create a labeled 3D hotspot map that identifies, for each hotspot, a corresponding 3D hotspot volume within the 3D functional image, and in which each hotspot is labeled as belonging to a particular hotspot class of a plurality of hotspot classes [e.g., each hotspot class identifying a particular anatomical and/or tissue region (e.g., lymph, bone, prostate) that a lesion represented by the hotspot is determined to be located] by: using a first machine learning module to segment a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class [e.g., identifying all hotspots as belonging to a single hotspot class, so as to differentiate between background regions and hotspot volumes (e.g., but not between different types of hotspots) (e.g., such that each hotspot volume identified by the first 3D hotspot map is labeled as belonging to a single hotspot class, as opposed to background)]; using a second machine learning module to segment a second initial set of one or more hotspots within the 3D functional image, thereby creating a second initial 3D hotspot map that identifies a second set of initial hotspot volumes, wherein the second machine learning module segments the 3D functional image to according to the plurality of different hotspot classes, such that the second initial 3D hotspot map is a multi-class 3D hotspot map in which each hotspot volume is labeled as belonging to a particular one of the plurality of different hotspot classes (e.g., so as to differentiate between hotspot volumes corresponding to different hotspot classes, as well between hotspot volumes and as background regions); and merging the first initial 3D hotspot map and the second initial 3D hotspot map by, of at least a portion of the hotspot volumes identified by the first initial 3D hotspot map: identifying a matching hotspot volume of the second initial 3D hotspot map (e.g., by identifying substantially overlapping hotspot volumes of the first and second initial 3D hotspot maps), the matching hotspot volume of the second 3D hotspot map having been labeled as belonging to a particular hotspot class of the plurality of different hotspot classes; and labeling the particular hotspot volume of the first initial 3D hotspot map as belonging to the particular hotspot class (that the matching hotspot volume is labeled as belonging to), thereby creating a merged 3D hotspot map that includes segmented hotspot volumes of the first 3D hotspot map having been labeled according classes that matching hotspots of the second 3D hotspot map are identified as belonging to; and at step (e) the instructions cause the processor to store and/or provide, for display and/or further processing, the merged 3D hotspot map.

In certain embodiments, the plurality of different hotspot classes comprise one or more members selected from the group consisting of: (i) bone hotspots, determined (e.g., by the second machine learning module) to represent lesions located in bone, (ii) lymph hotspots, determined (e.g., by the second machine learning module) to represent lesions located in lymph nodes, and (iii) prostate hotspots, determined (e.g., by the second machine learning module) to represent lesions located in a prostate.

In certain embodiments, the instructions further cause the processor to: (f)
receive and/or access the hotspot list; and (g) for each hotspot in the hotspot list, segment the hotspot using an analytical model [e.g., thereby creating a 3D map of analytically segmented hotspots (e.g., the 3D map identifying, for each hotspot, a hotspot volume comprising voxels of the 3D anatomical image and/or functional image enclosed by the segmented hotspot region)].

In certain embodiments, the instructions further cause the processor to: (h) receive and/or access the hotspot map; and (i) for each hotspot in the hotspot map, segment the hotspot using an analytical model [e.g., thereby creating a 3D map of analytically segmented hotspots (e.g., the 3D map identifying, for each hotspot, a hotspot volume comprising voxels of the 3D anatomical image and/or functional image enclosed by the segmented hotspot region)].

In certain embodiments, the analytical model is an adaptive thresholding method, and at step (i), the instructions cause the processor to: determine one or more reference values, each based on a measure of intensities of voxels of the 3D functional image located within a particular reference volume corresponding to a particular reference tissue region (e.g., a blood pool reference value determined based on intensities within an aorta volume corresponding to a portion of an aorta of a subject; e.g., a liver reference value determined based on intensities within a liver volume corresponding to a liver of a subject); and for each particular hotspot volume of the 3D hotspot map: determine a corresponding hotspot intensity based on intensities of voxels within the particular hotspot volume [e.g., wherein the hotspot intensity is a maximum of intensities (e.g., representing SUVs) of voxels within the particular hotspot volume]; and determine a hotspot-specific threshold value for the particular hotspot based on (i) the corresponding hotspot intensity and (ii) at least one of the one or more reference value(s).

In certain embodiments, the hotspot-specific threshold value is determined using a particular threshold function selected from a plurality of threshold functions, the particular threshold function selected based a comparison of the corresponding hotspot intensity with the at least one reference value [e.g., wherein each of the plurality of threshold functions is associated with a particular range of intensity (e.g., SUV) values, and the particular threshold function is selected according to the particular range that the hotspot intensity and/or a (e.g., predetermined) percentage thereof falls within (e.g., and wherein each particular range of intensity values is bounded at least in part by a multiple of the at least one reference value)].

In certain embodiments, the hotspot-specific threshold value is determined (e.g., by the particular threshold function) as a variable percentage of the corresponding hotspot intensity, wherein the variable percentage decreases with increasing hotspot intensity [e.g., wherein the variable percentage is itself a function (e.g., a decreasing function) of the corresponding hotspot intensity].

In another aspect, the invention is directed to a system for automatically processing 3D images of a subject to identify and/or characterize (e.g., grade; e.g., classify, e.g., as representing a particular lesion type) cancerous lesions within the subject, the system comprising: a processor of a computing device; and a memory having instructions stored thereon, wherein the instructions, when executed by the processor, cause the processor to: (a) receive (e.g., and/or access) a 3D functional image [e.g., positron emission tomography (PET); e.g., single-photon emission computed tomography (SPECT)] of the subject obtained using a functional imaging modality; (b) automatically segment, using a first machine learning module, a first initial set of one or more hotspots within the 3D functional image, thereby creating a first initial 3D hotspot map that identifies a first set of initial hotspot volumes, a corresponding 3D hotspot volume within the 3D functional image, wherein the first machine learning module segments hotspots of the 3D functional image according to a single hotspot class [e.g., identifying all hotspots as belonging to a single hotspot class, so as to differentiate between background regions and hotspot volumes (e.g., but not between different types of hotspots) (e.g., such that each hotspot volume identified by the first 3D hotspot map is labeled as belonging to a single hotspot class, as opposed to background)]; (c) automatically segment, using a second machine learning module, a second initial set of one or more hotspots within the 3D functional image, thereby creating a second initial 3D hotspot map that identifies a second set of initial hotspot volumes, wherein the second machine learning module segments the 3D functional image to according to a plurality of different hotspot classes [e.g., each hotspot class identifying a particular anatomical and/or tissue region (e.g., lymph, bone, prostate) that a lesion represented by the hotspot is determined to be located], such that the second initial 3D hotspot map is a multi-class 3D hotspot map in which each hotspot volume is labeled as belonging to a particular one of the plurality of different hotspot classes (e.g., so as to differentiate between hotspot volumes corresponding to different hotspot classes, as well between hotspot volumes and as background regions); (d) merge the first initial 3D hotspot map and the second initial 3D hotspot map by, for each particular hotspot volume of at least a portion of the first set of initial hotspot volumes identified by the first initial 3D hotspot map: identifying a matching hotspot volume of the second initial 3D hotspot map (e.g., by identifying substantially overlapping hotspot volumes of the first and second initial 3D hotspot maps), the matching hotspot volume of the second 3D hotspot map having been labeled as belonging to a particular hotspot class of the plurality of different hotspot classes; and labeling the particular hotspot volume of the first initial 3D hotspot map as belonging to the particular hotspot class (that the matching hotspot volume is labeled as belonging to), thereby creating a merged 3D hotspot map that includes segmented hotspot volumes of the first 3D hotspot map having been labeled according classes that matching hotspots of the second 3D hotspot map are identified as belonging to; and (e) store and/or provide, for display and/or further processing, the merged 3D hotspot map.

In certain embodiments, the plurality of different hotspot classes comprises one or more members selected from the group consisting of: (i) bone hotspots, determined (e.g., by the second machine learning module) to represent lesions located in bone, (ii) lymph hotspots, determined (e.g., by the second machine learning module) to represent lesions located in lymph nodes, and (iii) prostate hotspots, determined (e.g., by the second machine learning module) to represent lesions located in a prostate.

In another aspect, the invention is directed to a system for automatically processing 3D images of a subject to identify and/or characterize e.g., grade; e.g., classify, e.g., as representing a particular lesion type) cancerous lesions within the subject, via an adaptive thresholding approach the system comprising: a processor of a computing device; and a memory having instructions stored thereon, wherein the instructions, when executed by the processor, cause the processor to: (a) receive (e.g., and/or access) a 3D functional image [e.g., positron emission tomography (PET); e.g., single-photon emission computed tomography (SPECT)] of the subject obtained using a functional imaging modality; (b) receive (e.g., and/or access) a preliminary 3D hotspot map identifying, within the 3D functional image, one or more preliminary hotspot volumes; (c) determine one or more reference values, each based on a measure of intensities of voxels of the 3D functional image located within a particular reference volume corresponding to a particular reference tissue region (e.g., a blood pool reference value determined based on intensities within an aorta volume corresponding to a portion of an aorta of a subject; e.g., a liver reference value determined based on intensities within a liver volume corresponding to a liver of a subject); (d) create a refined 3D hotspot map based on the preliminary hotspot volumes and using an adaptive threshold-based segmentation by, for each particular preliminary hotspot volume of at least a portion of the one or more preliminary hotspot volumes identified by the preliminary 3D hotspot map: determining a corresponding hotspot intensity based on intensities of voxels within the particular preliminary hotspot volume [e.g., wherein the hotspot intensity is a maximum of intensities (e.g., representing SUVs) of voxels within the particular preliminary hotspot volume]; and determining a hotspot-specific threshold value for the particular preliminary hotspot based on (i) the corresponding hotspot intensity and (ii) at least one of the one or more reference value(s); segmenting at least a portion of the 3D functional (e.g., a sub-volume about in the particular preliminary hotspot volume) using a threshold-based segmentation algorithm that performs image segmentation using the hotspot-specific threshold value determined for the particular preliminary hotspot [e.g., and identifies clusters of voxels (e.g., 3D clusters of voxels connected to each other in an n-connected component fashion (e.g., where ₙ = 6, ₙ = 18, etc.)) having intensities above the hotspot-specific threshold value and comprising a maximum intensity voxel of the preliminary hotspot], thereby determining a refined, analytically segmented, hotspot volume corresponding to the particular preliminary hotspot volume; and including the refined hotspot volume in the refined 3D hotspot map; and (e) store and/or provide, for display and/or further processing, the refined 3D hotspot map.

In certain embodiments, the hotspot-specific threshold value is determined using a particular threshold function selected from a plurality of threshold functions, the particular threshold function selected based a comparison of the corresponding hotspot intensity with the at least one reference value [e.g., wherein each of the plurality of threshold functions is associated with a particular range of intensity (e.g., SUV) values, and the particular threshold function is selected according to the particular range that the hotspot intensity and/or a (e.g., predetermined) percentage thereof falls within (e.g., and wherein each particular range of intensity values is bounded at least in part by a multiple of the at least one reference value)].

In certain embodiments, the hotspot-specific threshold value is determined (e.g., by the particular threshold function) as a variable percentage of the corresponding hotspot intensity, wherein the variable percentage decreases with increasing hotspot intensity [e.g., wherein the variable percentage is itself a function (e.g., a decreasing function) of the corresponding hotspot intensity].

In another aspect, the invention is directed to a system for automatically processing 3D images of a subject to identify and/or characterize (e.g., grade; e.g., classify, e.g., as representing a particular lesion type) cancerous lesions within the subject, the system comprising: a processor of a computing device; a memory having instructions stored thereon, wherein the instructions, when executed by the processor, cause the processor to: (a) receive (e.g., and/or access) a 3D anatomical image of the subject obtained using an anatomical imaging modality [e.g., x-ray computed tomography (CT); e.g., magnetic resonance imaging (MRI); e.g., ultra-sound], wherein the 3D anatomical image comprises a graphical representation of tissue (e.g., soft-tissue and/or bone) within the subject; (b) automatically segment the 3D anatomical image to create a 3D segmentation map that identifies a plurality of volumes of interest (VOIs) in the 3D anatomical image, including a liver volume corresponding to a liver of the subject and an aorta volume corresponding to an aorta portion (e.g., thoracic and/or abdominal portion); (c) receive (e.g., and/or access) a 3D functional image of the subject obtained using a functional imaging modality [e.g., positron emission tomography (PET); e.g., single-photon emission computed tomography (SPECT)][e.g., wherein the 3D functional image comprises a plurality of voxels, each representing a particular physical volume within the subject and having an intensity value that represents detected radiation emitted from the particular physical volume, wherein at least a portion of the plurality of voxels of the 3D functional image represent physical volumes within the target tissue region]; (d) automatically segment one or more hotspots within the 3D functional image, each segmented hotspot corresponding to a local region of elevated intensity with respect to its surrounding and representing (e.g., indicative of) a potential cancerous lesion within the subject, thereby identifying one or more automatically segmented hotspot volumes; (e) causing rendering of a graphical representation of the one or more automatically segmented hotspot volumes for display within an interactive graphical user interface (GUI) (e.g., a quality control and reporting GUI); (f) receive, via the interactive GUI, a user selection of a final hotspot set comprising at least a portion (e.g., up to all) of the one or more automatically segmented hotspot volumes; (g) determine, for each hotspot volume of the final set, a lesion index value based on (i) intensities of voxels of the functional image corresponding to (e.g., located within) the hotspot volume and (ii) one or more reference values determined using intensities of voxels of the functional image corresponding to the liver volume and the aorta volume; and (e) store and/or provide for display and/or further processing, the final hotspot set and/or lesion index values.

In certain embodiments, at step (b) the instructions cause the processor to segment the anatomical image, such that the 3D segmentation map identifies one or more bone volumes corresponding to one or more bones of the subject, and at step (d) the instructions cause the processor to identify, within the functional image, a skeletal volume using the one or more bone volumes and segmenting one or more bone hotspot volumes located within the skeletal volume (e.g., by applying one or more difference of Gaussian filters and thresholding the skeletal volume).

In certain embodiments, at step (b) the instructions cause the processor to segment the anatomical image such that the 3D segmentation map identifies one or more organ volumes corresponding to soft-tissue organs of the subject (e.g., left/right lungs, left/right gluteus maximus, urinary bladder, liver, left/right kidney, gallbladder, spleen, thoracic and abdominal aorta and, optionally (e.g., for patients not having undergone radical prostatectomy, a prostate), and at step (d) the instructions cause the processor to identify, within the functional image, a soft tissue (e.g., a lymph and, optionally, prostate) volume using the one or more segmented organ volumes and segmenting one or more lymph and/or prostate hotspot volumes located within the soft tissue volume (e.g., by applying one or more Laplacian of Gaussian filters and thresholding the soft-tissue volume).

In certain embodiments, at step (d) the instructions cause the processor to, prior to segmenting the one or more lymph and/or prostate hotspot volumes, adjust intensities of the functional image to suppress intensity from one or more high-uptake tissue regions (e.g., using one or more suppression methods described herein).

In certain embodiments, at step (g) the instructions cause the processor to determine a liver reference value using intensities of voxels of the functional image corresponding to the liver volume.

In certain embodiments, the instructions cause the processor to: fit a two component Gaussian mixture model two a histogram of intensities of functional image voxels corresponding to the liver volume, use the two-component Gaussian mixture model fit to identify and exclude voxels having intensities associated with regions of abnormally low uptake from the liver volume, and determine the liver reference value using intensities of remaining (e.g., not excluded) voxels.

Features of embodiments described with respect to one aspect of the invention may be applied with respect to another aspect of the invention.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1A is a block flow diagram of an example process for artificial intelligence (AI) -based lesion detection, according to an illustrative embodiment.

FIG. 1B is a block flow diagram of an example process for AI-based lesion detection, according to an illustrative embodiment.

FIG. 1C is a block flow diagram of an example process for AI-based lesion detection, according to an illustrative embodiment.

FIG. 2A is a graph showing a histogram of liver SUV values overlaid with a two-component Gaussian mixture model, according to an illustrative embodiment.

FIG. 2B is a PET image overlaid on a CT images showing a portion of a liver volume used for calculation of a liver reference value, according to an illustrative embodiment.

FIG. 2C is a block flow diagram of an example process for computing reference intensity values that avoids / reduces impact from tissue regions associated with low radiopharmaceutical uptake, according to an illustrative embodiment.

FIG. 3 is a block flow diagram of an example process for correcting for intensity bleed from one or more tissue regions associated with high radiopharmaceutical uptake, according to an illustrative embodiment.

FIG. 4 is block flow diagram of an example process for anatomically labeling hotspots corresponding to detected lesions, according to an illustrative embodiment.

FIG. 5A is a block flow diagram of an example process for interactive lesion detection, allowing for user feedback and review via a graphical user interface (GUI), according to an illustrative embodiment.

FIG. 5B is an example process for user review, quality control, and reporting of automatically detected lesions, according to an illustrative embodiment.

FIG. 6A is a screenshot of a GUI used for confirming accurate segmentation of a liver reference volume, according to an illustrative embodiment.

FIG. 6B is a screenshot of a GUI used for confirming accurate segmentation of an aorta portion (blood pool) reference volume, according to an illustrative embodiment.

FIG. 6C is a screenshot of a GUI used for user selection and/or validation of automatically segmented hotspots corresponding to detected lesions within a subject, according to an illustrative embodiment.

FIG. 6D is a screenshot of a portion of a GUI allowing a user to manually identify lesions within an image, according to an illustrative embodiment.

FIG. 6E is a screenshot of another portion of a GUI allowing a user to manually identify lesions within an image, according to an illustrative embodiment.

FIG. 7 is a screenshot of a portion of a GUI showing a quality control checklist, according to an illustrative embodiment.

FIG. 8 is a screenshot of a report generated by a user, using an embodiment of the automated lesion detection tools described herein, according to an illustrative embodiment.

FIG. 9 is a block flow diagram showing an example architecture for hotspot (lesion) segmentation via a machine learning module that receives a 3D anatomical image, a 3D functional image, and a 3D segmentation map as input, according to an illustrative embodiment.

FIG. 10A is a block flow diagram showing an example process wherein lesion type mapping is performed following hotpot segmentation, according to an illustrative embodiment.

FIG. 10B is another block flow diagram showing an example process wherein lesion type mapping is performed following hotpot segmentation, illustrating use of a 3D segmentation map, according to an illustrative embodiment.

FIG. 11A is a block flow diagram showing a process for detecting and/or segmenting hotspots representing lesions using a full-body network and a prostate-specific network, according to an illustrative embodiment.

FIG. 11B is a block flow diagram showing a process for detecting and/or segmenting hotspots representing lesions using a full-body network and a prostate-specific network, according to an illustrative embodiment.

FIG. 12 is a block flow diagram showing use of an analytical segmentation step following AI-based hotspot segmentation, according to an illustrative embodiment.

FIG. 13A is a block diagram showing an example U-net architecture used for hotspot segmentation, according to an illustrative embodiment.

FIG. 13B and FIG. 13C are a block diagrams showing an example FPN architectures for hotspot segmentation, according to an illustrative embodiment.

FIG. 14A, FIG. 14B, and FIG. 14C show example images demonstrating segmentation of hotspots using a U-net architecture, according to an illustrative embodiment.

FIG. 15A, and FIG. 15B show example images demonstrating segmentation of hotspots using a FPN architecture, according to an illustrative embodiment.

FIG. 16A, FIG. 16B, FIG. 16C, FIG. 16D, and FIG. 16E are screenshots of an example GUI for uploading, analyzing, and generating a report from medical image data, according to an illustrative embodiment.

FIG. 17A and FIG. 17B are block flow diagrams of example processes for segmenting and classifying hotspots using two parallel machine learning modules, according to an illustrative embodiment.

FIG. 17C is a block flow diagram illustrating interaction and data flow between various software modules (e.g., APIs) of an example implementation of a process for segmenting and classifying hotspots using two parallel machine learning modules, according to an illustrative embodiment.

FIG. 18A is a block flow diagram of an example process for segmenting hotspots by an analytical model that uses an adaptive thresholding method, according to an illustrative embodiment.

FIG. 18B and FIG. 18C are graphs showing variation in a hotspot-specific threshold used in an adaptive thresholding method, as a function of hotspot intensity (SUVₘₐₓ), according to an illustrative embodiment.

FIG. 18D, FIG. 18E, and FIG. 18F are diagrams illustrating certain thresholding techniques, according to illustrative embodiments.

FIG. 18G is a diagram showing intensities of prostate voxels along axial, sagittal, and coronal planes, along with a histogram of prostate voxel intensity values and an illustrative setting of a threshold scaling factor, according to an illustrative embodiment.

FIG. 19A is a block flow diagram illustrating hotspot segmentation using a conventional manual ROI definition and conventional fixed and/or relative thresholding, according to an illustrative embodiment.

FIG. 19B is a block flow diagram illustrating hotspot segmentation using an AI-based approach in combination with an adaptive thresholding method, according to an illustrative embodiment.

FIG. 20 is a set of images comparing example segmentation results for thresholding alone with segmentation results obtained via an AI-based approach in combination with an adaptive thresholding method, according to an illustrative embodiment.

FIG. 21A, FIG. 21B, FIG. 21C, FIG. 21D, FIG. 21E, FIG. 21F, FIG. 21G, FIG. 21H, and FIG. 21I show a series of 2D slices of a 3D PET image, moving along a vertical direction in an abdominal region. The images compare hotspot segmentation results within an abdominal region performed by a thresholding method alone (left hand images) with those of a machine learning approach in accordance with certain embodiments described herein (right hand images), and show hotspot regions identified by each method overlaid on the PET image slices.

FIG. 22 is a block flow diagram of a process for uploading and analyzing PET/CT image data using a CAD device providing for automated image analysis according to certain embodiments described herein.

FIG. 23 is a screenshot of an example GUI allowing users to upload image data for review and analysis via a CAD device providing for automated image analysis according to certain embodiments described herein.

FIG. 24 is a screenshot of an example GUI viewer allowing a user to review and analyze medical image data (e.g., 3D PET/CT images) and results of automated image analysis, according to an illustrative embodiment.

FIG. 25 is a screenshot of an automatically generated report, according to an illustrative embodiment.

FIG. 26 is a block flow diagram of an example workflow for analysis of medical image data providing for automated analysis along with user input and review, according to an illustrative embodiment.

FIG. 27 shows three views of a CT image with segmented bone and soft-tissue volumes overlaid, according to an illustrative embodiment.

FIG. 28 is a block flow diagram of an analytical model for segmenting hotspots, according to an illustrative embodiment.

FIG. 29A is a block diagram of a cloud computing architecture, used in certain embodiments.

FIG. 29B is a block diagram of an example microservice communication flow, used in certain embodiments.

FIG. 30 is a block diagram of an exemplary cloud computing environment, used in certain embodiments.

FIG. 31 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION

It is contemplated that systems, devices, methods, and processes of the claimed invention encompass variations and adaptations developed using information from the embodiments described herein. Adaptation and/or modification of the systems, devices, methods, and processes described herein may be performed by those of ordinary skill in the relevant art.

Throughout the description, where articles, devices, and systems are described as having, including, or comprising specific components, or where processes and methods are described as having, including, or comprising specific steps, it is contemplated that, additionally, there are articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader - the presence and/or placement of a header is not intended to limit the scope of the subject matter described herein.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

In certain embodiments, the term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

### A. Nuclear Medicine Images

Nuclear medicine images are obtained using a nuclear imaging modality such as bone scan imaging, Positron Emission Tomography (PET) imaging, and Single-Photon Emission Tomography (SPECT) imaging.

As used herein, an “image” - for example, a 3-D image of mammal - includes any visual representation, such as a photo, a video frame, streaming video, as well as any electronic, digital or mathematical analogue of a photo, video frame, or streaming video. Any apparatus described herein, in certain embodiments, includes a display for displaying an image or any other result produced by the processor. Any method described herein, in certain embodiments, includes a step of displaying an image or any other result produced via the method.

As used herein, “3-D” or “three-dimensional” with reference to an “image” means conveying information about three dimensions. A 3-D image may be rendered as a dataset in three dimensions and/or may be displayed as a set of two-dimensional representations, or as a three-dimensional representation.

In certain embodiments, nuclear medicine images use imaging agents comprising radiopharmaceuticals. Nuclear medicine images are obtained following administration of a radiopharmaceutical to a patient (e.g., a human subject), and provide information regarding the distribution of the radiopharmaceutical within the patient. Radiopharmaceuticals are compounds that comprise a radionuclide.

As used herein, “administering” an agent means introducing a substance (e.g., an imaging agent) into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments

As used herein, “radionuclide” refers to a moiety comprising a radioactive isotope of at least one element. Exemplary suitable radionuclides include but are not limited to those described herein. In some embodiments, a radionuclide is one used in positron emission tomography (PET). In some embodiments, a radionuclide is one used in single-photon emission computed tomography (SPECT). In some embodiments, a non-limiting list of radionuclides includes ^{99m}Tc, ¹¹¹In, ⁶⁴Cu, ⁶⁷Ga, ⁶⁸Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹²⁶I, ¹³¹I, ¹¹C, ¹³N, ¹⁵O, ¹⁸F, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹⁰⁵Rh, ¹¹¹Ag, ⁸⁹Zr, ²²⁵Ac, ⁸²Rb, ⁷⁵Br, ⁷⁶Br, ⁷⁷Br, ⁸⁰Br, ^{80m}Br, ⁸²Br, ⁸³Br, ²¹¹At and ¹⁹²Ir.

As used herein, the term “radiopharmaceutical” refers to a compound comprising a radionuclide. In certain embodiments, radiopharmaceuticals are used for diagnostic and/or therapeutic purposes. In certain embodiments, radiopharmaceuticals include small molecules that are labeled with one or more radionuclide(s), antibodies that are labeled with one or more radionuclide(s), and antigen-binding portions of antibodies that are labeled with one or more radionuclide(s).

Nuclear medicine images (e.g., PET scans; e.g., SPECT scans; e.g., whole-body bone scans; e.g. composite PET-CT images; e.g., composite SPECT-CT images) detect radiation emitted from the radionuclides of radiopharmaceuticals to form an image. The distribution of a particular radiopharmaceutical within a patient may be determined by biological mechanisms such as blood flow or perfusion, as well as by specific enzymatic or receptor binding interactions. Different radiopharmaceuticals may be designed to take advantage of different biological mechanisms and/or particular specific enzymatic or receptor binding interactions and thus, when administered to a patient, selectively concentrate within particular types of tissue and/or regions within the patient. Greater amounts of radiation are emitted from regions within the patient that have higher concentrations of radiopharmaceutical than other regions, such that these regions appear brighter in nuclear medicine images. Accordingly, intensity variations within a nuclear medicine image can be used to map the distribution of radiopharmaceutical within the patient. This mapped distribution of radiopharmaceutical within the patient can be used to, for example, infer the presence of cancerous tissue within various regions of the patient’s body.

For example, upon administration to a patient, technetium 99m methylenediphosphonate (^{99m}Tc MDP) selectively accumulates within the skeletal region of the patient, in particular at sites with abnormal osteogenesis associated with malignant bone lesions. The selective concentration of radiopharmaceutical at these sites produces identifiable hotspots - localized regions of high intensity in nuclear medicine images. Accordingly, presence of malignant bone lesions associated with metastatic prostate cancer can be inferred by identifying such hotspots within a whole-body scan of the patient. As described in the following, risk indices that correlate with patient overall survival and other prognostic metrics indicative of disease state, progression, treatment efficacy, and the like, can be computed based on automated analysis of intensity variations in whole-body scans obtained following administration of ⁹⁹mTc MDP to a patient. In certain embodiments, other radiopharmaceuticals can also be used in a similar fashion to ⁹⁹mTc MDP.

In certain embodiments, the particular radiopharmaceutical used depends on the particular nuclear medicine imaging modality used. For example 18F sodium fluoride (NaF) also accumulates in bone lesions, similar to ⁹⁹mTc MDP, but can be used with PET imaging. In certain embodiments, PET imaging may also utilize a radioactive form of the vitamin choline, which is readily absorbed by prostate cancer cells.

In certain embodiments, radiopharmaceuticals that selectively bind to particular proteins or receptors of interest - particularly those whose expression is increased in cancerous tissue may be used. Such proteins or receptors of interest include, but are not limited to tumor antigens, such as CEA, which is expressed in colorectal carcinomas, Her2/neu, which is expressed in multiple cancers, BRCA 1 and BRCA 2, expressed in breast and ovarian cancers; and TRP-1 and -2, expressed in melanoma.

For example, human prostate-specific membrane antigen (PSMA) is upregulated in prostate cancer, including metastatic disease. PSMA is expressed by virtually all prostate cancers and its expression is further increased in poorly differentiated, metastatic and hormone refractory carcinomas. Accordingly, radiopharmaceuticals corresponding to PSMA binding agents (e.g., compounds that a high affinity to PSMA) labelled with one or more radionuclide(s) can be used to obtain nuclear medicine images of a patient from which the presence and/or state of prostate cancer within a variety of regions (e.g., including, but not limited to skeletal regions) of the patient can be assessed. In certain embodiments, nuclear medicine images obtained using PSMA binding agents are used to identify the presence of cancerous tissue within the prostate, when the disease is in a localized state. In certain embodiments, nuclear medicine images obtained using radiopharmaceuticals comprising PSMA binding agents are used to identify the presence of cancerous tissue within a variety of regions that include not only the prostate, but also other organs and tissue regions such as lungs, lymph nodes, and bones, as is relevant when the disease is metastatic.

In particular, upon administration to a patient, radionuclide labelled PSMA binding agents selectively accumulate within cancerous tissue, based on their affinity to PSMA. In a similar manner to that described above with regard to ^{99m}Tc MDP, the selective concentration of radionuclide labelled PSMA binding agents at particular sites within the patient produces detectable hotspots in nuclear medicine images. As PSMA binding agents concentrate within a variety of cancerous tissues and regions of the body expressing PSMA, localized cancer within a prostate of the patient and/or metastatic cancer in various regions of the patient’s body can be detected, and evaluated. Risk indices that correlate with patient overall survival and other prognostic metrics indicative of disease state, progression, treatment efficacy, and the like, can be computed based on automated analysis of intensity variations in nuclear medicine images obtained following administration of a PSMA binding agent radiopharmaceutical to a patient.

A variety of radionuclide labelled PSMA binding agents may be used as radiopharmaceutical imaging agents for nuclear medicine imaging to detect and evaluate prostate cancer. In certain embodiments, the particular radionuclide labelled PSMA binding agent that is used depends on factors such as the particular imaging modality (e.g., PET; e.g., SPECT) and the particular regions (e.g., organs) of the patient to be imaged. For example, certain radionuclide labelled PSMA binding agents are suited for PET imaging, while others are suited for SPECT imaging. For example, certain radionuclide labelled PSMA binding agents facilitate imaging a prostate of the patient, and are used primarily when the disease is localized, while others facilitate imaging organs and regions throughout the patient’s body, and are useful for evaluating metastatic prostate cancer.

A variety of PSMA binding agents and radionuclide labelled versions thereof are described in U.S. Pat. Nos. 8,778,305, 8,211,401, and 8,962,799, each of which are incorporated herein by reference in their entireties. Several PSMA binding agents and radionuclide labelled versions thereof are also described in PCT Application PCT/US2017/058418, filed Oct. 26, 2017 (PCT publication WO 2018/081354), the content of which is incorporated herein by reference in its entirety. Section J, below, describes several example PSMA binding agents and radionuclide labelled versions thereof, as well.

### B. Automated Lesion Detection and Analysis

### I. Automated Lesion Detection

In certain embodiments, the systems and methods described herein utilize machine learning techniques for automated image segmentation and detection of hotspots corresponding to and indicative of possible cancerous lesions within a subject.

In certain embodiments, the systems and methods described herein may be implemented in a cloud-based platform, for example as described in PCT/US2017/058418, filed Oct. 26, 2017 (PCT publication WO 2018/081354), the content of which is hereby incorporated by reference in its entirety.

In certain embodiments, as described herein, machine learning modules implement one or more machine learning techniques, such as random forest classifiers, artificial neural networks (ANNs), convolutional neural networks (CNNs), and the like. In certain embodiments, machine learning modules implementing machine learning techniques are trained, for example using manually segmented and/or labeled images, to identify and/or classify portions of images. Such training may be used to determine various parameters of machine learning algorithms implemented by a machine learning module, such as weights associated with layers in neural networks. In certain embodiments, once a machine learning module is trained, e.g., to accomplish a specific task such as identifying certain target regions within images, values of determined parameters are fixed and the (e.g., unchanging, static) machine learning module is used to process new data (e.g., different from the training data) and accomplish its trained task without further updates to its parameters (e.g., the machine learning module does not receive feedback and/or update). In certain embodiments, machine learning modules may receive feedback, e.g., based on user review of accuracy, and such feedback may be used as additional training data, to dynamically update the machine learning module. In some embodiments, the trained machine learning module is a classification algorithm with adjustable and/or fixed (e.g., locked) parameters, e.g., a random forest classifier.

In certain embodiments, machine learning techniques are used to automatically segment anatomical structures in anatomical images, such as CT, MRI, ultra-sound, etc. images, in order to identify volumes of interest corresponding to specific target tissue regions such as specific organs (e.g., a prostate, lymph node regions, a kidney, a liver, a bladder, an aorta portion) as well as bones. In this manner, machine learning modules may be used to generate segmentation masks and/or segmentation maps (e.g., comprising a plurality of segmentation masks, each corresponding to and identifying a particular target tissue region) that can be mapped to (e.g., projected onto) functional images, such as PET or SPECT images, to provide anatomical context for evaluating intensity fluctuations therein. Approaches for segmenting images and using the obtained anatomical context for analysis of nuclear medicine images are described, for example, in further detail in PCT/US2019/012486, filed Jan. 7, 2019 (PCT publication WO 2019/136349) and PCT/EP2020/050132, filed Jan. 6, 2020 (PCT publication WO 2020/144134), the contents of each of which is hereby incorporated by reference in their entirety.

In certain embodiments, potential lesions are detected as regions of locally high intensity in functional images, such as PET images. These localized regions of elevated intensity, also referred to as hotspots, can be detected using image processing techniques not necessarily involving machine learning, such as filtering and thresholding, and segmented using approaches such as the fast marching method. Anatomical information established from the segmentation of anatomical images allows for anatomical labeling of detected hotspots representing potential lesions. Anatomical context may also be useful in allowing different detection and segmentation techniques to be used for hotspot detection in different anatomical regions, which can increase sensitivity and performance.

In certain embodiments, automatically detected hotspots may be presented to a user via an interactive graphical user interface (GUI). In certain embodiments, to account for target lesions detected by the user (e.g., physician), but that are missed or poorly segmented by the system, a manual segmentation tool is included in the GUI, allowing the user to manually “paint” regions of images that they perceive as corresponding to lesions of any shape and size. These manually segmented lesions may then be included, along with selected automatically detected target lesions, in subsequently generated reports.

### II. AI-Based Lesion Detection

In certain embodiments, the systems and methods described herein utilize one or more machine learning modules to analyze intensities of 3D functional images and detect hotspots representing potential lesions. For example, by collecting a dataset of PET/CT images in which hotspots that represent lesions have been manually detected and segmented, training material for AI-based lesion detection algorithms can be obtained. These manually labeled images can be used to train one or more machine learning algorithms to automatically analyze functional images (e.g., PET images) to accurately detect and segment hotspots corresponding to cancerous lesions.

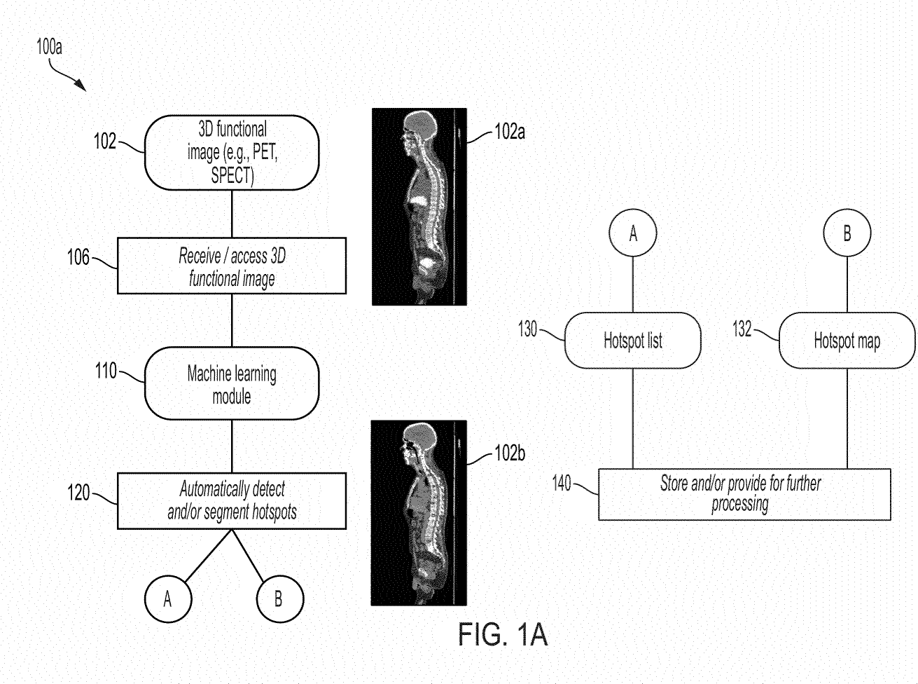

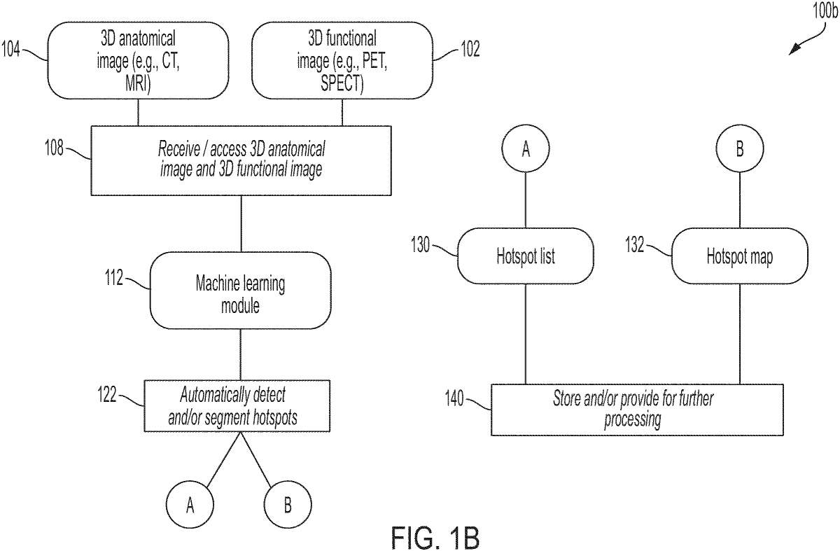

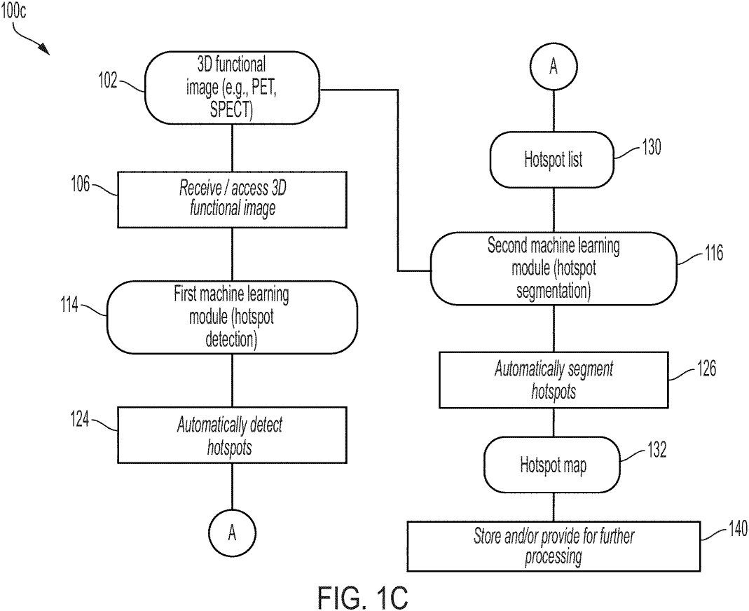

FIG. 1A shows an example process 100a for automated lesion detection and/or segmentation using machine learning modules that implement machine learning algorithms, such as ANNs, CNNs, and the like. As shown in FIG. 1A, a 3D functional image 102, such as a PET or SPECT image, is received 106 and used as input to a machine learning module 110. FIG. 1A shows an example PET image, obtained using PyL™ as a radiopharmaceutical 102a. The PET image 102a is shown overlaid on a CT image (e.g., as a PET/CT image), but the machine learning module 110 may receive the PET (e.g., or other functional image) itself (e.g., not including the CT, or other anatomical image) as input. In certain embodiments, as described below, an anatomical image may also be received as input. The machine learning module automatically detects and/or segments hotspots 120 determined (by the machine learning module) to represent potential cancerous lesions. An example image showing hotspots appearing in a PET image 120b is shown in FIG. 1A as well. Accordingly, the machine learning module generates, as output, one or both of (i) a hotspot list 130 and (ii) a hotspot map 132. In certain embodiments, the hotspot list identifies locations (e.g., centers of mass) of the detected hotspots. In certain embodiments, the hotspot map is identifies 3D volumes and/or delineates 3D boundaries of detected hotspots, as determined via image segmentation performed by the machine learning module 110. The hotspot list and/or hotspot map may be stored and/or provided (e.g., to other software modules) for display and/or further processing 140.

In certain embodiments, machine learning-based lesion detection algorithms may be trained on, and utilize, not only functional image information (e.g., from a PET image), but also anatomical information. For example, in certain embodiments, one or more machine learning modules used for lesion detection and segmentation may be trained on, and receive as input, two channels - a first channel corresponding to a portion of a PET image, and a second channel corresponding to a portion of a CT image. In certain embodiments, information derived from an anatomical (e.g., CT) image may also be used as input to machine learning modules for lesion detection and/or segmentation. For example, in certain embodiments, 3D segmentation maps identifying various tissue regions within an anatomical and/or functional image can also be used (e.g., received as input, e.g., as separate input channel, by one or more machine learning modules) to provide anatomical context.

FIG. 1B shows an example process 100b in which both a 3D anatomical image 104, such as a CT or MR image, and a 3D functional image 102 are received 108 and used as input to a machine learning module 112 that performs hotspot detection and/or segmentation 122 based on information (e.g., voxel intensities) from both the 3D anatomical image 104 and the 3D functional image 102 as described herein. A hotspot list 130 and/or hotspot map 132 may be generated as output from the machine learning module, and stored / provided for further processing (e.g., graphical rendering for display, subsequent operations by other software modules, etc.) 140.

In certain embodiments, automated lesion detection and analysis (e.g., for inclusion in a report) includes three tasks: (i) detection of hotspots corresponding to lesions, (ii) segmentation of detected hotspots (e.g., to identify, within a functional image, a 3D volume corresponding to each lesion), and (iii) classification of detected hotspots as having high or low probability of corresponding to a true lesion within the subject (e.g., and thus appropriate for inclusion in a radiologist report or not). In certain embodiments, one or more machine learning modules may be used to accomplish these three tasks, e.g., one by one (e.g., in sequence) or in combination. For example, in certain embodiments, a first machine learning module is trained to detect hotspots and identify hotspot locations, a second machine learning module is trained to segment hotspots, and a third machine learning module is trained to classify detected hotspots, for example using information obtained from the other two machine learning modules.

For example, as shown in the example process 100c of FIG. 1C, a 3D functional image 102 may be received 106 and used as input to a first machine learning module 114 that performs automated hotspot detection. The first machine learning module 114 automatically detects one or more hotspots 124 in the 3D functional image and generates a hotspot list 130 as output. A second machine learning module 116 may receive the hotspot list 130 as input along with the 3D functional image, and perform automated hotspot segmentation, 126 to generate a hotspot map 132. As previously described, the hotspot map 132, as well as the hotspot list 130, may be stored and/or provided for further processing 140.

In certain embodiments, a single machine learning module is trained to directly segment hotspots within images (e.g., 3D functional images; e.g., to generate a 3D hotspot map identifying volumes corresponding to detected hotspots), thereby combining the first two steps of detection and segmentation of hotspots. A second machine learning module may then be used to classify detected hotspots, for example based on the segmented hotspots determined previously. In certain embodiments, a single machine learning module may be trained to accomplish all three tasks - detection, segmentation, and classification - in a single step.

### III. Lesion Index Values

In certain embodiments, lesion index values are calculated for detected hotspots to provide a measure of, for example, relative uptake within and/or size of the corresponding physical lesion. In certain embodiments, lesion index values are computed for a particular hotspot based (i) on a measure of intensity for the hotspot and (ii) reference values corresponding to measures of intensity within one or more reference volumes, each corresponding to a particular reference tissue region. For example, in certain embodiments, reference values include an aorta reference value that measures intensity within an aorta volume corresponding to a portion of an aorta (also referred to as a blood pool reference) and a liver reference value that measures intensity within a liver volume corresponding to a liver of the subject. In certain embodiments, intensities of voxels of a nuclear medicine image, for example a PET image, represent standard uptake values (SUVs) (e.g., having been calibrated for injected radiopharmaceutical dose and/or patient weight), and measures of hotspot intensity and/or measures of reference values are SUV values. Use of such reference values in computing lesion index values is described in further detail, for example, in PCT/EP2020/050132, filed Jan. 6, 2020, the content of which is hereby incorporated by reference in its entirety.

In certain embodiments, a segmentation mask is used to identify a particular reference volume in, for example, a PET image. For a particular reference volume, a segmentation mask identifying the reference volume may be obtained via segmentation of an anatomical, e.g., CT, image. For example, in certain embodiments (e.g., as described in PCT/EP2020/050132), segmentation of a 3D anatomical image may be performed to produce a segmentation map, comprising a plurality of segmentation masks, each identifying a particular tissue region of interest. One or more segmentation masks of a segmentation map generated in this manner may, accordingly, be used to identify one or more reference volumes.

In certain embodiments, to identify voxels of the reference volume to be used for computation of the corresponding reference value, the mask may be eroded a fixed distance (e.g., at least one voxel), to create a reference organ mask that identifies a reference volume corresponding to a physical region entirely within the reference tissue region. For example, erosion distances of 3 mm and 9 mm may be used for aorta and liver reference volumes, respectively. Other erosion distances may also be used. Additional mask refinement may also be performed (e.g., to select a specific, desired, set of voxels for use in computing the reference value), for example as described below with respect to the liver reference volume.

Various measures of intensity within reference volumes may be used. For example, in certain embodiments, a robust average of voxel intensities inside the reference volume (e.g., as defined by the reference volume segmentation mask, following erosion) may be determined as a mean of values in an interquartile range of voxel intensities (IQRₘₑₐₙ). Other measures, such as a peak, a maximum, a median, etc. may also be determined. In certain embodiments, an aorta reference value is determined as a robust average of SUVs from voxels inside an aorta mask. The robust average is computed as the mean of the values in the interquartile range, IQRₘₑₐₙ.

In certain embodiments, a subset of voxels within a reference volume is selected in order to avoid impact from reference tissue regions that may have abnormally low radiopharmaceutical uptake. Although the automated segmentation techniques described and referenced herein can provide an accurate outline (e.g., identification) of regions of images corresponding to specific tissue regions, there are often areas of abnormally low uptake in the liver which should be excluded from the reference value calculation. For example, a liver reference value (e.g., a liver SUV value) may be computed so as to avoid impact from regions in the liver with very low tracer (radiopharmaceutical) activity, that might appear e.g., due to tumors without tracer uptake. In certain embodiments, to account for effects of abnormally low uptake in reference tissue regions the reference value calculation for the liver analyzes a histogram of intensities of voxels corresponding to the liver (e.g., voxels within an identified liver reference volume) and removes (e.g., excludes) intensities if they form a second histogram peak of lower intensities, thereby only including intensities associated with a higher intensity value peak.

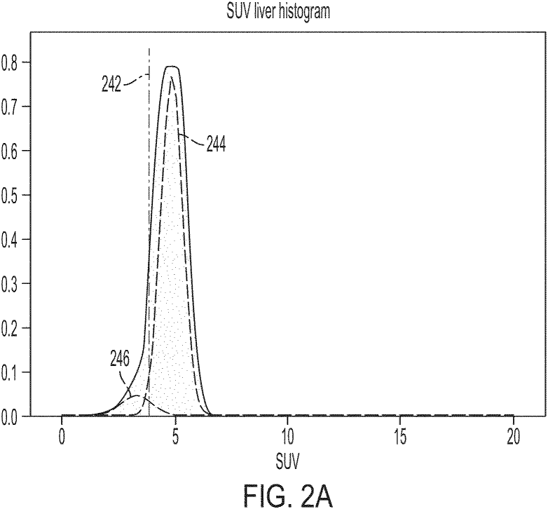

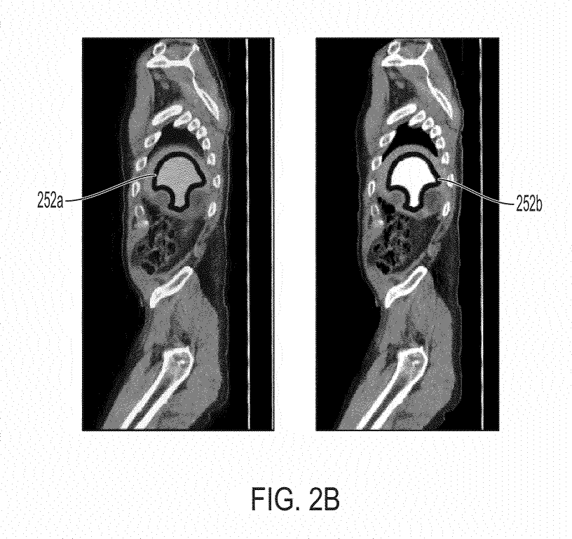

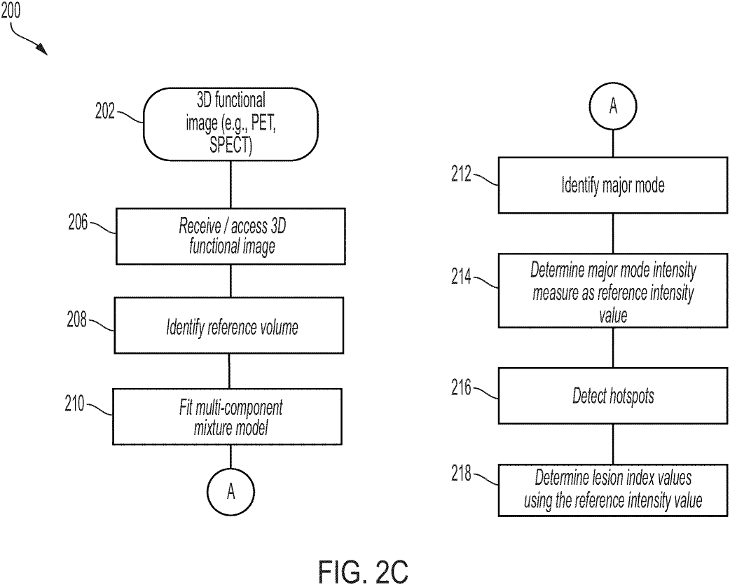

For example, for the liver, the reference SUV may be computed as a mean SUV of a major component (also referred to as “mode”, e.g., as in a “major mode”) in a two-component Gaussian Mixture Model fitted to a histogram of SUV’s of voxels within the liver reference volume (e.g., as identified by a liver segmentation mask, e.g., following the above-described erosion procedure). In certain embodiments, if a minor component has a larger mean SUV than the major component, and the minor component has at least 0.33 of the weight, an error is thrown and no reference value for the liver is determined. In certain embodiments, if the minor component has a larger mean than the major peak, the liver reference mask is kept as it is. Otherwise a separation SUV threshold is computed. In certain embodiments, the separation threshold defined such that a probability to belong to the major component for a SUV that is at the threshold or is larger is the same as the probability to belong to the minor component for a SUV that is at the separation threshold or is smaller. The reference liver mask is then refined by removing voxels with SUV smaller than the separation threshold. A liver reference value may then be determined as a measure of intensity (e.g., SUV) values of voxels identified by the liver reference mask, for example as described herein with respect to the aorta reference. FIG. 2A illustrates an example liver reference computation, showing a histogram of liver SUV values with Gaussian mixture components shown in red (major component 244 and minor component 246) and the separation threshold marked in green 242.

FIG. 2B shows the resulting portion of the liver volume used to calculate the liver reference value, with voxels corresponding to the lower value peak excluded from the reference value calculation. An outline 252a and 252b of a refined liver volume mask, with voxels corresponding to the lower value peak (e.g., having intensities below separation threshold 242) excluded, is shown on each image in FIG. 2B. As shown in the figure, lower intensity areas towards the bottom of the liver have been excluded, as well as regions close to the liver edge.

FIG. 2C shows an example process 200 where a multi-component mixture model is used to avoid impact from regions with low tracer uptake, as described herein with respect to liver reference volume computation. The process shown in FIG. 2C and described herein with regard to the liver may also be applied, similarly, to computation of intensity measures of other organs and tissue regions of interest as well, such as an aorta (e.g., aorta portion, such as the thoracic aorta portion or abdominal aorta portion), a parotid gland, a gluteal muscle. As shown, in FIG. 2C and described herein, in a first step, a 3D functional image 202 is received, and a reference volume corresponding to a specific reference tissue region (e.g., liver, aorta, parotid gland) is identified therein 208. A multi-component mixture model 210 is then fit to a distribution intensities (e.g., a histogram of intensities) of (e.g., within) the reference volume, and a major mode of the mixture model is identified 212. A measure of intensities associated with the major mode (e.g., and excluding contributions from intensities associated with other, minor, modes) is determined 214 and used as the reference intensity value for the identified reference volume. In certain embodiments, as described herein, the measure of intensities associated with the major mode is determined by identifying a separation threshold, such that intensities above the separation threshold are determined to be associated with the major mode, and intensities below it are determined to be associated with the minor mode. Voxels having intensities lying above the separation threshold are used to determine the reference intensity value, while voxels having intensities below the separation threshold are excluded from the reference intensity value calculation.

In certain embodiments, hotspots are detected 216 and the reference intensity value determined in this manner can be used to determine lesion index values for the detected hotspots 218, for example via approaches such as those described in PCT/US2019/012486, filed Jan. 7, 2019 and PCT/EP2020/050132, filed Jan. 6, 2020, the content of each of which is hereby incorporated by reference in its entirety.

### IV. Suppression of Intensity Bleed Associated With Normal Uptake in High-Uptake Organs

In certain embodiments, intensities of voxels of a functional image are adjusted in order to suppress / correct for intensity bleed associated with certain organs in which high-uptake occurs under normal circumstances. This approach may be used, for example, for organs such as a kidney, a liver, and a urinary bladder. In certain embodiments, correcting for intensity bleed associated with multiple organs is performed one organ at a time, in a stepwise fashion. For example, in certain embodiments, first kidney uptake is suppressed, then liver uptake, then urinary bladder uptake. Accordingly, the input to liver suppression is an image where kidney uptake has been corrected for (e.g., and input to bladder suppression is an image wherein kidney and liver uptake have been corrected for).

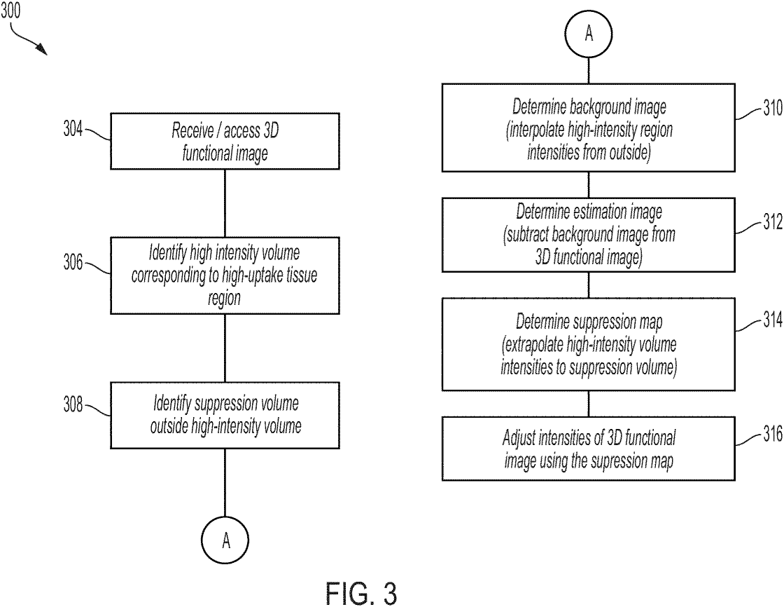

FIG. 3 shows an example process 300 for correcting intensity blead from a high-uptake tissue region. As shown in FIG. 3, a 3D functional image is received 304 and a high intensity volume corresponding to the high-uptake tissue region is identified 306. In another step, a suppression volume outside the high-intensity volume is identified 308. In certain embodiments, as described herein, the suppression volume may be determined as a volume enclosing regions outside of, but within a pre-determined distance from, the high-intensity volume. In another step, a background image is determined 310, for example by assigning voxels within the high-intensity volume intensities determined based on intensities outside the high-intensity volume (e.g., within the suppression volume), e.g., via interpolation (e.g., using convolution). In another step, an estimation image is determined 312 by subtracting the background image from the 3D functional image (e.g., via a voxel-by-voxel intensity subtraction). In another step, a suppression map is determined 314. As described herein, in certain embodiments, the suppression map is determined using the estimation image, by extrapolating intensity values of voxels within the high-intensity volume to locations outside the high intensity volume. In certain embodiments, intensities are only extrapolated to locations within the suppression volume, and intensities of voxels outside the suppression volume are set to 0. The suppression map is then used to adjust intensities of the 3D functional image 316, for example by subtracting the suppression map from the 3D functional image (e.g., performing a voxel-by-voxel intensity subtraction).

An example approach for suppression / correction of intensity bleed from a particular organ (in certain embodiments, kidneys are treated together) for a PET/CT composite image is as follows:
1. The projected CT organ mask segmentation is adjusted to high-intensity regions of the PET image, in order to handle PET/CT misalignment. If the PET-adjusted organ mask is less than 10 pixels, no suppression is made for this organ.
2. A “background image” is computed, replacing all high uptake with interpolated background uptake within the decay distance from the PET-adjusted organ mask. This is done using convolution with Gaussian kernels.
3. Intensities that should be accounted for when estimating suppression are computed as the difference between the input PET and the background image. This “estimation image” has high intensities inside the given organ and zero intensity at locations farther than the decay distance from the given organ.
4. A suppression map is estimated from the estimation image using an exponential model. The suppression map is only non-zero in the region within the decay distance of the PET-adjusted organ segmentation.
5. The suppression map is subtracted from the original PET image.

As described above, these five steps may be repeated, for each of a set of multiple organs, in a sequential fashion.

### V. Anatomical Labeling of Detected Lesions

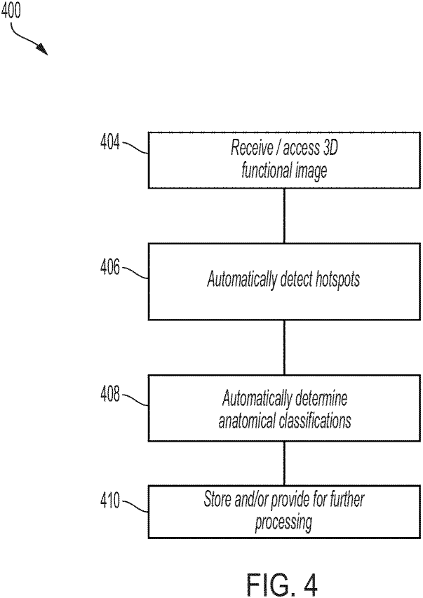

In certain embodiments, detected hotspots are (e.g., automatically) assigned anatomical labels that identify particular anatomical regions and/or groups of regions in which the lesions that they represent are determined to be located. For example, as shown in the example process 400 of FIG. 4, a 3D functional image may be received 404 an used to automatically detect hotspots 406, for example via any of the approaches described herein. Once hotspots are detected, anatomical classifications for each hotspot can be automatically determined 408 and each hotspot labeled with the determined anatomical classification. Automated anatomical labeling may, for example, be performed using automatically determined locations of detected hotspots along with anatomical information provided by, for example, a 3D segmentation map identifying image regions corresponding to particular tissue regions and/or an anatomical image. The hotspots and anatomical labeling of each may be stored and/or provided for further processing 410.

For example, detected hotspots may be automatically classified into one of five classes as follows:
T (prostate tumor)
N (pelvic lymph node)
Ma (non-pelvic lymph)
Mb (bone metastasis)
Mc (soft tissue metastasis not situated in prostate or lymphe node)

Table 1, below, lists tissue regions associated with each of the five classes. Hotspots corresponding to locations within any of the tissue regions associated with a particular class may, accordingly, be automatically assigned to that class.

TABLE 1

List of Tissue Regions Corresponding to Five Classes in a Lesion Anatomical Labeling Approach

| | | | | |
|---|---|---|---|---|
| Bone Mb | Lymph nodes Ma | Pelvic lymph nodes N | Prostate T | Soft tissue Mc |
| Skull | Cervical | Template right | Prostate | Brain |
| Thorax | Supraclavicular | Template left | | Neck |
| Vertebrae lumbar | Axillary | Presacral | | Lung |
| Vertebrae thoracic | Mediastinal | Other, pelvic | | Esophageal |
| Pelvis | Hilar | | | Liver |
| Extremities | Mesenteric | | | Gallbladder |
| | Elbow | | | Spleen |
| | Popliteal | | | Pancreas |
| | Peri-/para-aortic | | | Adrenal |
| | Other, non-pelvic | | | Kidney |
| | | | | Bladder |
| | | | | Skin |
| | | | | Muscle |
| | | | | Other |

### VI. Graphical User Interface and Quality Control and Reporting

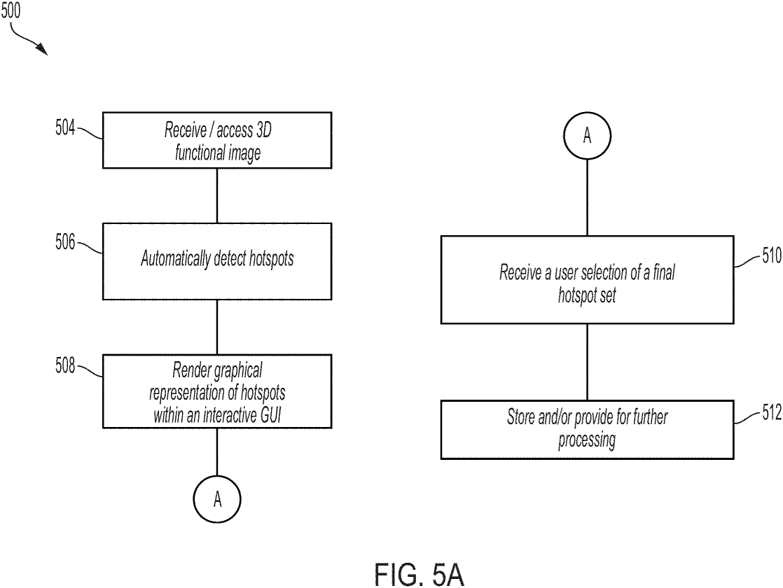

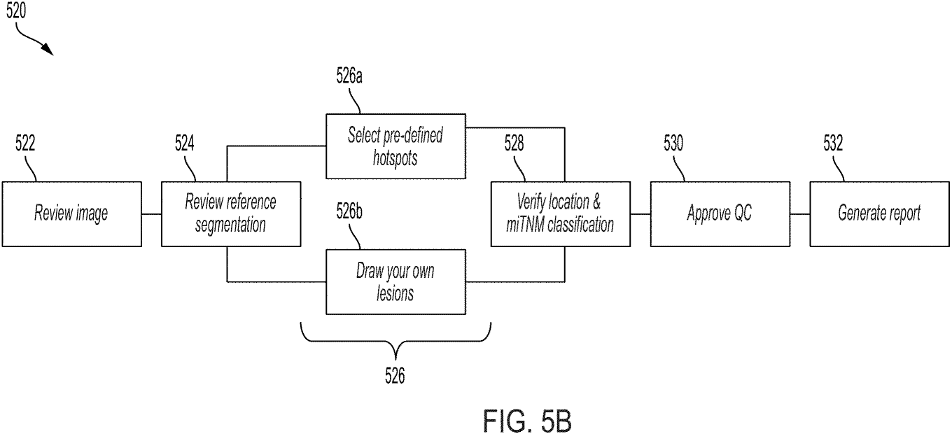

In certain embodiments, detected hotspots and associated information, such as computed lesion index values and anatomical labeling are displayed with an interactive graphical user interface (GUI) so as to allow for review by a medical professional, such as a physician, radiologist, technician, etc. Medical professionals may thus use the GUI to review and confirm accuracy of detected hotspots, as well as corresponding index values and/or anatomical labeling. In certain embodiments, the GUI may also allow users to identify, and segment (e.g., manually) additional hotspots within medical images, thereby allowing a medical professional to identify additional potential lesions that he/she believes the automated detection process may have missed. Once identified, lesion index values and/or anatomical labeling may also be determined for these manually identified and segmented lesions. For example, as indicated in FIG. 5B, the user may review locations determined for each hotspot, as well as anatomical labeling, such as a (e.g., automatically determined) miTNM classification. The miTNM classification scheme is described in further detail, for example, in Eiber et al., “Prostate Cancer Molecular Imaging Standardized Evaluation (PROMISE): Proposed miTNM Classification for the Interpretation of PSMA-Ligand PET/CT,” J. Nucl. Med., vol. 59, pg. 469-78 (2018), the content of which is hereby incorporated by reference in its entirety. Once a user is satisfied with the set of detected hotspots and information computed therefrom, they may confirm their approval and generate a final, signed report that can be reviewed and used to discuss outcomes and diagnosis with a patient, and assess prognosis and treatment options.

For example, as shown in FIG. 5A, in an example process 500 for interactive hotspot review and detection, a 3D functional image is received 504 and hotspots are automatically detected 506, for example using any of the automated detection approaches described herein. The set of automatically detected hotspots is represented and rendered graphically within an interactive GUI 508 for user review. The user may select at least a portion (e.g., up to all) of the automatically determined hotspots for inclusion in a final hotspot set 510, which may then be used for further calculations 512, e.g., to determine risk index values for the patient.

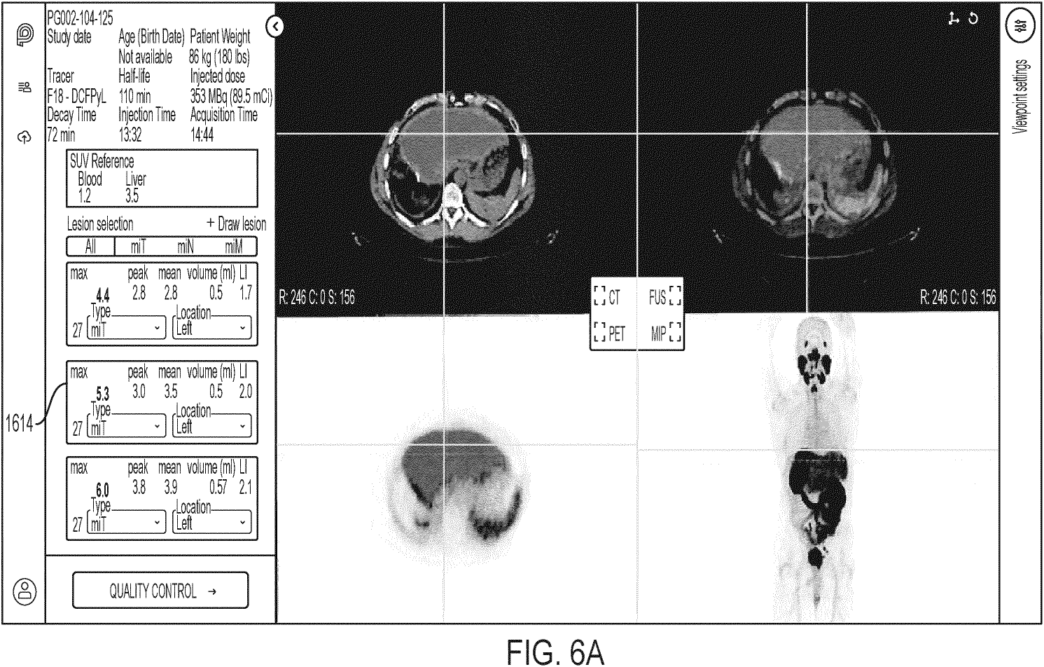

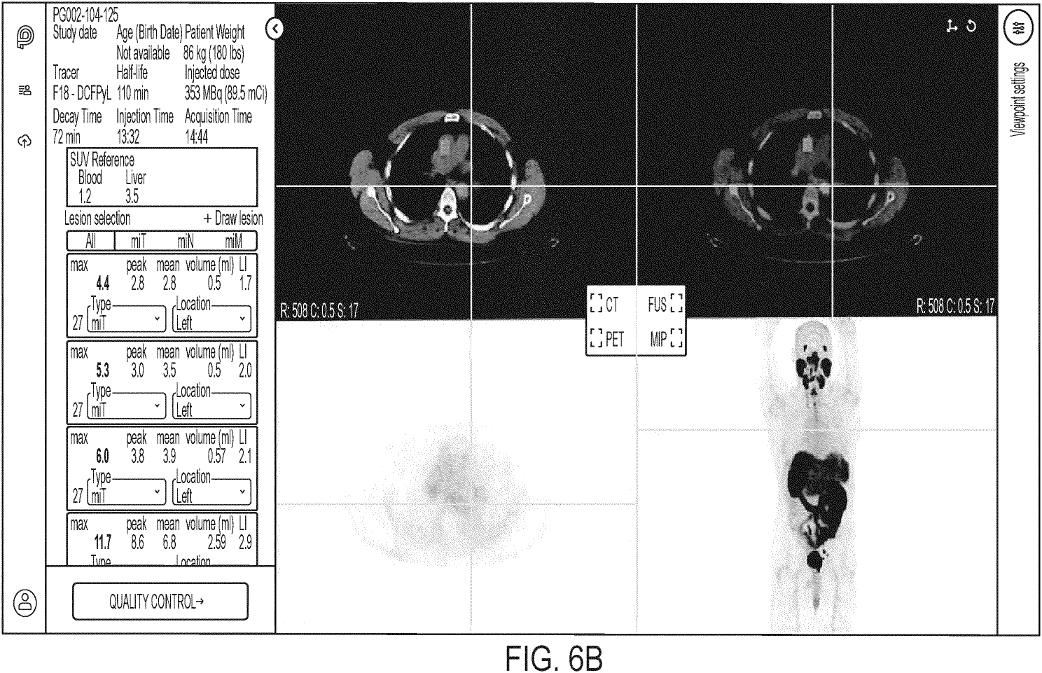

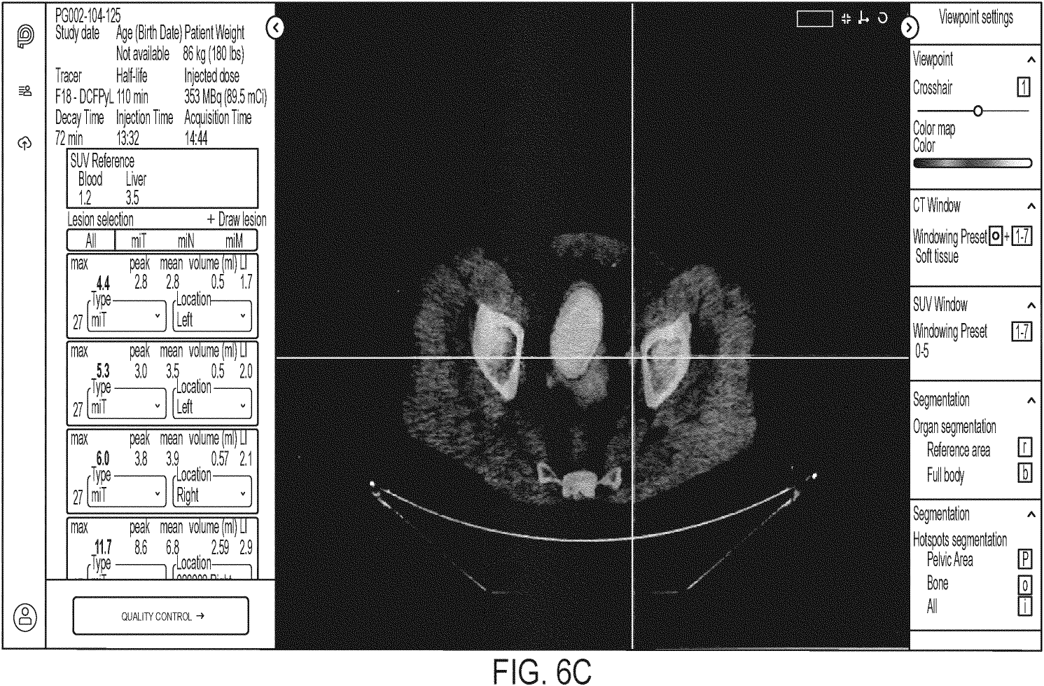

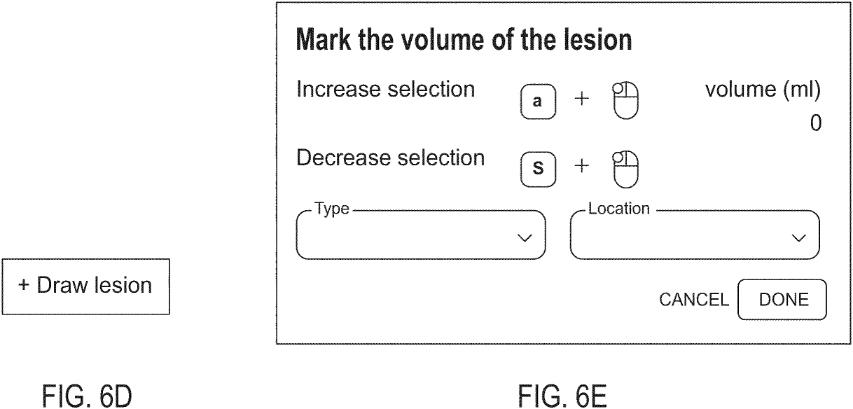

FIG. 5B shows an example workflow 520 for user review of detected lesions and lesion index values for quality control and reporting. The example workflow allows for user review of segmented lesions as well as liver and aorta segmentation used for calculation of lesion index values as described herein. For example, in a first step, a user reviews images (e.g., a CT image) for quality 522 and accuracy of automated segmentation used to obtain liver and blood pool (e.g., aorta) reference values 524. As shown in FIGS. 6A, and 6B the GUI allows a user evaluates images and overlaid segmentation to ensure that the automated segmentation of the liver (602, purple color in FIG. 6A) is within healthy liver tissue and that automated segmentation of blood pool (aorta portion 604, shown as salmon color in FIG. 6B is within the aorta and left ventricle.

In another step 526, a user validates automatically detected hotspots and/or identifies additional hotspots, e.g., to create a final set of hotspots corresponding to lesions, for inclusion in a generated report. As shown in FIG. 6C, a user may select an automatically identified hotspot by hovering over a graphical representation of the hotspot displayed within the GUI (e.g., as an overlay and/or marked region on a PET and/or CT image). To facilitate hotspot selection, the particular hotspot selected may be indicated to the user, via a color change (e.g., turning green). The user may then click on the hotspot to select it, which may be visually confirmed to the user via another color change. For example, as shown in FIG. 6C, upon selection the hotspot turns pink. Upon user selection, quantitatively determined values, such as a lesion index and/or anatomical labeling may be displayed to the user, allowing them to verify the automatically determined values 528.

In certain embodiments, the GUI allows a user to select hotspots from the set of (automatically) pre-identified hotspots to confirm they indeed represent lesions 526a and also to identify additional hotspots 562b corresponding to lesions, not having been automatically detected.

As shown in FIG. 6D and FIG. 6E, the user may use GUI tools to draw on slices of images (e.g., PET images and/or CT images; e.g., a PET image overlaid on a CT image) to mark regions corresponding to a new, manually identified lesion. Quantitative information, such as a lesion index and/or anatomical labeling may be determined for the manually identified lesion automatically, or may be manually entered by the user.

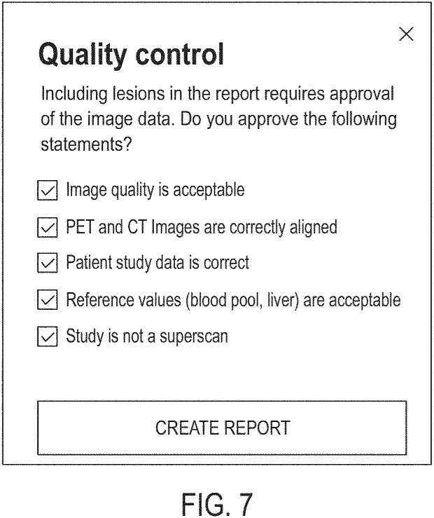

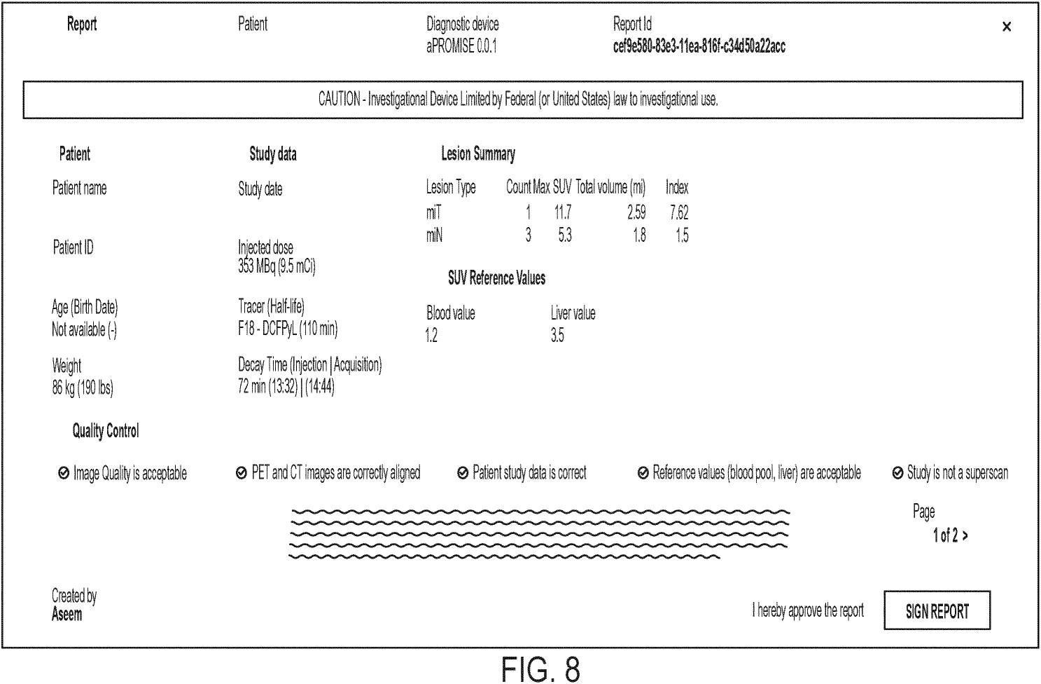

In another step, e.g., once the user has selected and/or manually identified all lesions, the GUI displays a quality control checklist for the user to review 530, as shown in FIG. 7. Once the user reviews and completes the checklist, they may click “Create Report” to sign and generate a final report 532. An example of a generated report is shown in FIG. 8.

### C. Example Machine Learning Network Architectures for Lesion Segmentation

### I. Machine Learning Module Input and Architecture

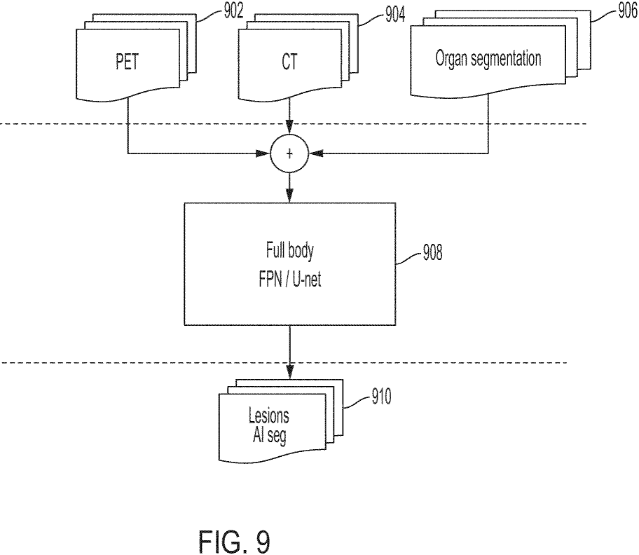

Turning to FIG. 9, which shows an example hotspot detection and segmentation process 900 in some embodiments, hotspot detection and/or segmentation is performed by a machine learning module 908 that receives, as input, a functional 902 and an anatomical 904 image, as well as a segmentation map 906 providing, for example, segmentation of various tissue regions such as soft-tissue and bone, as well as various organs as described herein.

Functional image 902 may be a PET image. intensities voxels of functional image 902, as described herein, may be scaled to represent SUV values. Other functional images as described herein may also be used, in certain embodiments. Anatomical image 904 may be a CT image. In certain embodiments, voxel intensities of CT image 904 are scaled to represent Hounsfield units. In certain embodiments, other anatomical images as described herein may be used.

In some embodiments, the machine learning module 908 implements a machine learning algorithm that uses a U-net architecture. In some embodiments, the machine learning module 908 implements a machine learning algorithm that uses a feature pyramid network (FPN) architecture. In some embodiments, various other machine learning architectures may be used to detect and/or segment lesions. In certain embodiments, machine learning modules as described herein perform semantic segmentation. In certain embodiments, machine learning modules as described herein perform instance segmentation, e.g., thereby differentiating one lesion from another.

In some embodiments, a three-dimensional segmentation map 906 received as input by a machine learning module identifies various volumes (e.g., via a plurality of 3D segmentation masks) in the received 3D anatomical and/or functional images as corresponding to particular tissue regions of interest, such as certain organs (e.g., prostate, liver, aorta, bladder, various other organs described herein, etc.) and/or bones. Additionally or alternatively, the machine learning module may receive a 3D segmentation map 906 that identifies groupings of tissue regions. For example, in some embodiments a 3D segmentation map that identifies soft-tissue regions, bone, and then background regions, may be used. In some embodiments, a 3D segmentation map may identify a group of high-uptake organs in which high levels of radiopharmaceutical uptake occur. A group of high-uptake organs may include, for example, a liver, spleen, kidneys and urinary bladder. In some embodiments, a 3D segmentation map identifies a group of high-uptake organs along with one or more other organs, such as an aorta (e.g., a low uptake soft tissue organ). Other groupings of tissue regions may also be used.

Functional image, anatomical image, and segmentation map input to machine learning module 908 may have various sizes and dimensionality. For example, in certain embodiments, each of functional image, anatomical image, and segmentation map are patches of three-dimensional images (e.g., represented by three dimensional matrices). In some embodiments, each of the patches has a same size - e.g., each input is a [32 × 32 × 32] or [64 × 64 × 64] patch of voxels.

Machine learning module 908 segments hotspots and generates a 3D hotspot map 910 identifying one or more hotspot volumes. For example, 3D hotspot map 910 may comprise one or more masks having a same size as one or more of functional image, anatomical image, or segmentation map input and identifying one or more hotspot volumes. In this manner, 3D hotspot map 910 may be used to identify volumes within functional image, anatomical image, or segmentation map corresponding to hotspots and, accordingly, physical lesions.

In some embodiments, machine learning module 908 segments hotspot volumes, differentiating between background (i.e., not hotspot) regions and hotspot volumes. For example, machine learning module 908 may be a binary classifier that classifies voxels as background or belonging to a single hotspot class. Accordingly, machine learning module 908 may generate, as output, a class agnostic (e.g., or ‘single-class’) 3D hotspot map that identifies hotspot volumes but does not differentiate between different anatomical locations and/or types of lesions - e.g., bone metastases, lymph nodules, local prostate - that particular hotspot volumes may represent. In some embodiments, machine learning module 908 segments hotspot volumes and also classifies hotspots according to a plurality of hotspot classes, each representing a particular anatomical location and/or type of lesion represented by a hotspot. In this manner, machine learning module 908 may, directly, generate a multi-class 3D hotspot map that identifies one or more hotspot volumes and labels each hotspot volume as belonging to a particular one of a plurality of hotspot classes. For example, detected hotspots may be classified as bone metastasis, lymph nodules, or prostate lesions. In some embodiments, other soft tissue classifications may be included.

This classification may be performed additionally or alternatively to classification of hotspots according to a likelihood that they represent a true lesion, as described herein, for example, in section B.ii.

### II. Lesion Classification Post-Processing and/or Output

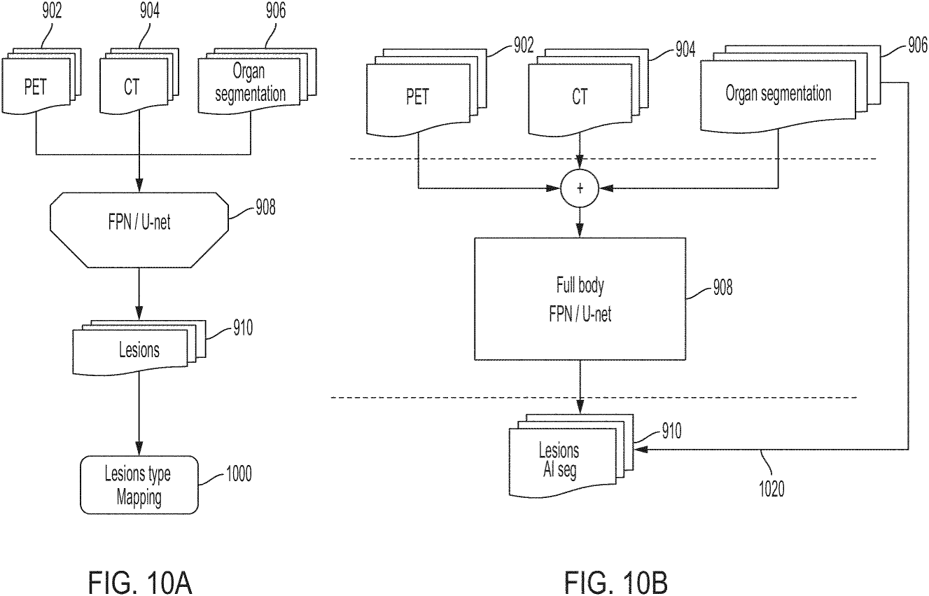

Turning to FIG. 10A and FIG. 10B, in some embodiments, following hotspot detection and/or segmentation (the terms “lesions” and “hotspots” are used interchangeably in FIGS. 9 - 12, with the understanding that physical lesions appear in, e.g., functional images, as hotspots) in images by one or more machine learning modules, post-processing 1000 is performed to label hotspots as belonging to a particular hotspot class. For example, detected hotspots may be classified as bone metastasis, lymph nodules, or prostate lesions. In some embodiments, the labeling scheme in Table 1 may be used. In some embodiments, such labeling may be performed by a machine learning module, which may be the same machine learning module used to perform segmentation and/or detection of hotspots, or may be a separate module that receives a listing of detected hotspots (e.g., identifying their locations) and/or a 3D hotspot map (e.g., delineating hotspot boundaries as determined via segmentation) as input, individually or along with other inputs, such as the 3D functional image, 3D anatomical image, and/or segmentation maps as described herein. As shown in FIG. 10B, in some embodiments, the segmentation map 906 used as input for the machine learning module 908 to perform lesion detection and/or segmentation may also be used to classify lesions, e.g., according to anatomical location. In some embodiments, other (e.g., different) segmentation maps may be used (e.g., not necessarily a same segmentation map that was fed into the machine learning module as input).

### III. Parallel Organ-Specific Lesion Detection Modules

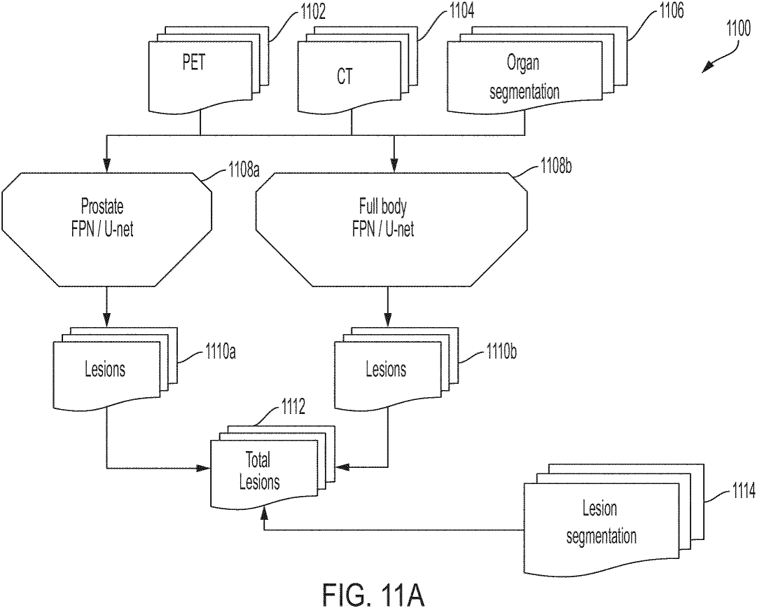

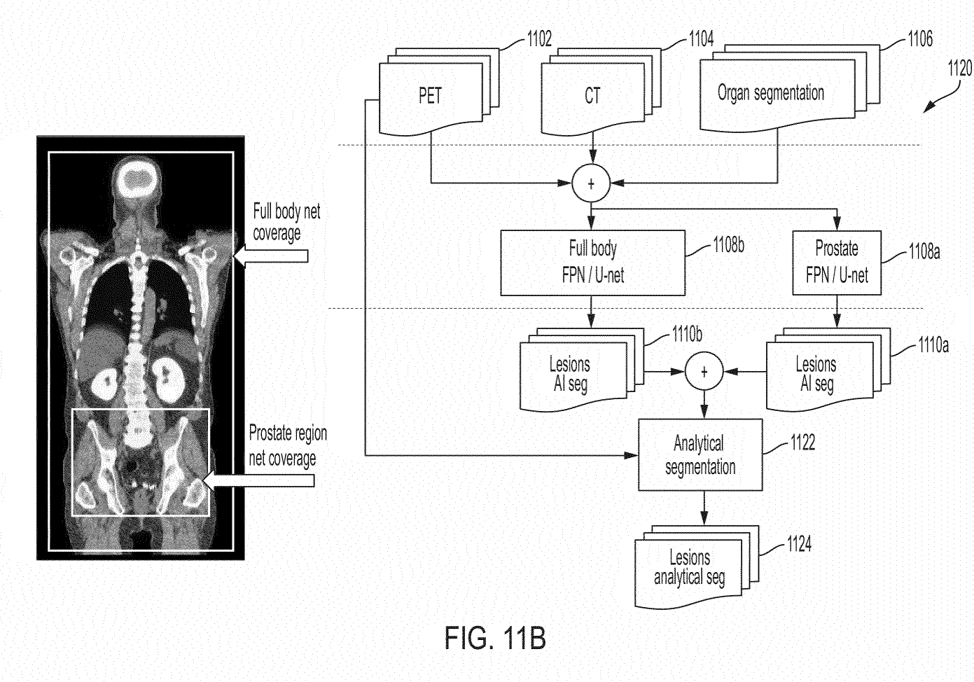

Turning to FIG. 11A and FIG. 11B, in some embodiments, the one or more machine learning modules comprise one or more organ specific modules that perform detection and/or segmentation of hotspots located in a corresponding organ. For example, as shown in example processes 1100 and 1150 of FIGS. 11A and 11B, respectively, a prostate module 1108a may be used to perform detection and/or segmentation in a prostate region. In some embodiments, the one or more organ specific modules are used in combination with a full body module 1108b that detects and or segments hotspots over an entire body of a subject. In some embodiments, results 1100a for the one or more organ specific modules are merged with results 11 10b from the full body module to form a final hotspot list and/or hotspot map 1112. In some embodiment, merging may include combining results (e.g., hotspot lists and/or 3D hotspot maps) 1110a and 1110b with other output, such as a 3D hotspot map 1114 created by segmenting hotspots using other methods, which may include use of other machine learning modules and/or techniques as well as other segmentation approaches. In some embodiments, an additional segmentation approach may be performed following detection and/or segmentation of hotspots by the one or more machine learning modules. This additional segmentation step may use, e.g., as input, hotspot segmentation and/or detection results obtained from the one or more machine learning modules. In certain embodiments, as shown in FIG. 11B, an analytical segmentation approach 1122 as described herein, e.g., in section C.iv. below, may be used along with organ specific lesion detection modules. Analytical segmentation 1122 uses results 1110b and 1110a from upstream machine learning modules 1108b and 1108a, along with PET image 1102 to segment hotspots using an analytical segmentation technique (e.g., which does not utilize machine learning) and creates an analytically segmented 3D hotspot map 1124.

### IV. Analytical Segmentation

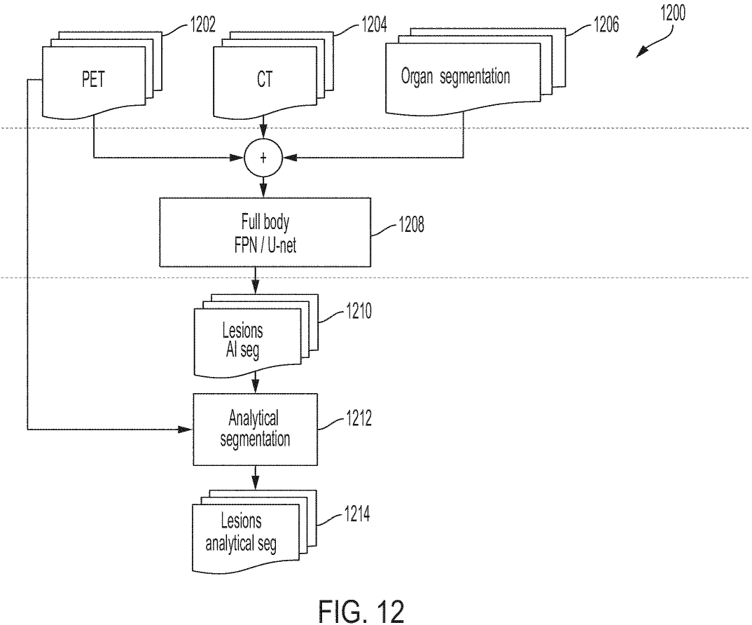

Turning to FIG. 12, in some embodiments, machine learning techniques may be used to perform hotspot detection and/or initial segmentation, and, e.g., as a subsequent step, an analytical model is used to perform a final segmentation for each hotspot.

As used herein, the terms “analytical model” and “analytical segmentation” refer to segmentation methods that are based on (e.g., use) predetermined rules and/or functions (e.g., mathematical functions). For example, in certain embodiments, an analytical segmentation method may segment a hotspot using one or more predetermined rules such as an ordered sequence of image processing steps, application of one or more mathematical functions to an image, conditional logic branches, and the like. Analytical segmentation methods may include, without limitation threshold-based methods (e.g., including an image thresholding step), level-set methods (e.g., a fast marching method), graph-cut methods (e.g., watershed segmentation), or active contour models. In certain embodiments, analytical segmentation approaches do not rely on a training step. In contrast, in certain embodiments, a machine learning model would segment a hotspot using a model that has been automatically trained to pre-segment hotspots using a set of training data (e.g., comprising examples of images and hotspots segmented, e.g., manually by a radiologist or other practitioner) and aims to mimic segmentation behavior in a training set.

Use of an analytical segmentation model to determine a final segmentation can be advantageous, e.g., since in certain cases analytical models may be more easily understood and debugged than machine learning approaches. In some embodiments, such analytical segmentation approaches may operate on a 3D functional image along with the lesion segmentation generated by the machine learning techniques.

For example, as shown in FIG. 12, in an example process 1200 for hotspot segmentation using an analytical model, machine learning module 1208 receives, as input, a PET image 1202, a CT image 1204, and a segmentation map 1206. Machine learning module 1208 performs segmentation to create a 3D hotspot map 1210 that identifies one or more hotspot volumes. Analytical segmentation model 1212 uses the machine learning module-generated 3D hotspot map 1210, along with PET image 1202 to perform segmentation and create 3D hotspot map 1214 that identifies analytically segmented hotspot volumes.

### V. Exemplary Hotspot Segmentation

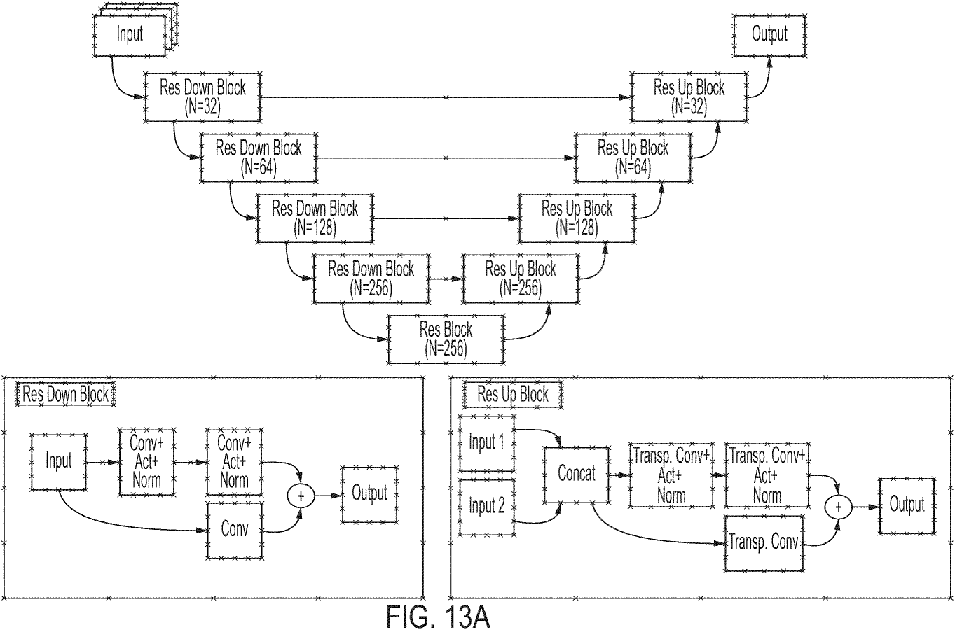

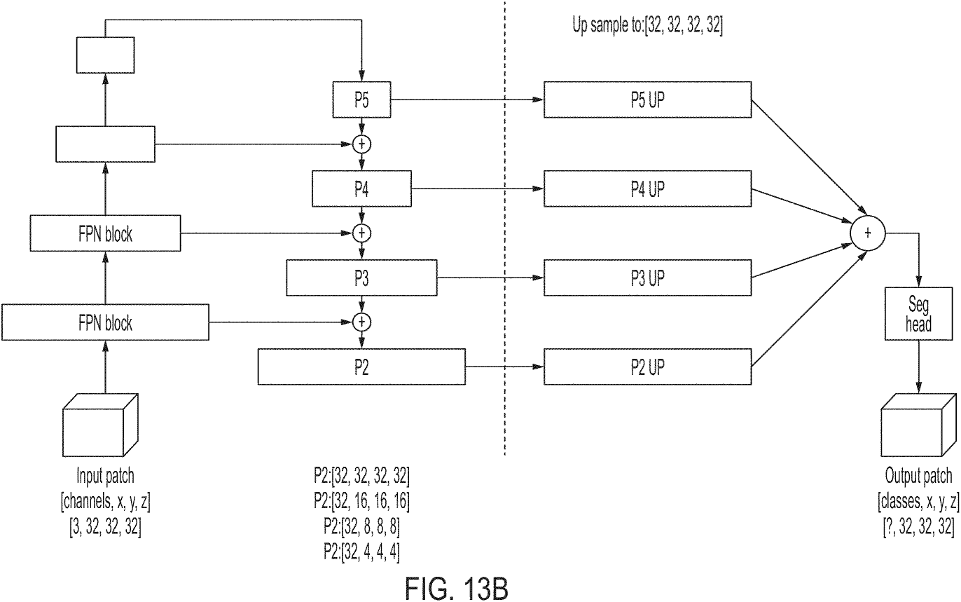

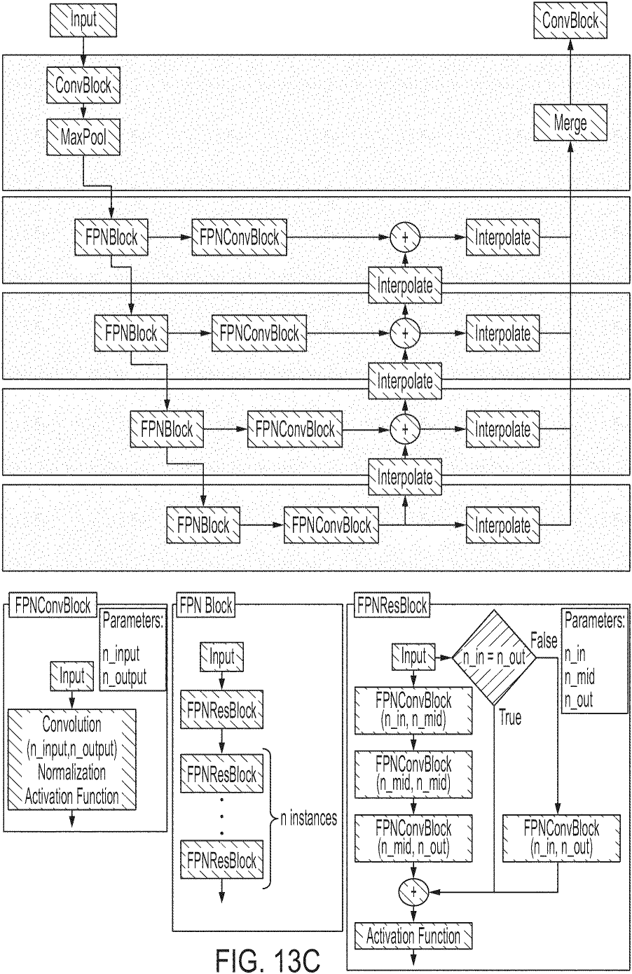

FIG. 13A and FIG. 13B show examples of machine learning module architectures for hotspot detection and/or segmentation. FIG. 13A shows an example U-net architecture (“N =” in the parentheticals of FIG. 13A identifies a number of filters in each layer) and FIG. 13B shows an example FPN architecture. FIG. 13C shows another example FPN architecture

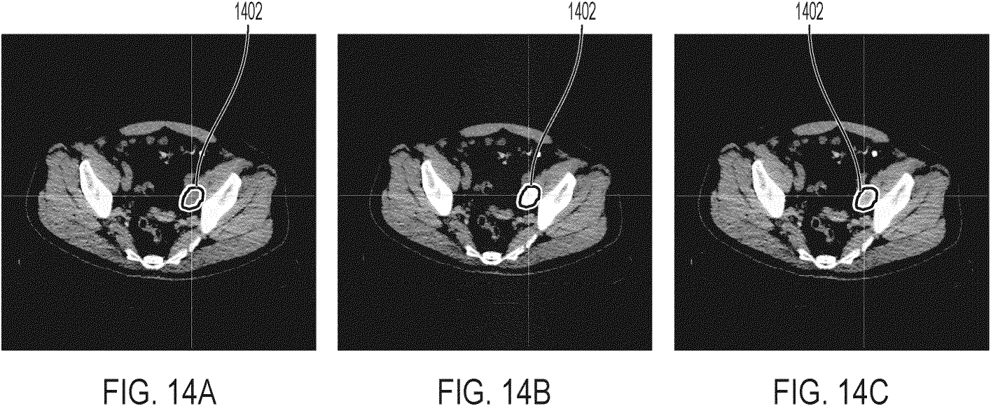

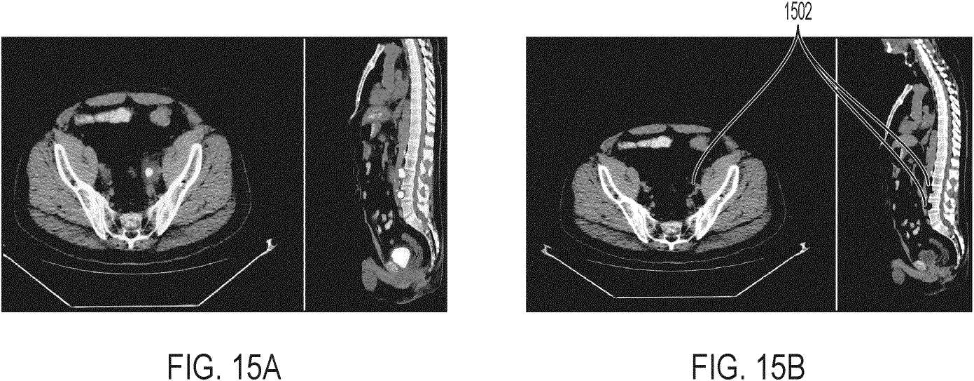

FIGS. 14A - C show example results for hotspot segmentation obtained using a machine learning module that implements a U-net architecture. Crosshairs and bright spots in the images indicate a hotspot 1402 (representing a potential lesion) that is segmented. FIGS. 15A and 15B show example hotspot segmentation results obtained using a machine learning module that implements a FPN. In particular, FIG. 15A shows an input PET image overlaid on a CT image. FIG. 15B shows an example hotspot map, determined using a machine learning module implementing a FPN, overlaid on the CT image. The overlaid hotspot map shows hotspot volumes 1502 in dark red, near the subject’s spine.

### D. Example Graphical User Interface

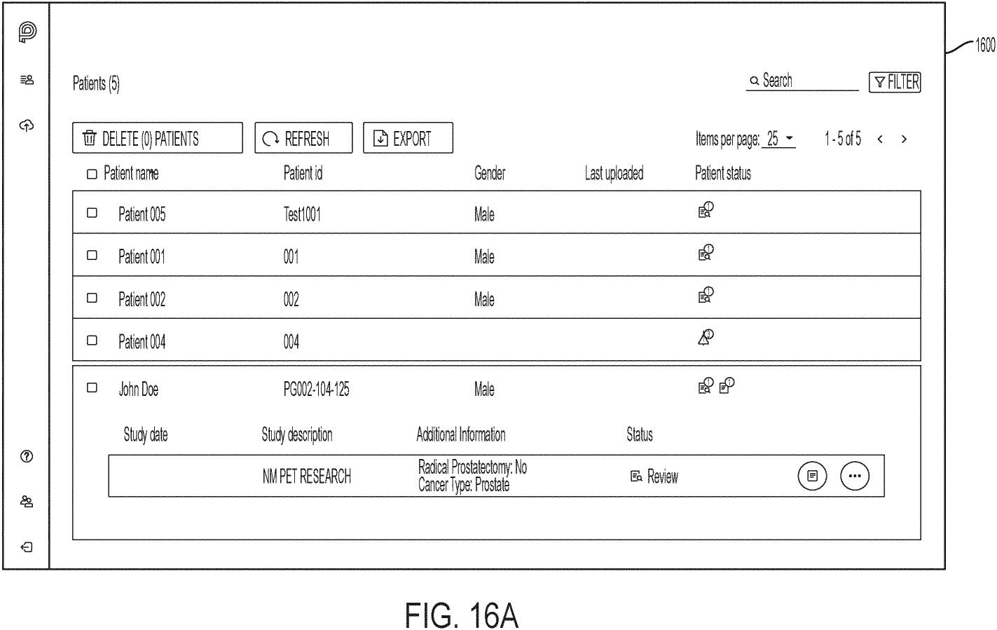

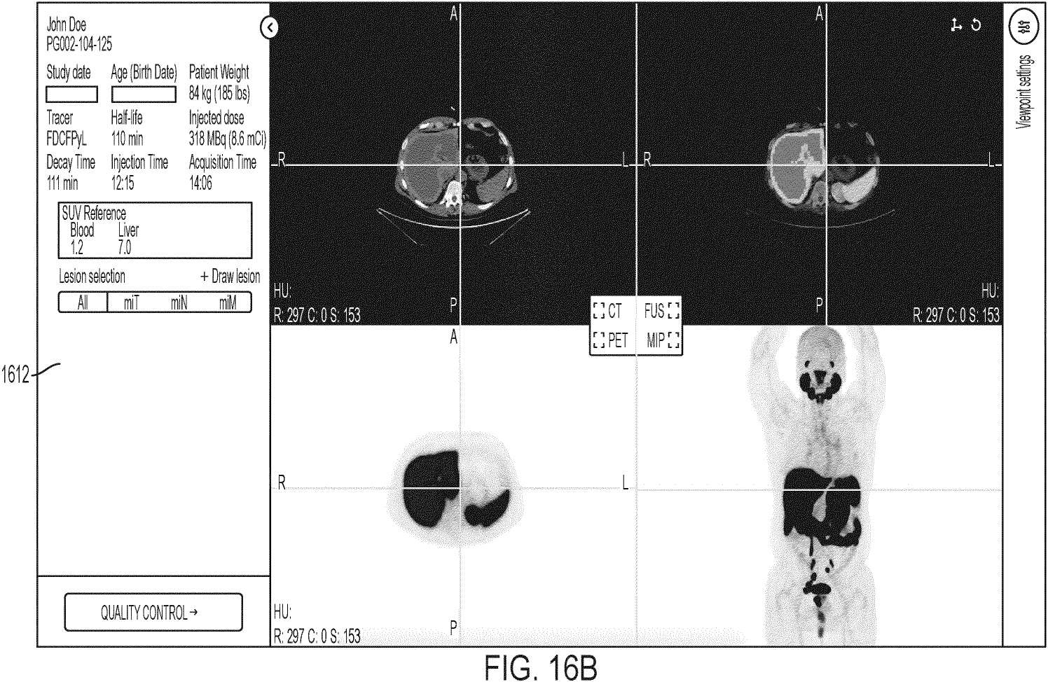

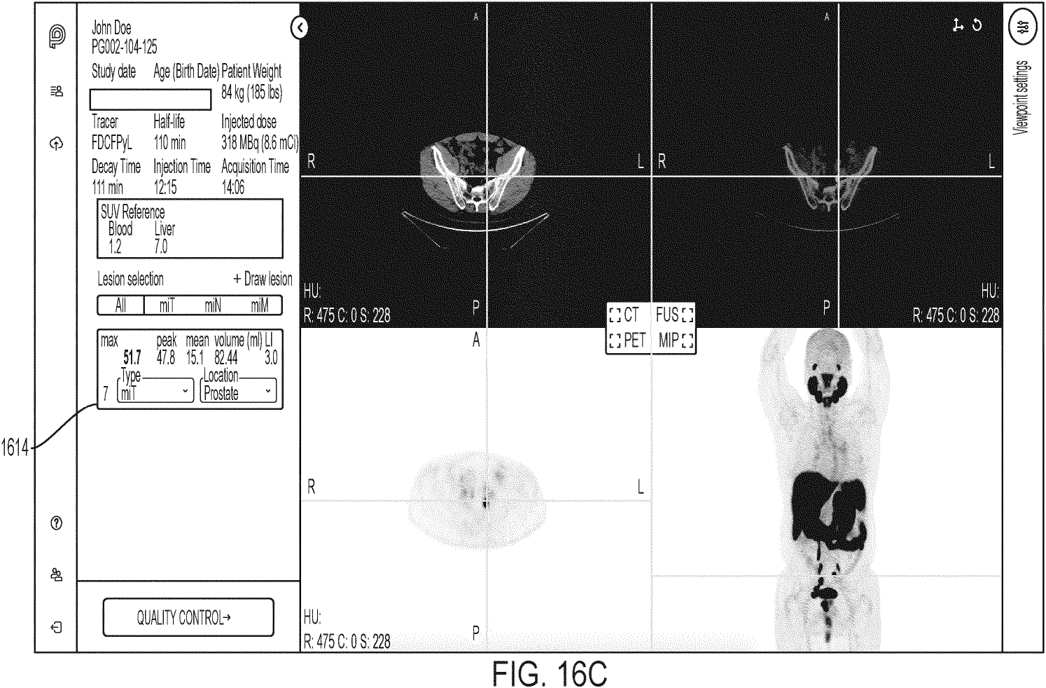

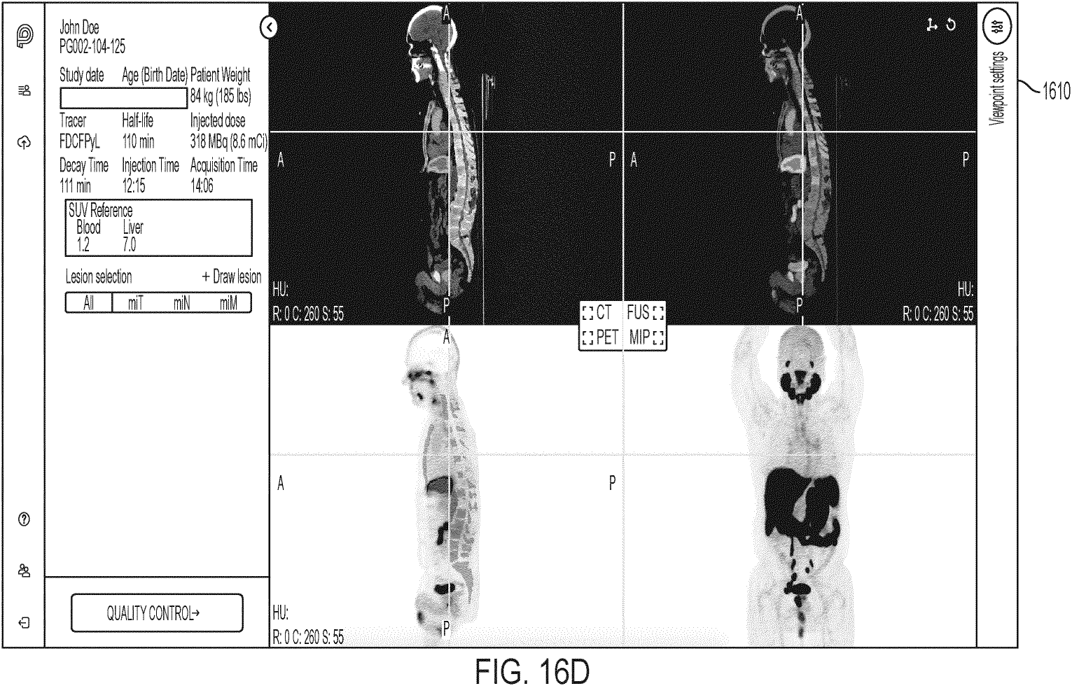

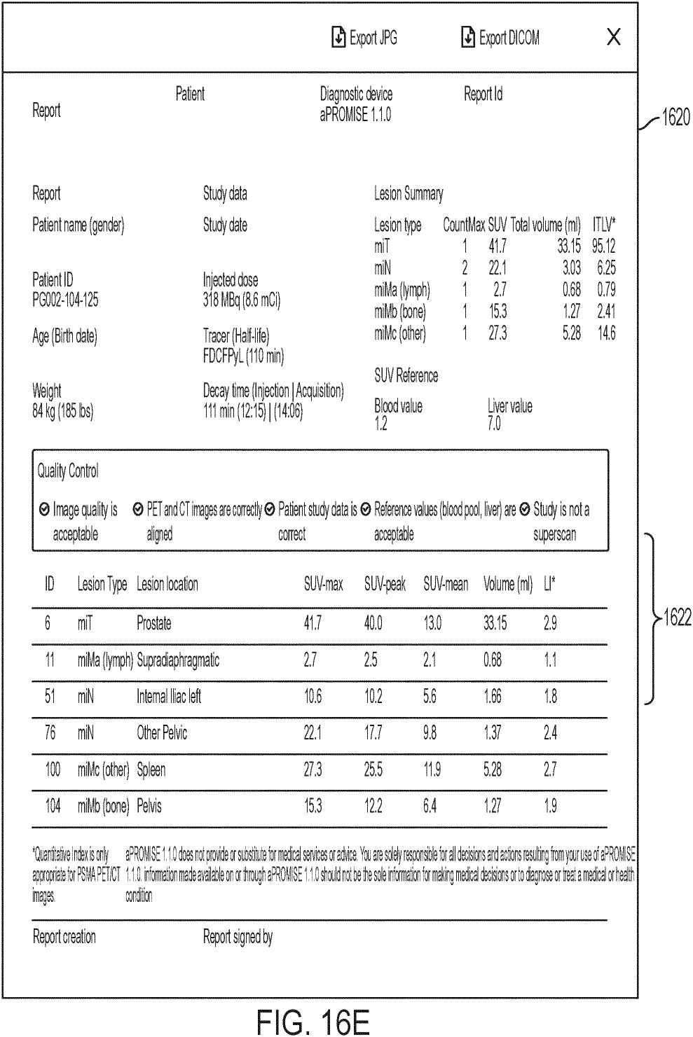

In certain embodiments, lesion detection, segmentation, classification and related technologies described herein may include a GUI that facilitates user interaction (e.g., with a software program implementing various approaches described herein) and/or review of results. For example, in certain embodiments, GUI portions and windows, allow, among other things, a user to upload and manage data to be analyzed, visualize images and results generated via approaches described herein, and generate a report summarizing findings. Screenshots of certain example GUI views are shown in FIGS. 16A - 16E.

For example, FIG. 16A shows an example GUI window providing for uploading and viewing of studies [e.g., image data collected during a same examination and/or scan (e.g., in accordance with a Digital Imaging and Communications in Medicine (DICOM) standard), such as a PET image and a CT image collected via a PET/CT scan) by a user. In certain embodiments, studies that are uploaded are automatically added to a patient list that lists identifiers of subjects/patients that have one or more PET/CT images uploaded. For each item in the patient list shown in FIGS. 16, a patient ID is shown along with available PET/CT studies for that patient, as well as corresponding reports.. In certain embodiments, a team concept allows for creation of a grouping of multiple users (e.g., a team) who work on, and are provided access to, a particular subset of uploaded data. In certain embodiments, a patient list may be associated with, and automatically shared with, a particular team, so as to provide each member of the team access to the patient list.

FIG. 16B shows an example GUI viewer 1610 that allows a user to view medical image data. In certain embodiments, the viewer is a multi-modal viewer, allowing a user to view multiple imaging modalities, as well as various formats and/or combinations thereof. For example, the viewer shown in FIG. 16B allows a user to view PET and/or CT images, as well as fusions (e.g., overlays) thereof. In certain embodiments, the viewer allows a user to view 3D medical image data in various formats. For example, the viewer may allow a user to select and view various 2D slices, along particular (e.g., selected) cross-sectional planes, of 3D images. In certain embodiments, the viewer allows a user to view a maximum intensity projection (MIP) of 3D image data. Other manners of visualizing 3D image data may also be provided. In this example, as shown in FIG. 16B, a control panel graphical widget 1612 is provided on the left-hand side of the viewer, and allows a user to view available study information, such as date, various patient data and imaging parameters, etc.

Turning to FIG. 16C, in certain embodiments a GUI viewer includes a lesion selection tool that allows a user to select lesion volumes that are volumes of interest (VOIs) of an image that the use identifies and selects as, e.g., likely to represent true underlying physical lesions. In certain embodiments, the lesion volumes are selected from a set of hotspots volumes that are automatically identified and segmented, for example via any of the approaches described herein. Selected lesion volumes may be saved for inclusion in a final set of identified lesion volumes that may be used for reporting and/or further quantitative analysis. In certain embodiments, for example, as shown in FIG. 16C, upon a user selection of a particular lesion volume, various features / quantitative metrics [e.g., a maximum intensity, a peak intensity, a mean intensity, a volume, a lesion index (LI), an anatomical classification (e.g., an miTNM class, a location, etc.), etc.] of the particular lesion are displayed 1614.

Turning to FIG. 16D, a GUI viewer may, additionally or alternatively, allow a user to view results of automated segmentation performed in accordance with various embodiments described herein. Segmentation may be performed via automated analysis of a CT image, as described herein, and may include identification and segmentation of 3D volumes representing a liver and/or aorta. Segmentation results may be overlaid on representations of medical image data, such as on a CT and/or PET image representation.

FIG. 16E shows an example report 1620 generated via analysis of medical image data as described herein. In this example, report 1620 summarizes results for the reviewed study and provides features and quantitative metrics characterizing selected (e.g., by the user) lesion volumes 1622. For example, as shown in FIG. 16E, the report includes, for each selected lesion volume, a lesion ID, a lesion type (e.g., a miTNM classification), a lesion location, a SUV-max value, a SUV-peak value, a SUV-mean value, a volume, and a lesion index value.

### E. Hotspot Segmentation and Classification Using Multiple Machine Learning Modules

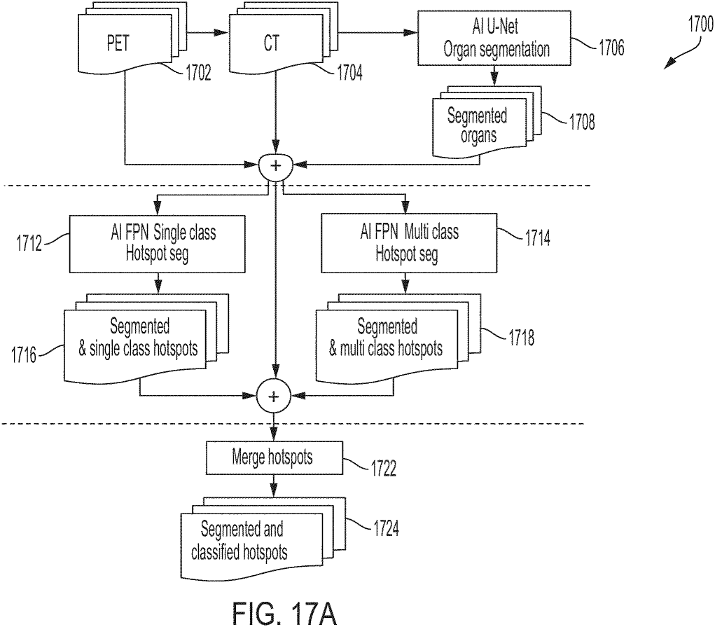

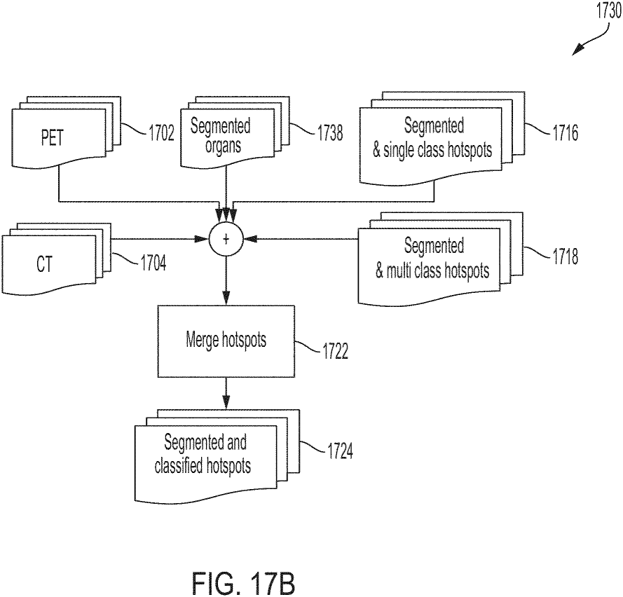

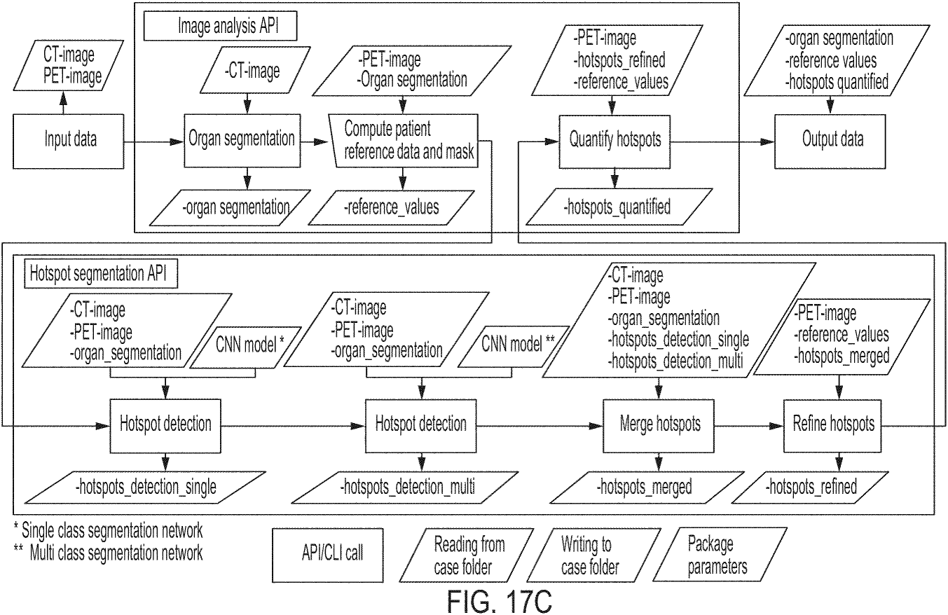

In certain embodiments, multiple machine learning modules are used in parallel to segment and classify hotspots. FIG. 17A is a block flow diagram of an example process 1700 for segmenting and classifying hotspots. Example process 1700 performs image segmentation on a 3D PET/CT image to segment hotspot volumes and classify each segmented hotspot volume according to a (automatically) determined anatomical location -in particular, as a lymph, bone, or prostate hotspot.

Example process 1700 receives as input, and operates on, a 3D PET image 1702 and a 3D CT image 1704. CT image 1704 is input to a first, organ segmentation, machine learning module 1706 that performs segmentation to identify 3D volumes in the CT image that represent particular tissue regions and/or organs of interest, or anatomical groupings of multiple (e.g., related) tissue regions and/or organs. Organ segmentation machine learning module 1706 is, accordingly, used to generate a 3D segmentation map 1708 that identifies, within the CT image, the particular tissue regions and/or organs of interest or anatomical groupings thereof. For example, in certain embodiments segmentation map 1708 identifies two volumes of interest corresponding two anatomical groupings of organs - one corresponding to an anatomical grouping of high uptake soft-tissue organs comprising a liver, spleen, kidneys, and a urinary bladder, and a second corresponding to an aorta (e.g., thoracic and abdominal part), which is a low uptake soft tissue organ. In certain embodiments, organ segmentation machine learning module 1706 generates an initial segmentation map as output that identifies various individual organs, including those that make up the anatomical groupings of segmentation map 1708, as well as, in certain embodiments, others, and segmentation map 1708 is created from the initial segmentation map (e.g., by assigning volumes corresponding to individual organs of an anatomical grouping a same label). Accordingly, in certain embodiments, 3D segmentation map 1708 uses three labels that identify and differentiate between (i) voxels belonging to the high uptake soft-tissue organs, (ii) low uptake soft tissue organ - i.e., the aorta, and (iii) other regions as background.

In example process 1700 shown in FIG. 17A organ segmentation machine learning module 1706 implements a U-net architecture. Other architectures (e.g., FPN’s) may be used. PET image 1702, CT image 1704 and 3D segmentation map 1708 are used as input to two parallel hotspot segmentation modules.

In certain embodiments, example process 1700 uses two machine learning modules in parallel to segment and classify hotspots in different manners, and then merges their results. For example, it was found that a machine learning module performed more accurate segmentation when it only identified a single class of hotpots - e.g., identifying image regions as hotspots or not - rather than the multiple - lymph, bone, prostate - desired hotspot classes. Accordingly, process 1700 utilizes a first, single class, hotspot segmentation module 1712 to perform accurate segmentation and a second, multi class, hotspot segmentation module 1714 to classify hotspots into the desired three categories.

In particular, a first, single class, hotspot segmentation module 1712 performs segmentation to generates a first, single class, 3D hotspot map 1716 that identifies 3D volumes representing hotspots, with other image regions identified as background. Accordingly, single class hotspot segmentation module 1712 performs a binary classification, labeling image voxels as belonging to one of two classes - background or a single hotspot class. A second, multi class, hotspot segmentation module 1714 segments hotspots and assigns segmented hotspot volumes one of a plurality of hotspot classification labels, as opposed to using a single hotspot class. In particular, multi class hotspot segmentation module 1714 classifies segmented hotspot volumes as lymph, bone, or prostate hotspots. Accordingly, multi class hotspot segmentation module generates a second, multi class, 3D hotspot map 1718 that identifies 3D volumes representing hotspots, and labels them as lymph, bone, or prostate, with other image regions identified as background. In process 1700, single class hotspot segmentation module and multi class hotspot segmentation module each implemented a FPN architecture. Other machine learning architectures (e.g., U-nets) may be used.

In certain embodiments, to generate a final 3D hotspot map of segmented and classified hotspots 1724, single class hotspot map 1716 and multi class hotspot map 1718 are merged 1722. In particular, each hotspot volume of single class hotspot map 1716 is compared with hotspot volumes of multi class hotspot map 1718 to identify matching hotspot volumes that represent the same physical location and, accordingly, a same (potential) physical lesion. Matching hotspot volumes may be identified, for example, based on various measures of spatial overlap (e.g., a percentage volume overlap), proximity (e.g., centers of gravity within a threshold distance), and the like. Hotspot volumes of single class hotspot map 1716 for which a matching hotspot volume from multi class hotspot map 1718 is/are identified are assigned a label - lymph, bone, or prostate - of the matching hotspot volume. In this manner, hotspots are accurately segmented via single class hotspot segmentation module 1712 and then labeled using the results of multi class hotspot segmentation module 1714.

Turning to FIG. 17B, in certain cases, for a particular hotspot volume of single class hotspot map 1716, no matching hotspot volume from multi class hotspot map 1718 is found. Such hotspot volumes are labeled based on a comparison with a 3D segmentation map 1738, which may be different from segmentation map 1708, that identifies 3D volumes corresponding to lymph and bone regions.

In certain embodiments, single class hotspot segmentation module 1712 may not segment hotspots in a prostate region such that single class hotspot map does not include any hotspots in a prostate regions. Hotspot volumes labeled as prostate hotspots from multi class hotspot map 1718 may be used for inclusion in merged hotspot map 1724. In certain embodiments, single class hotspot segmentation module 1712 may segment some hotspots in a prostate region, but additional hotspots (e.g., not identified in single class hotspot map 1716) may be segmented by and identified as prostate hotspots by multi class hotspot segmentation module 1714. These additional hotspot volumes, present in multi-class hotspot map 1718 may be included in merged hotspot map 1724.

Accordingly, in certain embodiments, information from a CT image 1704, a PET image 1702, a 3D organ segmentation map 1738, a single class hotspot map 1716 and a multi class hotspot map 1718 are used in a hotspot merging step 1722 to generate a merged 3D hotspot map of segmented and classified hotspot volumes 1724.

In one example merging approach, overlap (e.g., between two hotspot volumes) is determined when any two voxels of from a hotspot volume of multi class hotspot map and single class hotspot map that correspond to / represent a same physical location. If a particular hotspot volume of the single class hotspot map overlaps only one hotspot volume of the multi class hotspot map (e.g., only one matching hotspot volume from the multi class hotspot map identified), the particular hotspot volume of the single class hotspot map is labeled according to the class that the overlapping hotspot volume of the multi class hotspot map is identified as belonging to. If the particular hotspot volume overlaps two or more hotspot volumes of the multi-class hotspot map, each identified as belonging to a different hotspot class, then each voxel of the single class hotspot volume is assigned a same class as a closest voxel in an overlapped hotspot volume from the multi class hotspot map. If a particular hotspot volume of the single class hotspot map does not overlap any hotspot volume of the multi class hotspot map, the particular hotspot volume is assigned a hotspot class based on a comparison with an 3D segmentation map that identifies soft tissue regions (e.g., organs) and/or bone. For example, in some embodiments, the particular hotspot volume may be labeled as belonging to a bone class if any of the following statements is true:
(i) If more than 20% of the hotspot volume overlaps with a rib segmentation;
(ii) If the hotspot volume does not overlap with any label in the organ segmentation and the mean value of the CT in the hotspot mask is greater than 100 Hounsfield units;
(iii) If the position of the hotspot volume’s SUVₘₐₓ overlaps with a bone label in the organ segmentation; or
(iv) If more than 50% of the hotspot volume overlaps with a bone label in the organ segmentation.

In some embodiments, the particular hotspot volume may be identified as lymph if 50% or more of the hotspot volume does not overlap with a bone label in the organ segmentation.

In some embodiments, when all hotspot volumes of the single class hotspot map have been classified into lymph, bone or prostate, any remaining prostate hotspots from the multi class model are superimposed onto the single class hotpot map and included in the merged hotspot map.

FIG. 17C shows an example computer process 1750 for implementing a hotspot segmentation and classification approach in accordance with embodiments described with respect to FIGS. 17A and 17B.

### F. Analytical Segmentation via an Adaptive Thresholding Approach

In certain embodiments, for example as described herein in Section C.iv, image analysis techniques described herein utilize an analytical segmentation step to refine hotspot segmentations determined via machine learning modules as described herein. For example, in certain embodiments a 3D hotspot map generated by machine learning approaches as described herein is used as an initial input to an analytical segmentation model that refines and/or performs an entirely new segmentation.

In certain embodiments, an analytical segmentation model utilizes a thresholding algorithm, whereby hotspots are segmented by comparing intensities of voxels in an anatomical image (e.g., a CT image, an MR image) and/or functional image (e.g., a SPECT image, a PET image) (e.g., a composite anatomical and functional image, such as a PET/CT or SPECT/CT image) with one or more threshold values.

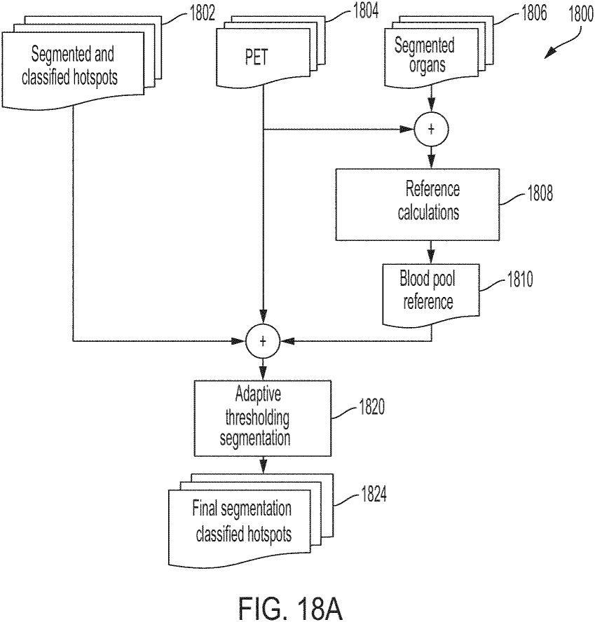

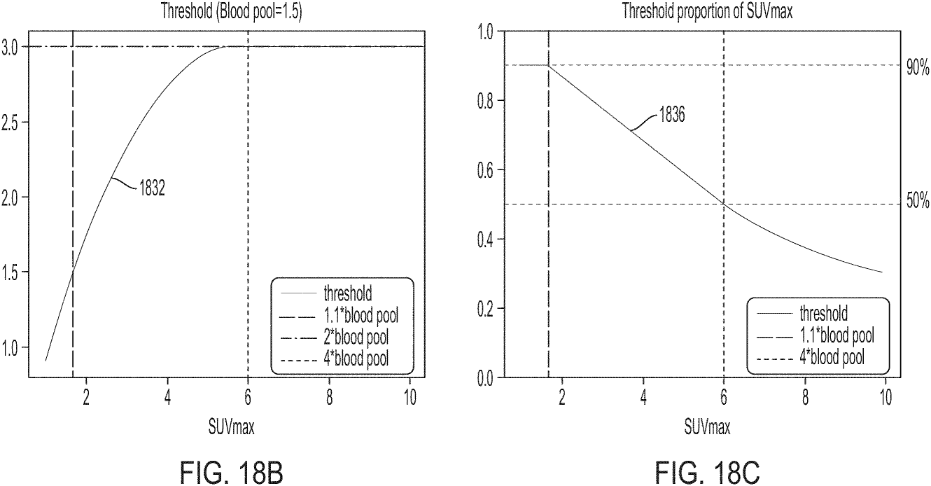

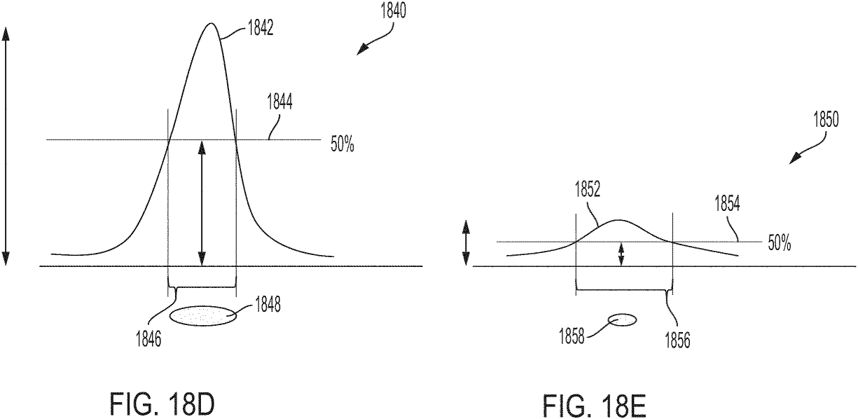

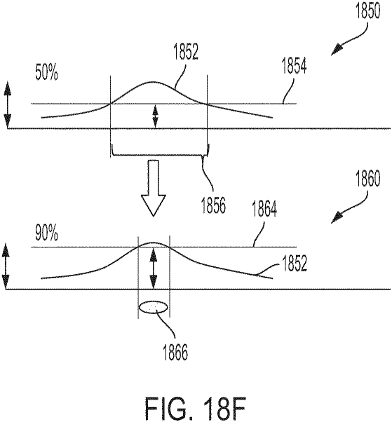

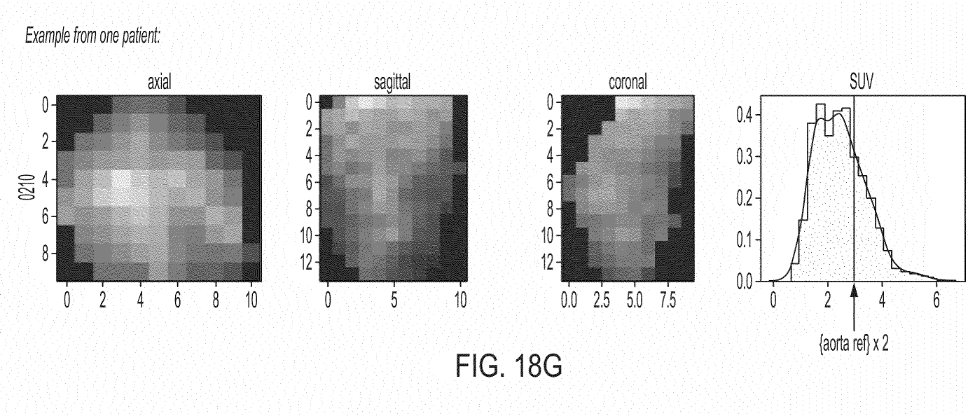

Turning to FIG. 18A, in certain embodiments, an adaptive thresholding approach, whereby for a particular hotspot, intensities within an initial hotspot volume determined for the particular hotspot, for example via machine learning approaches as described herein, are compared with one or more reference values to determine a threshold value for the particular hotspot. The threshold value for the particular hotspot is then used by an analytical segmentation model to segment the particular hotspot and determine a final hotspot volume.

FIG. 18A shows an example process 1800 for segmenting hotspots via an adaptive thresholding approach. Process 1800 utilizes an initial 3D hotspot map 1802 that identifies one or more 3D hotspot volumes, a PET image 1804, and a 3D organ segmentation map 1806. Initial 3D hotspot map 1802 may be determined automatically, via various machine learning approaches described herein and/or based on a user interaction with a GUI. A user may, for example, refine a set of automatically determined hotspot volumes by selecting a subset for inclusion in 3D hotspot map 1802. Additionally or alternatively, a user may determine 3D hotspot volumes manually, for example by drawing boundaries on an image with a GUI.

In certain embodiments, 3D organ segmentation map identifies one or more reference volumes that correspond to particular reference tissue regions, such as an aorta portion and/or a liver. As described herein, for example in Section B.iii, intensities of voxels within certain reference volumes may be used to compute associated reference values 1808, against which intensities of identified and segmented hotspots can be compared (e.g., acting as a ‘measuring stick’). For example, a liver volume may be used to compute a liver reference value and an aorta portion used to compute an aorta or blood pool reference value. In process 1800, intensities of an aorta portion are used to compute 1808 a blood pool reference value 1810. Blood pool reference value 1810 is used in combination with initial 3D hotspot map 1802 and PET image 1804 to determine threshold values for performing a threshold-based analytical segmentation of hotspots in initial 3D hotspot map 1802.

In particular, for a particular hotspot volume (which identifies a particular hotspot, representing a physical lesion) identified in initial 3D hotspot map 1802, intensities of PET image 1804 voxels located within the particular hotspot volume are used to determine an hotspot intensity for the particular hotspot. In certain embodiments, the hotspot intensity is a maximum of intensities of voxels located within the particular hotspot volume. For example, for PET image intensities representing SUVs, a maximum SUV (SUVₘₐₓ) within the particular hotspot volume is determined. Other measures, such as a peak value (e.g., SUVₚₑₐₖ), mean, median, interquartile mean (IQRₘₑₐₙ) may be used.

In certain embodiments, a hotspot-specific threshold for the particular hotspot is determined based on a comparison of the hotspot intensity with a blood pool reference value. In certain embodiments, a comparison between the hotspot intensity and the blood pool reference value is used to select one of a plurality of (e.g., predefined) threshold functions, and the selected threshold function used to compute the hotspot-specific threshold value for the particular hotspot. In certain embodiments, a threshold function computes a hotspot-specific threshold value as a function of the hotspot intensity (e.g., a maximum intensity) of the particular hotspot and/or the blood pool reference value. For example, a threshold function may compute the hotspot-specific threshold value as a product of (i) a scaling factor and (ii) the hotspot intensity (or other intensity measure) of the particular hotspot and/or the blood pool reference. In certain embodiments, the scaling factor is a constant. In certain embodiments, the scaling factor is an interpolated value, determined as a function of the intensity measure of the particular hotspot. In certain embodiments and/or for certain threshold functions, the scaling factor is a constant, used to determine a plateau level corresponding to a maximum threshold value, for example as described in further detail in Section G herein.

For example, pseudocode for an example approach that selects between (e.g., via conditional logic) and computing various threshold functions is shown below:
If 90% of SUVₘₐₓ ≤ [blood pool reference], then threshold= 90% of SUVmax.
Else, if 50% of SUVₘₐₓ ≥ 2 x [blood pool reference], then threshold = 2 x [blood pool reference].
Else, use linear interpolation to determine a percentage of SUVmax, with the interpolation starting at 90% at [[blood pool reference] / 0.9] and ending at 50% at [2 x [blood pool reference] / 0.5].
   If [interpolated percentage] x SUVmax is below 2 x [blood pool reference], then threshold = [interpolated percentage] x SUVmax.
   Else, threshold = 2 x [blood pool reference].

FIGS. 18B and 18C illustrate the particular example adaptive thresholding approach implemented by the pseudo code above. FIG. 18B plots variation in threshold value 1832 as a function of hotspot intensity -SUVₘₐₓ in the example - for a particular hotspot. FIG. 18C plots variation in hotspot-specific threshold value as proportion of SUVₘₐₓ for a particular hotspot, as a function of SUVₘₐₓ for the particular hotspot. Dashed lines in each graph indicate certain values relative to a blood pool reference (having a SUV of 1.5 in the example plots of FIGS. 18B and 18C), and also indicate 90% and 50% of SUVₘₐₓ in FIG. 18C.

Turning to FIGS. 18D-F, adaptive thresholding approaches as described herein address challenges and shortcomings associated with previous thresholding techniques that utilize fixed or relative thresholds. In particular, while threshold-based lesion segmentation based on a maximum Standard Uptake Value (SUVₘₐₓ) provides, in certain embodiments, a transparent and reproducible way to segment hotspot volumes for estimation of parameters such as uptake volume and SUVₘₑₐₙ, conventional fixed and relative thresholds do not work well under the full dynamic range of lesion SUVₘₐₓ. A fixed threshold approach uses a single, e.g., user defined, SUV value as a threshold for use in segmenting hotspots within an image. For example, a user might set a fixed threshold level at a value of 4.5. A relative threshold approach uses a particular, constant, fraction or percentage and segments hotspots using a local threshold for each hotspot, set at the particular fraction or percentage of the hotspot maximum SUV. For example, a user may set a relative threshold value at 40%, such that each hotspot is segmented using a threshold value calculated as 40% of the maximum hotspot SUV value. Both these approaches - conventional fixed and relative thresholds suffer from drawbacks. For example, it is difficult to define appropriate fixed thresholds that work well across patients. Conventional relative threshold approaches, are also problematic, since defining a threshold value as a fixed fraction of hotspot maximum or peak intensity results in hotspots with lower overall intensities being segmented using lower threshold values. As a result, segmenting low intensity hotspots, which may represent smaller lesions with relatively low uptake, using a low threshold value, may result in a larger identified hotspot volume than for a higher intensity hotspot that in fact represents a physically larger lesion.

For example, FIGS. 18D and 18E illustrate segmentation of two hotspots using a threshold value determined as 50% of a maximum hotspot intensity (e.g., 50% SUVₘₐₓ). Each figure plots intensity on the vertical as a function of position, showing a line cut through a hotspot. FIG. 18D shows a graph 1840 illustrating variation in intensity for a high intensity hotspot, representing a large physical lesion 1848. Hotspot intensity 1842 peaks about a center of a hotspot and hotspot threshold value 1844 is set a 50% of maximum of hotspot intensity 1842. Segmenting the hotspot using hotspot threshold value 1844 produces a segmented volume that approximately matches a size of the physical lesion, as shown, for example by comparing linear dimension 1846 with illustrated lesion 1848. FIG. 18E shows a graph 1850 illustrating variation in intensity for a low intensity hotspot, representing a small physical lesion 1858. Hotspot intensity 1852 also peaks about a center of a hotspot and hotspot threshold value 1854 is also set a 50% of maximum hotspot intensity 1852. However, since hotspot intensity 1852 peaks less sharply, and has a lower intensity peak, than hotspot intensity 1842 for the high intensity hotspot, setting a threshold value relative to a maximum of the hotspot intensity results in a much lower absolute threshold value. As a result, threshold based segmentation produces a hotspot volume that is larger in comparison with the that of the higher intensity hotspot, although the physical lesion represented is smaller, as shown, for example by comparing linear dimension 1856 with illustrated lesion 1858. Relative thresholds, may, accordingly, produce larger apparent hotspot volumes for smaller physical lesions. This is particularly problematic for assessment of treatment response, since lower-intensity lesions will have lower thresholds and, accordingly, a lesion responding to treatment may appear to increase in volume.

In certain embodiments, adaptive thresholding as described herein addresses these shortcomings by utilizing an adaptive threshold that is computed as percentage of hotspot intensity, the percentage (i) decreasing with increasing hotspot intensity (e.g., SUVₘₐₓ) and (ii) dependent on both hotspot intensity (e.g., SUVₘₐₓ) and overall physiological uptake (e.g., as measured by a reference value, such as a blood pool reference value). Accordingly, unlike a conventional relative thresholding approach, the particular fraction / percentage of hotspot intensity used in the adaptive thresholding approach described herein varies, and is itself a function of hotspot intensity and also, in certain embodiments, accounts for physiological uptake as well. For example, as shown in the illustrative plot 1860 of FIG. 18F, utilizing a variable, adaptive thresholding approach as described herein sets threshold value 1864 as a higher percentage - e.g., 90% as shown in FIG. 18F - of peak hotspot intensity 1852. As illustrated in FIG. 18F, doing so allows for threshold-based segmentation to identify a hotspot volume that more accurately reflects a true size of a lesion 1866 the hotspot represents.

In certain embodiments, thresholding is facilitated by first splitting heterogeneous lesions into homogeneous sub-components, and finally excluding uptake from nearby intensity peaks, using a watershed algorithm. As described herein, adaptive thresholding can be applied to manually pre-segmented lesions as well as automated detections by, for example deep neural networks implemented via machine learning modules as described herein, to improve reproducibility and robustness and to add explainability.

### G. Example Study Comparing Example Threshold Functions and Scaling Factors for PYL-PET/CT Imaging

This example describes a study performed to evaluate various parameters for use in an adaptive thresholding approach, as described herein, for example in Section F, and compare fixed and relative thresholds, using manually annotated lesions as a reference.

The study of this example used ¹⁸F-DCFPyL PET/CT scans of 242 patients, with hotspots corresponding to bone, lymph and prostate lesions manually segmented by an experienced nuclear medicine reader. In total 792 hotspot volumes were annotated, across 167 patients. Two studies were performed to assess thresholding algorithms. In a first study, manually annotated hotspots were refined with different thresholding algorithms, and it was estimated how well size order was preserved, i.e., to what extent smaller hotspot volumes remained smaller than initially larger hotspot volumes after refinement. In a second study, refinement by thresholding of suspicious hotspots automatically detected by a machine learning approach in accordance with various embodiments described herein, was performed and compared to the manual annotations.

PET image intensities in this example were scaled to represent standard uptake values (SUV), and are referred to in this section as uptake or uptake intensities. Different thresholding algorithms that were compared are as follows: a fixed threshold at SUV=2.5, a relative threshold of 50% of SUVₘₐₓ, and variants of adaptive thresholds. The adaptive thresholds were defined from a decreasing percentage of SUVₘₐₓ, with and without a maximal threshold level. A plateau level was set so as to be above normal uptake intensities in regions corresponding to healthy tissues. Two supporting investigations were performed to select an appropriate plateau level: one studying normal uptake intensities in the aorta, and one studying normal uptake intensities in the prostate. Among other things, thresholding approaches were evaluated based on their preservation of size order in comparison with annotations performed by a nuclear medicine reader. For example, if a nuclear medicine reader segmented hotspots manually, and the manually segmented hotspot volumes ordered according to size, preservation of size order refers to a degree to which hotspot volumes produced by segmenting the same hotspots using an automated thresholding approach (e.g., that does not include a user interaction) would be ordered according to their size in the same way. Two embodiments of an adaptive thresholding approach achieved best performance in terms of size order preservation, according to a weighted rank correlation measure. Both of these adaptive thresholding methods utilized thresholds that started at 90% of SUVₘₐₓ for low intensity lesions, and plateaued at two times blood pool reference value (e.g., 2 x [aorta reference uptake]). A first method (referred to as “P9050-sat”) reached the plateau when the plateau level was 50% of SUVₘₐₓ, the other (referred to as “P9040-sat”) reached the plateau when it was 40% of SUVₘₐₓ.

It was also found that refining automatically detected and segmented hotspots with thresholding changed a precision-recall tradeoff. While the original, automatically detected and segmented hotspots had high recall and low precision, refining segmentations with the P9050-sat thresholding method produced more balanced performance in terms of precision and recall.

Improved relative size preservation indicates that assessment of treatment response will be improved / more accurate, since the algorithm better captures the size order of the nuclear medicine reader’s annotations. Handling the tradeoff between over-segmentation and under-segmentation can be decoupled from the detection step by introducing a separate thresholding method - that is, using an analytical, adaptive segmentation approach as described herein, in addition to an automated hotspot detection and segmentation approach performed using machine learning approaches as described herein.

Example supporting studies described herein were used to determine scaling factors used to compute plateau values corresponding to maximum thresholds. For examine, as described herein, in the present example, such scaling factors were determined based on intensities in normal, healthy tissue in various reference regions. For example, multiplying a blood pool reference based on intensities in an aorta region by a factor of 1.6 produced a level that was typically above 95% of the intensity values in the aorta, but below typical normal uptake in the prostate. Accordingly, in certain example threshold functions, a higher value was used. In particular, in order to achieve a level that was also typically above most intensities in normal prostate tissue, a factor of 2 was determined. The value was determined manually based on investigations of histograms and image projections in sagittal, coronal, and transversal planes of PET image voxels within prostate volumes, but excluding any portions corresponding to tumor uptake. Example image slices and a corresponding histogram, showing the scaling factor, are shown in FIG. 18G.

### I. Introduction

Defining lesion volumes in PET/CT can be a subjective process, since lesions appear as hotspots in PET and typically do not have clear boundaries. Sometimes lesion volumes may be segmented based on their anatomical extent in a CT image, however this approach will lead to disregarding certain information about tracer uptake, since the full uptake will not be covered. Moreover, certain lesions may be visible in a functional, PET image, but cannot be seen in a CT image. This section describes an example study that designed thresholding methods aiming to accurately identify hotspot volumes reflecting a physiological uptake volume, i.e. a volume where uptake is above background. In order to perform segmentation and identify hotspot volumes in this manner, thresholds are selected so as to balance a risk of including background versus the risk of not segmenting a sufficiently large hotspot volume that reflects a full uptake volume.

This risk tradeoff is commonly addressed by selecting 50% or 40% of SUVₘₐₓ value determined for a prospective hotspot volume as the threshold. A rationale for this approach is that for high uptake lesions (e.g., corresponding to high intensity hotspots in a PET image) a threshold value can be set higher than for low uptake lesions (e.g., which correspond to lower intensity hotspots), while maintaining same risk level of not segmenting, as a hotspot volume, volume that represents the full uptake volume. However, for low signal-to-noise ratio hotspots, using a threshold value of 50% of SUVₘₐₓ will result in background being included in the segmentation. To avoid this, a decreasing percentage of SUVₘₐₓ can be used, starting, for example at 90% or 75% for low intensity hotspots. Moreover, risk of including background is low as soon as the threshold sufficiently above a background level, which occurs for threshold values well below 50% of SUVₘₐₓ for high uptake lesions. Accordingly, the threshold can be capped at a plateau level that is above typical background intensities.

One reference for an uptake intensity level that is well above typical background uptake intensity is average liver uptake . Other reference levels may be desirable, based on actual background uptake intensities. The background uptake intensity is different in bone, lymph and prostate, with bone having lowest background uptake intensity and prostate having highest background uptake intensity. Using a same thresholding method irrespective of tissue is advantageous / preferable, since it allows for a same segmentation method to be used without regard to a location and/or classification of a particular lesion. Accordingly, the study of this example evaluates thresholds using same threshold parameters for lesions in all three tissue types. The adaptive thresholding variants evaluated in this example include one that plateaus at liver uptake intensity, one that plateaus at a level estimated to be above aorta uptake, and several variants that plateaus at a level estimated to be above prostate uptake intensity.

Certain previous approaches have determined levels as a function of mediastinal blood pool uptake intensity, computed as a mean of blood pool uptake intensity plus two times standard deviation of the blood pool uptake intensity(e.g., mean of blood pool uptake intensity + 2 x SD). However, this approach, which relies on an estimation of standard deviation can lead to unwanted errors and noise sensitivities. In particular, estimating standard deviation is much less robust than estimating the mean, and may be affected by noise, minor segmentation errors or PET/CT misalignment. A more robust way to estimate a level above blood uptake intensity uses a fixed factor times the mean or reference aorta value. To find an appropriate factor, distributions of uptake intensity in the aorta were studied and are described in this example. Normal prostate uptake intensity was also studied to determine an appropriate factor that can be applied to reference aorta uptake to compute a level that is typically above normal prostate intensities.

### II. Methods

### Thresholding Manual Annotations

This study used a subset of the data that contained only lesions with at least one other lesion of the same type in the same patient. This resulted in a dataset with 684 manually segmented lesion uptake volumes (278 in bone, 357 in lymph nodes, 49 in prostate) across 92 patients. Automatic refinement by thresholding was performed, and the output was compared to the original volumes. Performance was measured by a weighted average of rank correlations between refined volumes and original volumes within a patient and tissue type, with the weight given by the number of segmented hotspots volumes in the patient. This performance measure indicates whether the relative sizes between segmented hotspot volumes have been preserved, but disregards absolute sizes, which are subjectively defined since uptake volumes do not have clear boundaries. However, for a particular patient and tissue type, the same nuclear medicine reader made all annotations, and they can hence be assumed to have been made in a systematic manner, with a smaller lesion annotation actually reflecting a smaller uptake volume compared to a larger lesion annotation.

### Thresholding Automatically Detected Lesions

This study used the subset of the data that had not been used for training the machine learning modules used for hotspot detection and segmentation, resulting in a dataset with 285 manually segmented lesion uptake volumes (104 bone, 129 lymph, 52 prostate) across 67 patients. Precision and recall was measured between the refined (and unrefined) automatically detected volumes that matched manually segmented lesions (sensitivity 90-91% for bone, 92-93% for lymph, 94-98% for prostate). These performance measures quantify the similarity between the automatically detected and possibly refined hotspots, and the manually annotated hotspots.

### Blood Uptake

For 242 patients, the thoracic part of the aorta was segmented in the CT component using a deep learning pipeline. The segmented aorta volume was projected to PET space, and eroded 3 mm to minimize the risk of the aorta volume containing regions outside the aorta or in the vessel wall, while retaining as much of the uptake inside the aorta as possible. For the remaining uptake intensity, in each patient the quotient q = (aortaMEAN + 2 x aortaSD) / aortaMEAN was computed.

### Prostate Uptake

In 29 patients, normal uptake in the prostate was studied. The study was performed by utilizing segmented prostate volumes determined via a machine learning module. Uptake intensities in the manually annotated prostate lesions were excluded. The remaining uptake intensity, normalized against aorta reference uptake intensity, was visualized by histograms as well as maximum intensity projections in the axial, sagittal and coronal planes, see an example in FIG. 18G. The purpose of the maximum projections was to find explanations for outlying intensities in the histograms, especially intensities pertaining to bladder uptake - above maximum uptake intensity in healthy tissue.

### Thresholding Methods

Two baseline methods (fixed threshold at SUV=2.5 and relative threshold at 50% of SUVₘₐₓ) were compared to six variants of adaptive thresholds. The adaptive thresholds were defined using three threshold functions, each associated with a particular range of SUVₘₐₓ values. In particular:
(1) Low range threshold function: A first threshold function was used to compute threshold values for SUVₘₐₓ values in a low range. The first threshold function computed threshold values as a fixed (high) percentage of SUVₘₐₓ,
(2) Intermediate range threshold function: A second threshold function was used to compute threshold values for SUVₘₐₓ values in an intermediate range. The second threshold function computed threshold values as a linearly decreasing percentage of SUVₘₐₓ, capped at a maximal threshold equaling the threshold at the upper end of the range, and
(3) High range threshold functions: A high range threshold function was used to compute threshold values for SUVₘₐₓ values in a high range. The high range threshold function set threshold values at either a maximal fixed threshold (saturated thresholds), or a fixed (low) percentage of SUVₘₐₓ (non-saturated thresholds).

Exact parameters of the three above described threshold functions and ranges varied between the various adaptive thresholding algorithms, and are listed in Table 2 below.

TABLE 2

Ranges and parameters for threshold functions of adaptive threshold algorithms

| | | | |
|---|---|---|---|
| Adaptive thresholds | Low range | Intermediate range *capped at interval right end value | High range |
| P9050-sat | 90% SUVₘₐₓ < aorta => 90% of SUVₘₐₓ | 90% SUVₘₐₓ = aorta, to 50% SUVₘₐₓ = 2 x aorta => Interp. perc. of SUVₘₐₓ^{∗} | 50% SUVₘₐₓ > 2 x aorta => 2 x aorta |
| P9040-sat | 90% SUVₘₐₓ < aorta => 90% of SUVₘₐₓ | 90% SUVₘₐₓ = aorta, to 40% SUVₘₐₓ = 2 x aorta => Interp. perc. of SUVₘₐₓ^{∗} | 40% SUVₘₐₓ > 2 x aorta => 2 x aorta |
| P7540-sat | 75% SUVₘₐₓ < aorta => 75% of SUVₘₐₓ | 75% SUVₘₐₓ = aorta, to 40% SUVₘₐₓ = 2 x aorta => Interp. perc. of SUVₘₐₓ^{∗} | 40% SUVₘₐₓ > x aorta => 2 x aorta |
| P9050-non-sat | 90% SUVₘₐₓ < aorta => 90% of SUVₘₐₓ | 90% SUVₘₐₓ = aorta, to 50% SUVₘₐₓ = 2 x aorta => Interp perc. of SUVₘₐₓ^{∗} | 50% SUVₘₐₓ > 2 x aorta => 50 % of SUVₘₐₓ |
| A9050-sat | 90% SUVₘₐₓ < aorta => 90% of SUVₘₐₓ | 90% SUVₘₐₓ = aorta, to 50% SUVₘₐₓ = 1.6 x aorta => Interp. perc. of SUVₘₐₓ^{∗} | 50% SUVₘₐₓ > 1.6 x aorta => 1.6 x aorta |
| L9050s-sat | 90% SUVₘₐₓ < aorta => 90% of SUVₘₐₓ | 90% SUVₘₐₓ = aorta, to 50% SUVₘₐₓ = liver => Interp. perc. of SUVₘₐₓ^{∗} | 50% SUVₘₐₓ > liver => liver |

The interpolated percentage used in the intermediate SUVₘₐₓ range is computed in the following manner for P9050-sat:

$$p = 90 - \frac{\left(90 - 50\right) ⋅ \left(S U {V}_{m a x} - S U {V}_{l o w}\right)}{S U {V}_{h i g h} - S U {V}_{l o w}} ,$$

where 50% of SUV_{high} equals 2 x [aorta uptake intensity], and 90% of SUV_{low} equals aorta uptake intensity.

Interpolated percentages used in other adaptive thresholding algorithms are computed analogously. The threshold in the intermediate range is then:

$$t h r = m i n \left(p \% ⋅ S U {V}_{m a x} , 50 \% ⋅ S U {V}_{h i g h}\right)$$

and analogously for the other adaptive thresholding algorithms.

### III. Results

### Thresholding Manually Annotated Lesions

The highest weighted rank correlations (0.81) were obtained by the P9050-sat and the P9040-sat methods, with P7540-sat, A9050-sat and L9050-sat also providing high values. The relative, 50% of SUVₘₐₓ (0.37) and P9050-non-sat (0.61) thresholding approaches resulted in the lowest weighted rank correlations. A fixed threshold at SUV=2.5 resulted in rank correlation in between (0.74), lower than the majority of the adaptive thresholding approaches. Weighted rank correlation results for each of the threshold approaches are summarized in Table 3, below.

TABLE 3

Weighted average of rank correlations for thresholding approaches evaluated

| Threshold strategy | Weighted average of rank correlations |
|---|---|
| fixed, SUV = 2.5 | 0.74 |
| relative, 50% of SUVₘₐₓ | 0.37 |
| P9050-sat | 0.81 |
| P9040-sat | 0.81 |
| P7540-sat | 0.79 |
| P9050-non-sat | 0.61 |
| A9050-sat | 0.80 |
| L9050s-sat | 0.78 |

### Thresholding of Automatically Detected Lesions

Without refinement, the automatic hotspot detections had low precision (0.31-0.47) but high recall (0.83-0.92), indicating over-segmentation. A refinement with a relative, 50% of SUVₘₐₓ thresholding algorithm improved precision (0.70-0.77), but decreased recall to about 50% (0.44-0.58). Refinement with P9050-sat also improved precision (0.51-0.84), with less drop in recall (0.61-0.89), indicating a balance with less over-segmentation but more under-segmentation. P9040-sat performed similarly to P9050-sat in these regards, whereas L9050-sat has the highest precision (0.85-0.95) but the lowest recall (0.31-0.56). Tables 4a-e show full results for precision and recall.

TABLE 4a

Precision and recall values without analytical segmentation refinement

| No refinement | Precision | Recall |
|---|---|---|
| Bone hotspots | 0.38 | 0.92 |
| Lymph hotspots | 0.47 | 0.83 |
| Prostate hotspots | 0.31 | 0.93 |

TABLE 4b

Precision and recall values with refinement via a relative, 50% of SUVₘₐₓ threshold approach

| relative, 50% of SUVₘₐₓ | Precision | Recall |
|---|---|---|
| Bone hotspots | 0.74 | 0.58 |
| Lymph hotspots | 0.77 | 0.44 |
| Prostate hotspots | 0.70 | 0.51 |

TABLE 4c

Precision and recall values with adaptive segmentation using the P9050-sat implementation

| P9050-sat | Precision | Recall |
|---|---|---|
| Bone hotspots | 0.84 | 0.61 |
| Lymph hotspots | 0.70 | 0.67 |
| Prostate hotspots | 0.51 | 0.89 |

TABLE 4d

Precision and recall values with adaptive segmentation t using the P9040-sat implementation

| P9040-sat | Precision | Recall |
|---|---|---|
| Bone hotspots | 0.84 | 0.59 |
| Lymph hotspots | 0.71 | 0.66 |
| Prostate hotspots | 0.52 | 0.89 |

TABLE 4e

Precision and recall values with adaptive segmentation using the L9050-sat implementation

| L9050-sat | Precision | Recall |
|---|---|---|
| Bone hotspots | 0.95 | 0.31 |
| Lymph hotspots | 0.91 | 0.39 |
| Prostate hotspots | 0.85 | 0.56 |

### Support for Thresholding Methods: Blood Uptake

For the resulting quotients, qMEAN + 2 x qSD was 1.54, hence using a factor of 1.6 was determined to be a good candidate for achieving a threshold level above most blood uptake intensity values. In the example study, only three patients had aortaMEAN + 2 x aortaSD that was above 1.6 x aortaMEAN. The three outlying patients had q=1.64, 1.92 and 1.61, where the patient with a factor of 1.92 had an erroneous aorta segmentation spilling into the spleen, and the others had quotients close to 1.6.

### Support for Thresholding Methods: Prostate Uptake

Based on a manual review of histograms of normal prostate intensities with the projections in the axial, sagittal and coronal planes in mind, a value of 2.0 would be an appropriate scaling factor to apply to the aorta reference value to get a level that was above typical uptake intensity in the prostate.

### H. Example: Use of AI-Based Hotspot Segmentation in Comparison with Thresholding Alone

In this example, hotspot detection and segmentation performed using an AI-based approach that utilizes machine learning modules to segment and classify hotspots as described herein was compared with a conventional approach that utilized a threshold-based segmentation alone.

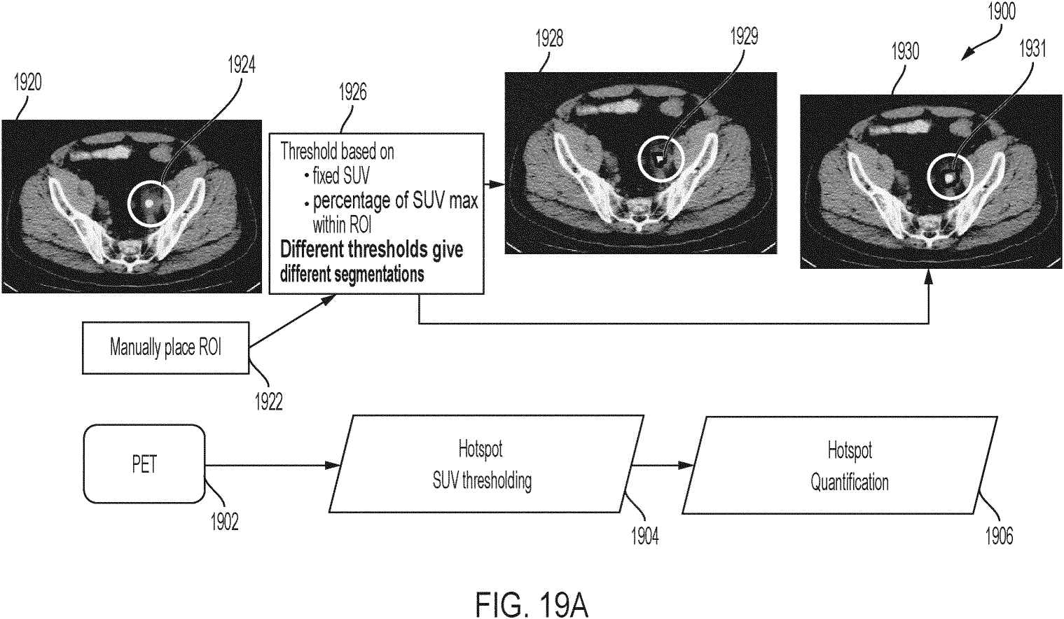

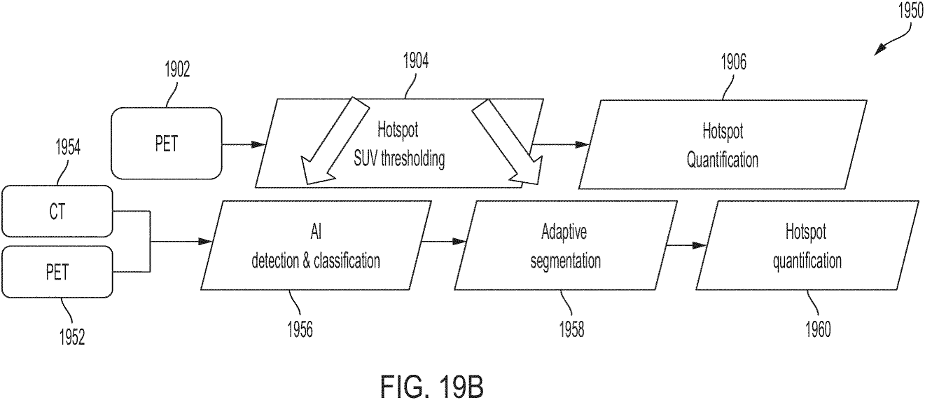

FIG. 19A shows a conventional hotspot segmentation approach 1900, which does not utilize machine learning techniques. Instead, hotspot segmentation is performed based on a manual delineation of hotspots, by a user, followed by intensity (e.g., SUV) - based thresholding 1904. A user manually masks places 1922 a circular marker indicating a region of interest (ROI) 1924 within an image 1920. Once the ROI is placed, either a fixed or relative threshold approach may be used to segment a hotspot within the manually placed ROI 1926. The relative threshold approach sets a threshold for a particular ROI as a fixed percentage of a maximum SUV within the ROI., individually, and an SUV-based thresholding approach is used to segment each user-identified hotspot, refining the initial user-drawn boundary. Since this conventional approach relies on a user manually identifying and drawing boundaries of hotspots, it can be time consuming and, moreover, segmentation results, as well as downstream quantification 1906 (e.g., computation of hotspot metrics) can vary from user to user. Moreover, as illustrated conceptually in images 1928 and 1930, depending on a particular threshold, different thresholds may produce different hotspot segmentations 1929, 1931. Additionally, while SUV threshold levels can be tuned to detect early-stage disease, doing so often results in a high number of false positive findings, distracting from true positives. Finally, for example as explained herein, conventional fixed or relative SUV-based thresholding approaches suffer from over and/or under-estimation of lesion size.

Turning to FIG. 19B, instead of utilizing a manual, user-based selection of ROIs containing hotspots in connection with SUV-based thresholding, an AI-based approach 1950 in accordance with certain embodiments described herein utilizes one or more machine learning modules to automatically analyze a CT 1954 and a PET image 1952 (e.g., of a composite PET/CT) to detect, segment, and classify hotspots 1956. As described in further detail herein, machine learning-based hotspot segmentation and classification can be used to create an initial 3D hotspot map, which can then be used as an input for an analytical segmentation method 1958, such as the adaptive thresholding technique described herein, for example in Sections F and G. Use of machine learning approaches, among other things, reduces user subjectivity and time needed to review images (e.g., by medical practitioners, such as radiologists). Moreover, AI models are capable of performing complex tasks, and can identify early-stage lesions as well as high-burden metastatic disease, while keeping false positive rate low. Improved hotspot segmentation in this manner improves accuracy of downstream quantification 1960 relevant for measuring, among other things, metrics that can be used to assess disease severity, prognosis, treatment response and the like.

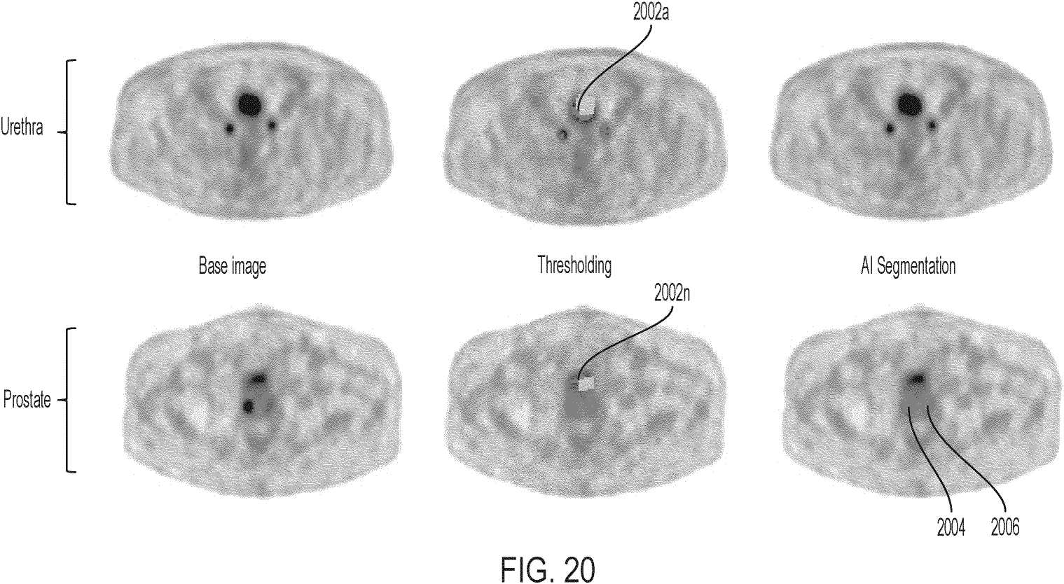

FIG. 20 demonstrates improved performance of a machine learning based segmentation approach in comparison with a conventional thresholding method. In the machine learning based approach, hotspot segmentation was performed by first detecting and segmenting hotspots using machine learning modules, as described herein (e.g., in section E), along with refinement using an analytical model that implemented a version of the adaptive thresholding technique described in Sections F and G. The conventional thresholding method was performed using a fixed thresholding, segmenting clusters of voxels having intensities above the fixed threshold. As shown in FIG. 20, while a conventional thresholding method generates false positives 2002a and 2002b due to uptake of radiopharmaceutical in a urethra, the machine learning segmentation technique correctly ignores urethra uptake and segments only prostate lesions 2004 and 2006.

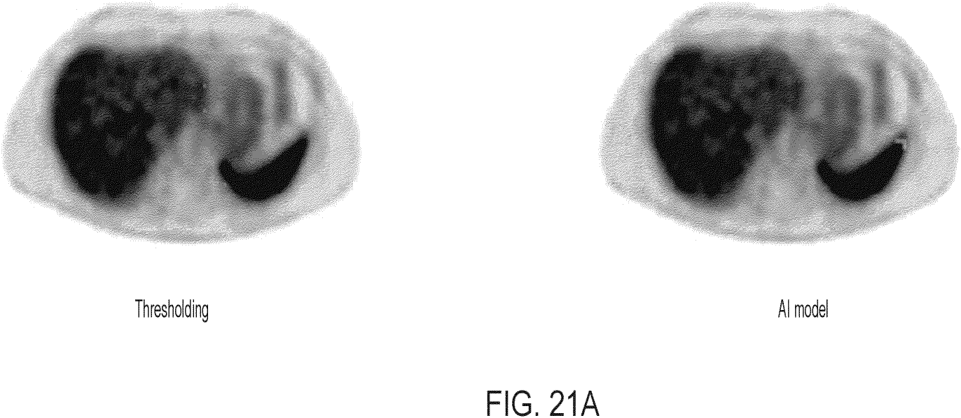

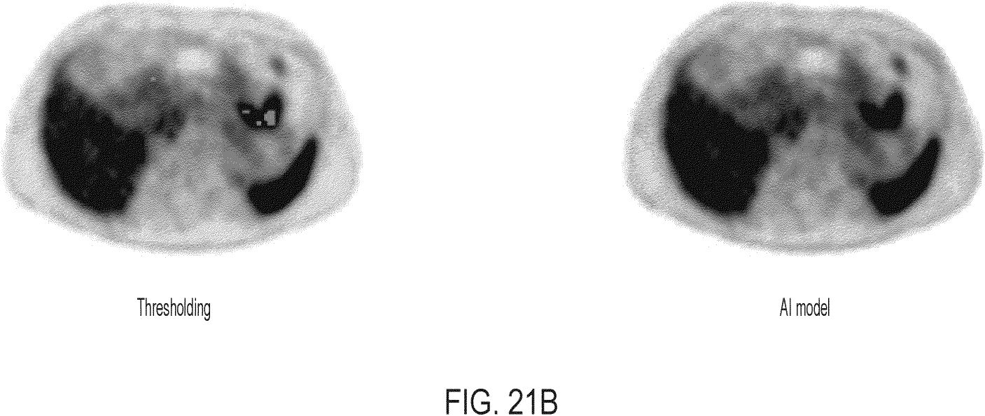

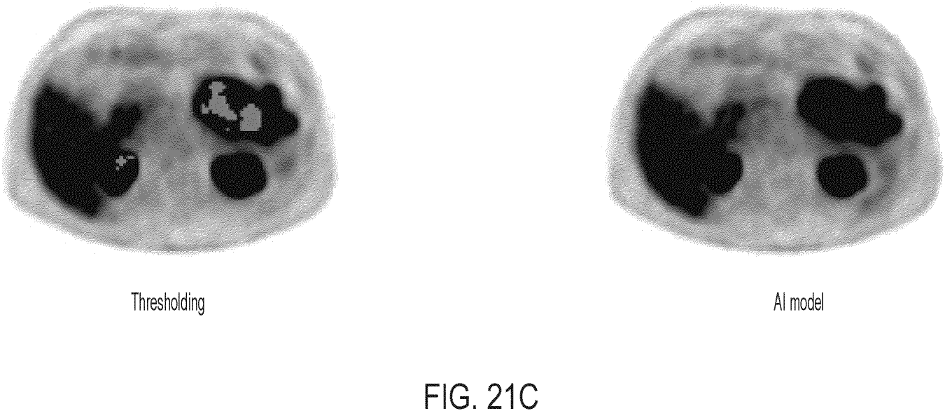

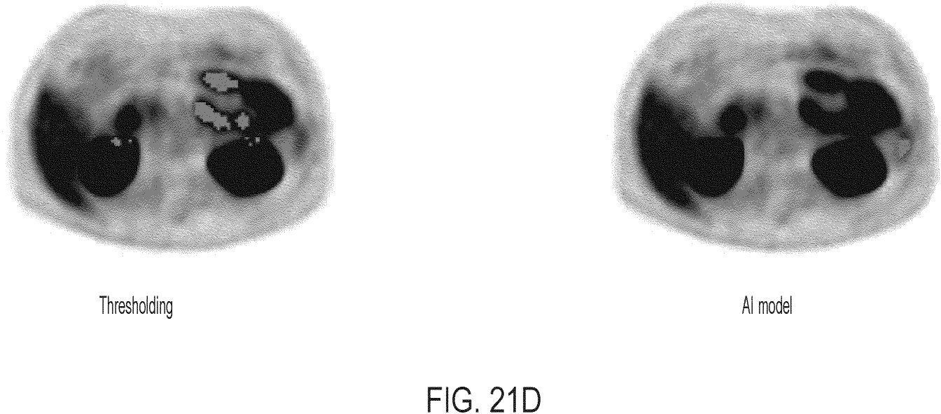

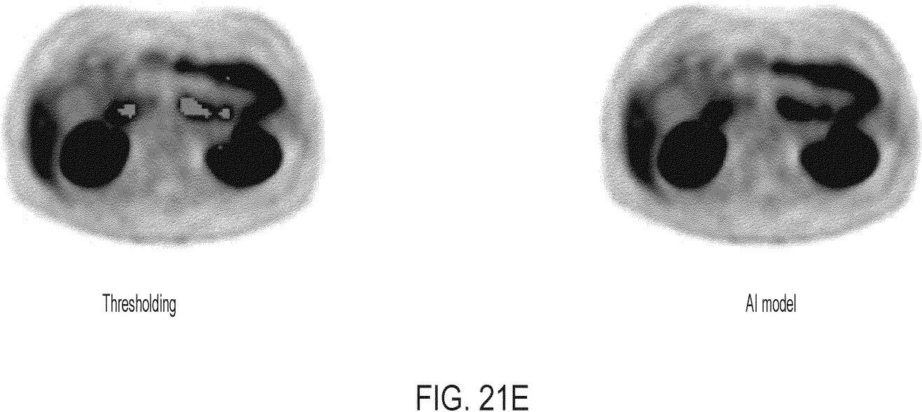

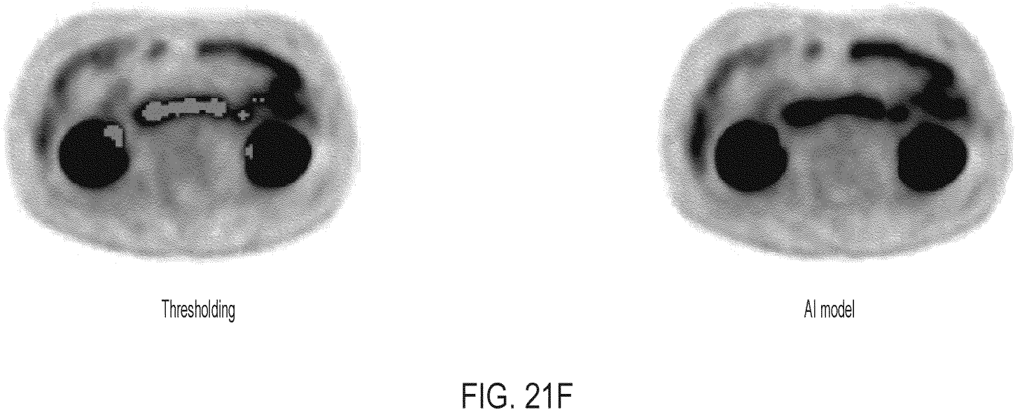

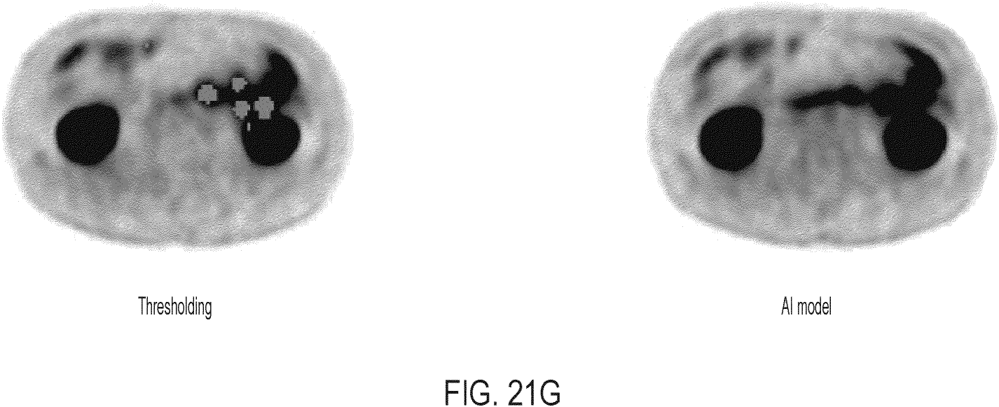

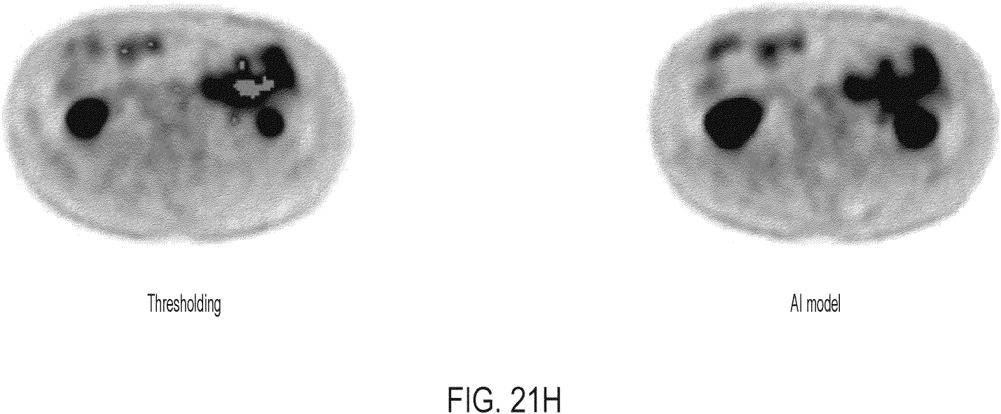

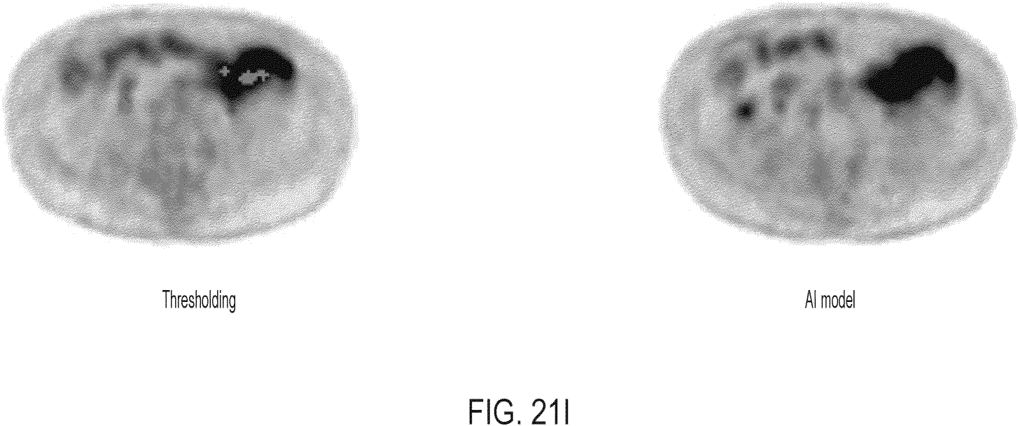

FIGS. 21A-I compare hotspot segmentation results within an abdominal region performed by a conventional thresholding method (left hand images) with those of a machine learning approach in accordance with embodiments described herein (right hand images). FIGS. 21A-I show a series of 2D slices of a 3D image, moving along a vertical direction in an abdominal region, with hotspot regions identified by each method overlaid. The results shown in the figures show that abdominal uptake is a problem for the conventional thresholding approach, with large false positive regions appearing in the left hand side images. This may result from large uptake in kidneys and bladder. Conventional segmentation approaches requires complex methods to suppress this uptake and limit such false positives. In contrast, the machine learning model used to segment images shown in FIGS. 21A-I did not rely on any such suppression, and instead learned to ignore this kind of uptake.

### I. Example CAD Device Implementation

This section describes an example CAD device implementation in accordance with certain embodiments described herein. The CAD device described in this example is referred to as “aPROMISE” and performs automated organ segmentation using multiple machine learning modules. The example CAD device implementation uses analytical models to perform hotspot detection and segmentation.

The aPROMISE (automated PROstate specific Membrane Antigen Imaging SEgmentation) example implementation described in this example utilizes a cloud-based software platform with a web interface where users can upload body scans of PSMA PET/CT image data in the form of DICOM files, review patient studies and share study assessments within a team. The software complies with the Digital Imaging and Communications in Medicine (DICOM) 3 standard. Multiple scans can be uploaded for each patient and the system provides a separate review for each study. The software includes a GUI that provides a review page that displays and allows a user to view studies in a 4-panel view showing PET, CT, PET/CT fusion and maximum intensity projection (MIP) simultaneously, and includes an option to display each view separately. The device is used to review entire patient studies, using image visualization and analysis tools for users to identify and mark regions of interest (ROIs). While reviewing image data, users can mark ROIs by selecting from pre-defined hotspots that are highlighted when hovering with the mouse pointer over the segmented region, or by manual drawing, i.e selecting individual voxels in the image slices to include as hotspots. Quantitative analysis is automatically performed for selected or (manually) drawn hotspots. The user can review the results of this quantitative analysis and determine which hotspots should be reported as suspicious lesions. In aPROMISE, Region of interest (ROI) refers to a contiguous sub-portion of an image; Hotspot refers to a ROI with high local intensity (e.g., indicative of high uptake) (e.g., relative to surrounding areas), and Lesion refers to a user defined or user selected ROI that is considered suspicious for disease.

To create a report the software of the example implementation requires a signing user to confirm quality control, and electronically sign the report preview. Signed reports are saved in the device and can be exported as a JPG or DICOM file.

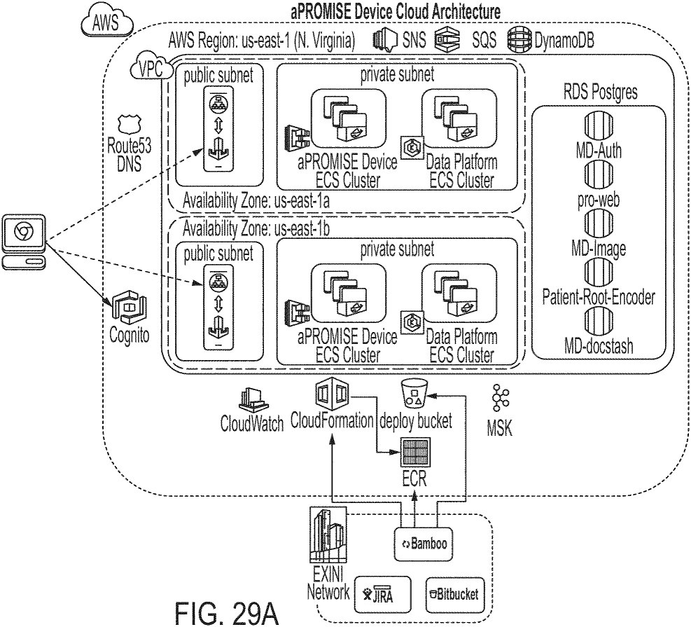

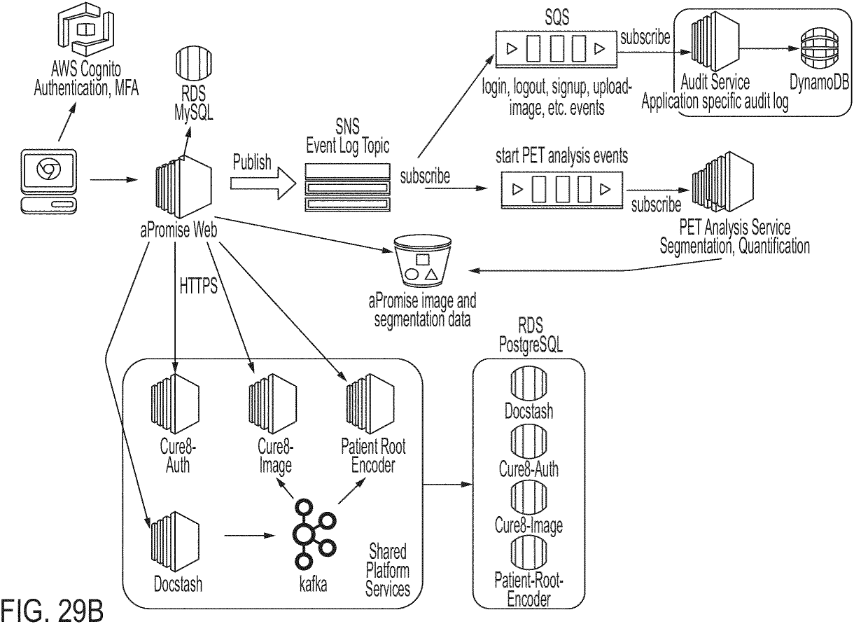

The aPROMISE device is implemented in a microservice architecture, as described in further detail herein and shown in FIGS. 29A and 29B.

### I. Workflow

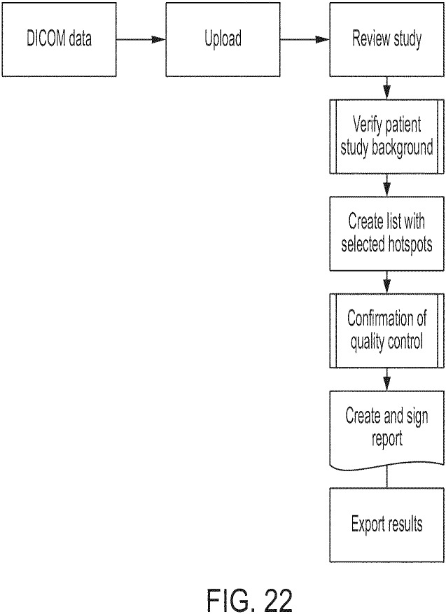

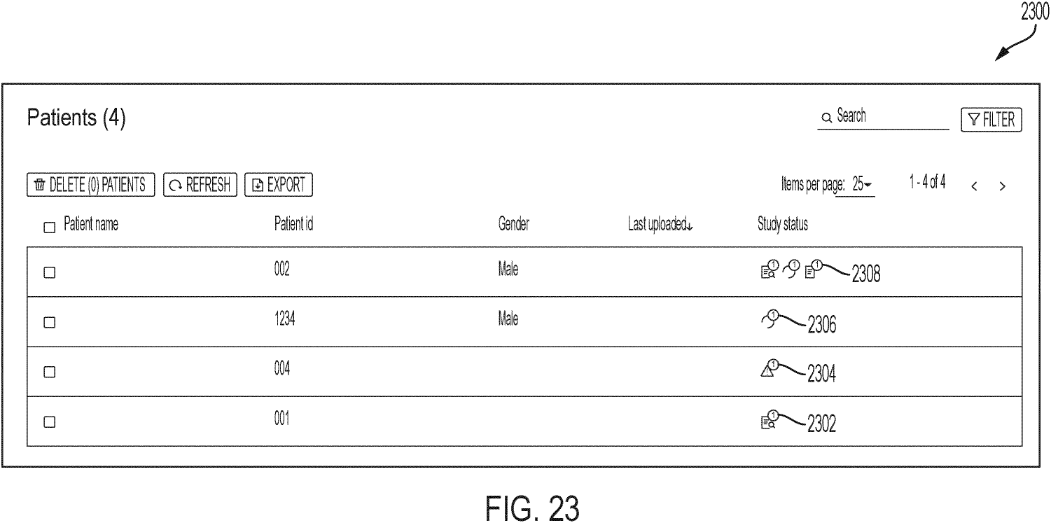

FIG. 22 depicts workflow of the aPROMISE device from uploading DICOM files to exporting electronically signed reports. When logged in, a user can import DICOM files into aPROMISE. Imported DICOM files are uploaded to the patient list, where the user can click on a patient to display the corresponding studies available for review. The layout principles for the patient list are displayed in FIG. 23.

This view 2300 lists all patients with uploaded studies within a team and displays patient information (name, ID and gender), latest study upload date, and study status. The study status indicates if studies are ready for review (blue symbol, 2302), studies with errors (red symbol, 2304), studies calculating (orange symbol, 2304) and studies with reports available (black symbol, 2308) per patient. The number in the top right corner of the status symbol indicates the number of studies with a specific status per patient. The review of a study is initiated by clicking on a patient, selecting a study and identifying if the patient has had a prostatectomy or not. The study data will be opened and displayed in a review window.

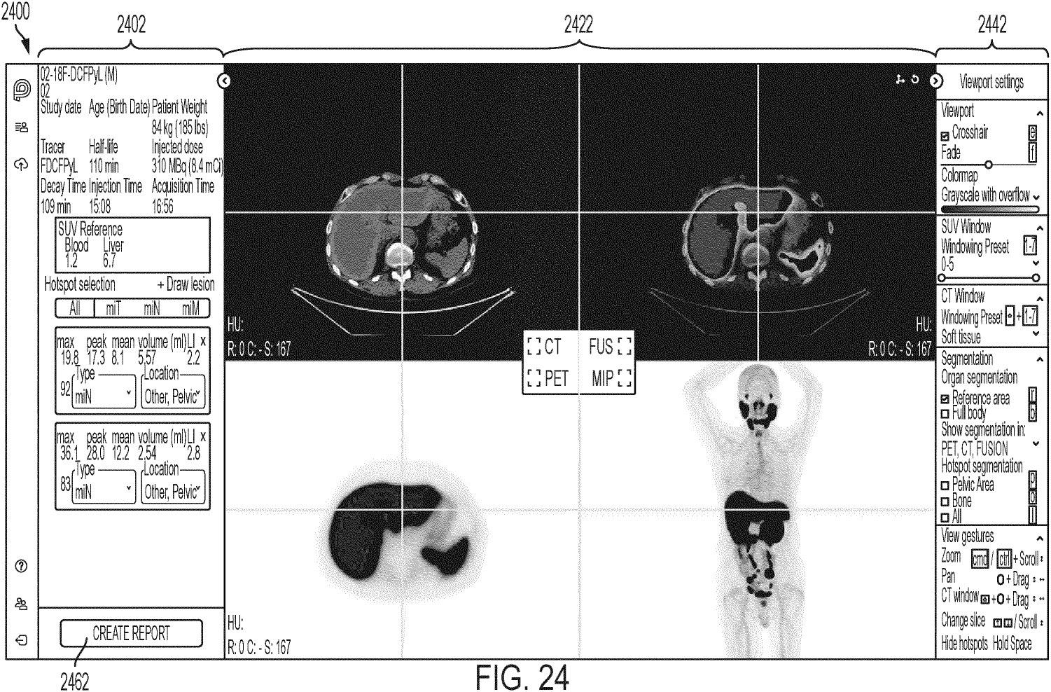

FIG. 24 shows review window 2400, where the user can examine the PET/CT image data. Lesions are manually marked and reported by the user, who either selects from pre-defined hotspots, segmented by the software, or user-defined hotspots made by using the drawing tool for selecting voxels to include as hotspots in the program. Predefined hotspots, regions of interest with high local intensity uptake, are automatically segmented using specific methods for soft tissue (prostate and lymph nodules) and bone, and are highlighted when hovering with the mouse pointer over the segmented region. The user can choose to turn on a segmentation display option to visually present the segmentations of pre-defined hotspots simultaneously. Selected or drawn hotspots are subject for automatic quantitative analysis and are detailed in panels 2402, 2422, and 2442.

Retractable panel 2402 on the left summarizes patient and study information that are extracted from DICOM data. Panel 2402 also displays and lists quantitative information about the hotspots that are selected by the user. The hotspot location and type are manually verified - T: localized in the primary tumor, N: Regional metastatic disease, Ma/b/c: Distant metastatic disease (lymph node, bone and soft tissue). The device displays the automated quantitative analysis - SUV-max, SUV-peak, SUV-mean, Lesion Volume, Lesion Index (LI) - on the user selected hotspots, allowing the user to review and decide on which hotspots to report as lesions in a standardized report.

Middle Panel 2422 includes a four panel-view display of the DICOM image data. Top left corner displays the CT image, top right displays the PET/CT fusion view, bottom left displays the PET image and the bottom right show the MIP.

MIP is a visualization method for volumetric data that displays a 2D projection of a 3D image volume from various view angles. MIP imaging is described in Wallis JW, Miller TR, Lerner CA, Kleerup EC. Three-dimensional display in nuclear medicine. IEEE Trans Med Imaging. 1989;8(4):297-30. doi: 10.1109/42.41482. PMID: 18230529.

Retractable right panel 2442 comprises the following visualization controls for optimizing image review and its shortcut keys to manipulate the image for review purposes:

Viewport:
presence or absence of crosshairs
fading option for the PET/CT fusion image
selection of which standard nuclear medicine colormap to visualize the PET tracer uptake intensities.

SUV and CT Window:
Windowing of the images, also known as contrast stretching, histogram modification or contrast enhancement, where images are manipulated via the intensity, change the appearance of the picture to highlight particular structures.
In the SUV Window, windowing presets for SUV intensities can be adjusted by the slider or shortcut keys.
In the CT Window, window presets for Hounsfield intensities can be selected from a drop-down list, using the shortcut keys or by a click-and-drag input, where brightness of the image is adjusted via the window level and contrast is adjusted via the window width.

Segmentation
The organ segmentation display options to turn on or off the visualization of the segmentation of the reference organs or the full body segmentation.
The user can select which panel-views to display the organ segmentation.
The hotspot segmentation display options to turn on or off the presentation of pre-defined hotspots in selected areas; the pelvic area, in bones or all hotspots.

Viewer gestures
Shortcut keys and combinations for Zoom, Pan, CT window, Change slice and Hide hotspots of the review window.

To proceed with the report creation, a signing user clicks on Create Report button 2462. The user must confirm the following quality control items before a report will be created:
Image quality is acceptable
PET and CT images are correctly aligned
Patient study data is correct
Reference values (blood pool, liver) are acceptable
Study is not a superscan

Following confirmation of the quality control items, a preview of the report is shown for electronic signing by the user. The report includes the patient summary, the total quantitative lesion burden, and the quantitative assessment of individual lesions from the user selected hotspots, to be confirmed as lesions by the user.

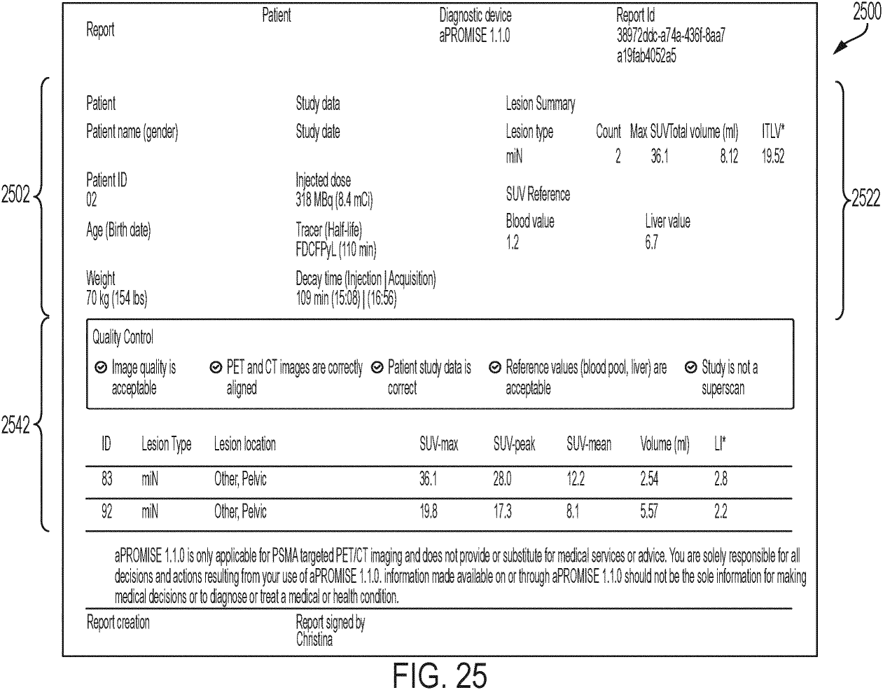

FIG. 25 shows an example generated report 2500. Report 2500 includes three sections, 2502, 2522, and 2542.

Section 2502 of report 2500 provides a summary of patient data obtained from the DICOM tags. It is includes a summary of the Patient; Patient name, Patient ID, Age and Weight, and a summary of the Study data; Study date, injected dose at the time of injection, the radiopharmaceutical imaging tracer used and its half-life, and the time between injection of tracer and acquisition of image data.

Section 2522 of report 2500 provides summarized quantitative information from the hotspots selected by the user to be included as lesions. The summarized quantitative information displays the total lesion burden per lesion type (primary prostate tumor (T), local/regional pelvic lymph node (N) and distant metastasis - lymph node, bone or soft tissue organs (Ma/b/c)). The summary section 2522 also displays the quantitative uptake (SUV-mean) that was observed in the reference organs.

Section 2542 of report 2500 is the detailed quantitative assessment and location of each lesion, from the selected hotspots confirmed by the user. Upon reviewing the report, the user must electronically sign his/her patient study review results, including selected hotspots and quantifications as lesions. Then the report is saved in the device and can be exported as a JPG or DICOM file.

### II. Image Processing

### Preprocessing of DICOM Input Data

Image input data is presented in DICOM format which is a rich data representation. DICOM data includes intensity data as well as meta data and a communication structure. In order to optimize the data for aPROMISE usage, the data is passed through a microservice that re-encodes, compresses and removes unnecessary or sensitive information. It also gathers intensity data from separate DICOM series and encode the data into a single lossless PNG file with an associated JSON meta information file.

Data processing of PET image data includes estimation of a SUV (Standardized Uptake Value) factor, which is included in the JSON meta information file. The SUV factor is a scalar used to translate image intensities into SUV values. The SUV factor is calculated according to QIBA guidelines (Quantitative Imaging Biomarkers Alliance).

### Algorithm Image Processing

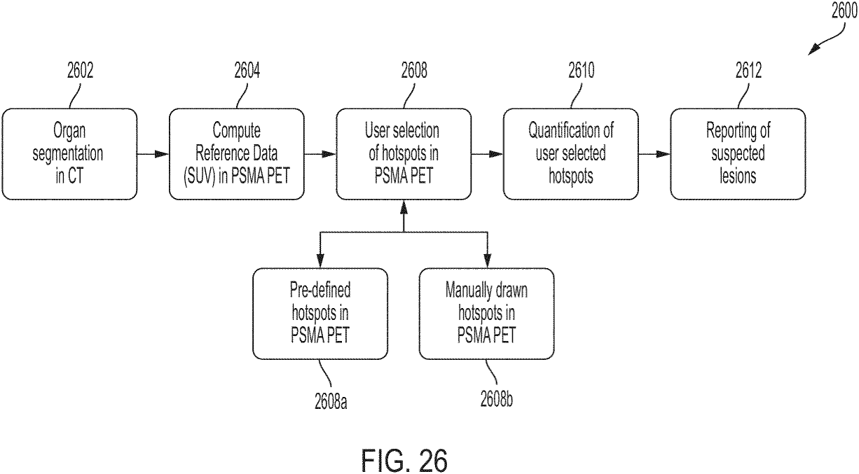

FIG. 26 shows an example image processing workflow (process) 2600.

aPROMISE uses a CNN (convolutional neural network) model to segment 2602 a patient skeleton and selected organs. Organ segmentation 2602 allows for automated calculation of the standard uptake value (SUV) reference in an aorta and liver of the patient 2604. The SUV-reference for the aorta and liver are then used as reference values when determining certain SUV-value based quantitative indices, such as Lesion Index (LI) and intensity-weighted tissue lesion volume (ITLV). A detailed description of the quantitative indices is provided in Table 6, below.

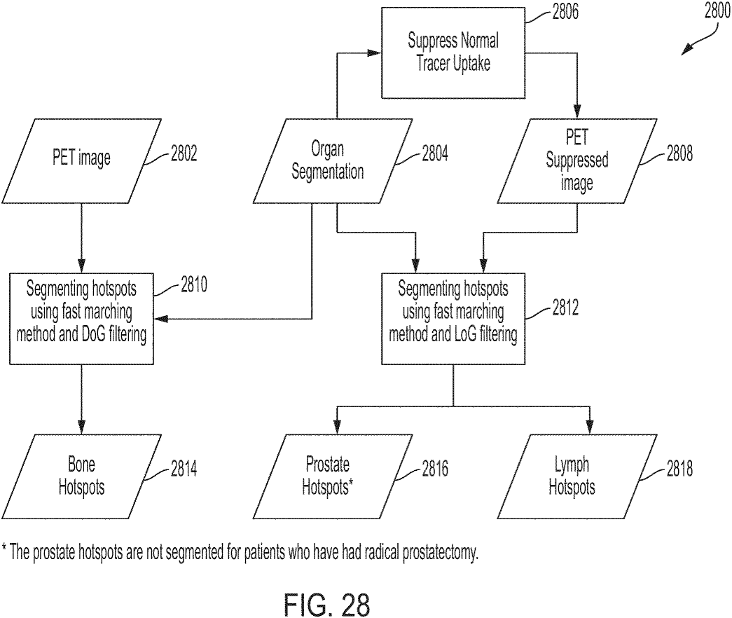

Lesions are manually marked and reported by the user 2608, who either selects from pre-defined hotspots 2608a, segmented by the software, or user-defined hotspots made by using the drawing tool 2608b for selecting voxels to include as hotspots within the GUI. Pre-defined hotspots, regions of interest with high local intensity uptake, are automatically segmented using certain particular methods for soft tissue (prostate and lymph nodules) and bone (e.g., as shown in FIG. 28, one particular segmentation method for bone and another for soft tissue regions may be used). Based on the organ segmentation, the software determines a type and location for selected hotspots in prostate, lymph or bone regions. Determined type and locations are displayed in a list of selected hotspots shown in panel 2502 of viewer 2400. Type and location of selected hotspots in other regions (e.g., not located in prostate, lymph, or bone regions) are manually added by the user. The user can add and edit the type and locations of all hotspots as applicable at any time during the hotspot selection. The hotspot type is determined using the miTNM system, which is a clinical standard and a notation system for reporting of the spreading of cancer. In this approach, individual hotspots are assigned type according to a letter based code that indicates certain physical features as follows:
T indicates the primary tumor
N indicates lymph nodes nearby that are affected by the primary tumor
M indicates distant metastasis

For distant metastasis lesions, localizations are grouped into the a/b/c-system corresponding to extra pelvic lymph nodes (a), bones (b) and soft tissue organs (c).

For all hotspots selected to be included as lesions, SUV-values and indices are calculated 2610 and displayed in the report.

### Organ Segmentation in CT

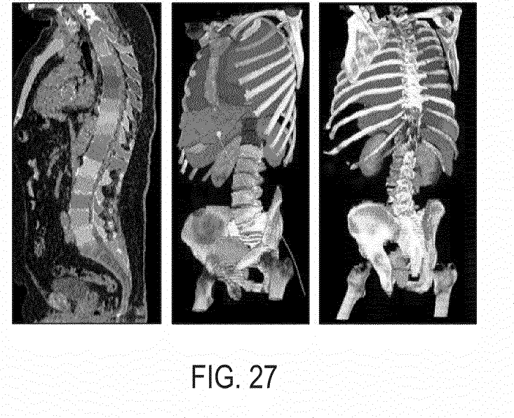

The organ segmentation 2602 is performed using the CT-image as an input. Starting with two coarse segmentations from the full image, smaller image sections are extracted, and selected to contain a given set of organs. A fine segmentation of organs is performed on each image section. Finally, all segmented organs from all image sections are assembled into the full image segmentation displayed in aPROMISE. A successfully completed segmentation identifies 52 different bones and 13 soft tissue organs as visualized in FIG. 27, and presented in Table 5. Both the coarse and fine segmentation processes include three steps:
1. Preprocessing of the CT image,
2. CNN segmentation, and
3. Postprocessing the segmentation.

Preprocessing the CT-image prior to the coarse segmentation includes three steps: (1) removing image slices that represent only air (e.g., having <= 0 Hounsfield Units), (2) re-sampling the image to a fixed size, and (3) normalizing the image based on the mean and standard deviation for the training data, as described below.

The CNN-models performs semantic segmentation where each pixel in the input image is assigned a label corresponding to either background or the organ it segments, resulting in a label map of the same size is the input data.

Postprocessing is performed after the segmentation and includes the following steps:
Absorbing neighboring pixel clusters once.
Absorbing neighboring pixel clusters until no such cluster exists.
Removing all clusters that are not the largest of each label.
Discard skeletal parts from the segmentation; Some segmentation models segment skeletal parts as reference points when segmenting soft tissue. The skeletal parts in these models are meant to be removed after segmentation is done.

Two different coarse segmentation neural networks and ten different fine segmentation neural networks are used, including the segmentation of the prostate. If the patient has undergone a prostatectomy prior to the examination - information provided by the user when verifying the patient study background before opening a study for review - then the prostate is not segmented. The combination of the fine and coarse segmentation and which body part each combination provides are presented in Table 5.

TABLE 5

A summary of how the coarse and fine segmentation networks are combined to segment different body parts

| | | |
|---|---|---|
| Organs/Bones | Coarse Segmentation Neural Network | Fine Segmentation Neural Network |
| Right Lung | coarse-seg-02 | fine-seg-right-lune-01 |
| Left Lung | coarse-seg-02 | fine-seg-left-lung-01 |
| Left/Right Femur Left/Right Gluteus Maximus | coarse-seg-04 | fine-seg-legs-01 |
| Left/Right Hip Bone | coarse-seg-04 | fine-seg-pelvic-noprostate-01 |
| Sacrum and coccyx | | |
| Urinary bladder | | |
| Liver | coarse-seg-02 | fine-seg-abdomen-02 |
| Left/Right Kidney | | |
| Gallbladder | | |
| Spleen | | |
| Right Ribs 1-12 | coarse-seg-02 | fine-seg-right-upper-body-bone-02 |
| Right Scapula | | |
| Right Clavicle | | |
| Left Ribs 1-12 | coarse-seg-02 | fine-seg-left-upper-body-bone-02 |
| Left Scapula | | |
| Left Clavicle | | |
| Cervical vertebrae | coarse-seg-02 | fine-seg-spinebone-02 |
| Thoracic vertebrae 1-12 | coarse-seg-02 | fine-seg-aorta-01 |
| Lumbar vertebrae 1-5 | | |
| Sternum | | |
| Aorta, thoracic part | | |
| Aorta, abdominal part | | |
| Prostate^{∗} | coarse-seg-02 | fine-seg-pelvic-region-mixed |

^{∗}Additional segmentation network only applicable for patients with a prostate.

Training the CNN models includes an iterative minimization problem where the training algorithm updates model parameters to lower the segmentation error. Segmentation error is defined as the deviation from a perfect overlap between manual segmentation and the CNN-model segmentation. Each neural network used for organ segmentation was trained to configure optimal parameters and weights. The training data for developing the neural networks for aPROMISE, as described above, consists of low dose CT images with manually segmented and labelled body parts. The CT images for training segmentation network were gathered as a part of the NIMSA project (http://nimsa.se/) and during a phase II clinical trial of the drug candidate 99mTc-MIP-1404 registered at clinicaltrials.gov (https://www.clinicaltrials.gov/ct2/show/NCT01667536?term=99mTc-MIP-1404&draw=2&rank=5). The NIMSA project consists of 184 patients and the 99mTc-MIP-1404-data consists of 62 patients.

### Compute Reference Data (SUV Reference) in PSMA PET

Reference values are used when evaluating the physiological uptake of the PSMA tracer. The current clinical praxis is to use the SUV intensities in identified volumes corresponding to either the blood pool or in the liver or both tissues as a reference value. For PSMA tracer intensity, the blood pool is measured in an aorta volume.

In aPROMISE SUV intensities in volumes corresponding to the thoracic part of the aorta and liver are used as reference values. The uptake registered in the PET image together with the organ segmentation of aorta and liver volumes are the basis for calculating the SUV reference in the respective organ.

Aorta. To ensure that portions of the image corresponding to the vessel wall are not included in the volume used to calculate the SUV reference for the aorta region, the segmented aorta volume is reduced. The segmentation reduction (3 mm) was heuristically selected to balance the tradeoff of keeping as much of the aorta volume as possible while not including the vessel wall regions. The reference SUV for the blood pool is a robust average of SUV from pixels inside the reduced segmentation mask identifying the aorta volume. The robust average is computed as the mean of the values in the interquartile range.

Liver. When measuring the reference value in the liver volume, the segmentation is reduced along edges to create a buffer adjusting for possible misalignment between the PET and CT images. The reduction amount (9 mm) was determined heuristically using manual observations of images with PET/CT misalignment.

Cysts or malignancies in the liver can result in regions of low tracer uptake in the liver. To reduce impact from these local differences in tracer uptake to the calculation of the SUV reference, a two-component Gaussian mixture model approach, in accordance with embodiments described in Section B.iii, above, with regard to FIG. 2A was used. In particular, a two-component Gaussian mixture model was fit to the SUVs from voxels inside the reference organ mask and a major and minor component of a the distribution identified. The SUV reference for the liver volume was initially computed as the average SUV of the major component from the Gaussian mixture model. If the minor component was determined to have a larger average SUV than the major component, the liver reference organ mask is kept unchanged, unless a weight of the minor component is more than 0.33 - in this case, when a weight of the minor component was more than 0.33, an error is thrown and the liver reference value will not be calculated.

If the minor component has a smaller average SUV than the major component, a separation threshold is computed, for example as shown in FIG. 2A. The separation threshold is defined so that:
The probability to belong to the major component for a SUV at the threshold or larger, and;
The probability to belong to the minor component for a SUV at the threshold or smaller;
are equal.

The reference mask is then refined by removing the pixels below the separation threshold.

### Pre-Definition of Hotspots in PSMA PET

Turning to FIG. 28, in the aPROMISE implementation of the present example, segmentation of regions with high local intensity in PSMA PET by aPROMISE, so called pre-defined hotspots, is performed by an analytical model 2800 based on input from PET images 2802 and the organ segmentation map 2804 determined from the CT image and projected in the PET space. For the software to segment hotspots in bone, the original PET images 2802 are used and for segmenting hotspots in lymph and prostate the PET image is processed by suppressing the normal PET tracer uptake 2806. A graphical overview of an analytical model, used in this example implementation, is presented in FIG. 28. The analytical method, as further explained below, was designed to find the high local uptake intensity regions that may represent ROIs without an excessive number of irrelevant regions or PET tracer background noise. The analytical method was developed from a labeled data set comprising of PSMA PET/CT images.

The suppression 2806 of the normal PSMA tracer uptake intensity was performed in one high-uptake organ at the time. First, the uptake intensity in the kidneys is suppressed, then the liver and finally the urinary bladder. The suppression is performed by applying an estimated suppression map to the high-intensity regions of the PET. The suppression map is created using the organ map previously segmented in the CT and projecting and adjusting it to the PET image, creating a PET adjusted organ mask.

The adjustment corrects for small misalignments between the PET and CT images. Using the adjusted map, a background image is calculated. This background image is subtracted from the original PET image and creates an uptake estimation image. The suppression map is then estimated from the uptake estimation image using an exponential function that is dependent on a Euclidean distance from a voxel outside the segmentation to the PET adjusted organ mask. An exponential function is used since the uptake intensity decreases exponentially with distance from the organ. Finally, the suppression map is subtracted from the original PET image, thereby suppressing intensities associated with high normal uptake in the organ.

After suppression of the normal PSMA tracer uptake intensity, hotspots are segmented in the prostate and lymph 2812 using organ segmentation mask 2804 and suppressed PET image 2808 created by suppression step 2806. The prostate hotspots are not segmented for patients who have had prostatectomy. Bone and lymph hotspot segmentations are applicable for all patients. Each hotspot is segmented using a fast-marching method where the underlying PET image is used as the velocity map and the volume of an input region determines a travel time. The input region is also used as an initial segmentation mask to identify a volume of interest for the fast-marching method and is created differently depending on whether hotspot segmentation is performed in bone or soft tissue. Bone hotspots are segmented using a fast marching method and Difference of Gaussian (DoG) filtering approach 2810 and lymph and, if applicable, prostate hotspots are segmented using a fast marching method and Laplacian of Gaussian (LoG) filtering approach 2812.

For detection and segmentation of bone hotspots, a skeletal region mask is created to identify a skeletal volume, in which bone hotspots may be detected. The skeletal region mask is comprised of the following skeletal regions: Thoracic vertebrae (1-12), Lumbar vertebrae (1-5), Clavicles (L+R), Scapulae (L+R), Sternum Ribs (L+R, 1-12), Hip bones (L+R), Femurs (L+R), Sacrum and Coccyx. The masked image is normalized based on a mean intensity of the healthy bone tissue in PET image, performed by iteratively normalizing the image using DoG filtering. Filter sizes used in the DoG are 3 mm/spacing and 5 mm/spacing. The DoG filtering acts as a band-pass filter on the image that impairs signal further away from the band center, which emphasizes clusters of voxels with intensities that are high relative to their surroundings. Thresholding the normalized image obtained in this manner produces clusters of voxels which may be differentiated from background and, accordingly, segmented, thereby creating a 3D segmentation map 2814 that identifies hotspot volumes located in bone regions.

For detection and segmentation of lymph hotspots, an lymph region mask is created in which hotspots corresponding to potential lymph nodules may be detected. The lymph region mask includes voxels that are within a bounding box that encloses all segmented bone and organ regions, but excludes voxels within the segmented organs themselves, apart from lungs volumes, voxels of which are retained. Another, prostate region mask is created in which hotspots corresponding to potential prostate tumors may be detected. This prostate region mask is a one voxel dilation of a prostate volume determined from the organ segmentation step described herein. Applying the lymph region mask to the PET image creates a masked image that includes voxels within the lymph region (e.g., and excludes other voxels) and, likewise, applying the prostate region mask to the PET image creates a masked image that includes voxels within the prostate volume.

Soft tissue hotspots - i.e., lymph and prostate hotspots - are detected by separately applying three different sizes LoG filters - one with 4 mm/spacingXYZ, one with 8 mm/spacingXYZ, and one with 12 mm/spacingXYZ - on the lymph and/or prostate masked images, thereby creating three LoG filtered images for each of the two soft tissue types (prostate and lymph). For each soft-tissue type, the three corresponding LoG filtered images are thresholded using a value of minus 70% of aorta SUV reference and then local minima are found using a 3×3×3 minimum filter. This approach creates three filtered images, each comprising clusters of voxels corresponding to hotspots. The three filtered images are combined by taking the union of the local minima from the three images to produce, a hotspot region mask. Each component in the hotspot region mask is segmented using a level set-method to determine a one or more hotspot volumes. This segmentation approach is performed for both prostate and for lymph hotspots, thereby automatically segmenting hotspots in prostate and lymph regions.

### III. Quantification

Table 6 identifies the values calculated by the software, displayed for each hotspot after selection by the user. The ITLV is a summative value and only displayed in the report. All calculations are variants of SUVs from PSMA PET/CT.

TABLE 6

Values Calculated by aPROMISE
Values reported for each selected hotspot and suspicious lesion:

| SUV-max | Represents the highest uptake in one voxel of the hotspot. |
|---|---|
| | $S U {V}_{m a x} = max \left(U p t a k e I n V o x e l ∈ l e s i o n v o l u m e\right)$ |
| SUV-mean | Calculated as the mean uptake of all voxels representing the hotspot. |
| | $n = v o x e l s ∈ l e s i o n v o l u m e , S U {V}_{m e a n} = {\sum }_{\begin{matrix}i ∈ l e s i o n \\ v o l u m e\end{matrix}} \frac{U p t a k e I n V o x e {l}_{i}}{n}$ |
| SUV-peak | Calculated as the mean of all voxels with a midpoint within 5 mm of the midpoint of the voxel where the SUV-max is located. |
| | $S U {V}_{p e a k} = {\sum }_{\begin{matrix}i : d i s t ( S U V m a x \\ p o i n t , i ) < 5 m m\end{matrix}} \frac{U p t a k e I n V o x e {l}_{i}}{n}$ |
| VOLUME | Calculated the number of voxels times the voxel volume, displayed in (ml). |
| | $\begin{matrix}V o x e l V o l u m e = \left(S p a c i n g . x ∗ S p a c i n g . y ∗ S p a c i n g . z\right) \left({\text{mm}}^{2}\right) \\ L e s i o n v o l u m e = V o x e l V o l u m e ∗ N b r O f V o x e l s\end{matrix}$ |
| LI | Lesion Index - Calculated based on the SUV reference values for aorta (also called blood pool) and liver. The Lesion Index is a real number between 0 and 3 based on the SUV-mean of the lesion in relation to a linear interpolation in the following spans: |
| | $\begin{matrix}0 \to S U {V}_{A o r t a} \to S U {V}_{L i v e r} \to 2 ∗ S U {V}_{L i v e r} \\ S U {V}_{m e a n} = S U {V}_{A o r t a} \to L I = 1 \\ S U {V}_{m e a n} = S U {V}_{L i v e r} \to L I = 2 \\ S U {V}_{m e a n} \geq 2 ∗ S U {V}_{L i v e r} \to L I = 3\end{matrix}$ |
| | If SUV references for either liver or aorta cannot be calculated, or if the aorta value is higher than the liver value, the Lesion Index will not be calculated and will be displayed as ‘-’. |
| Values reported for each lesion type at Patient Level: | |
| ILTV | Intensity-weighted Tissue Lesion Volume -For each lesion type the ITLV is calculated. An ITLV is the weighted sum of the lesion volumes for a specific type where the weight is the Lesion Index. |
| | $I T L V = {\sum }_{l e s i o n} L I ∗ \left(L e s i o n V o l u m e\right)$ |

### IV. Web-Based Platform Architecture

aPROMISE utilizes a microservice architecture. Deployment to AWS is handled in cloud formation scripts found in the AWS code repository. The aPROMISE cloud architecture is provided in FIG. 29A and the microservice communication design chart is provided in FIG. 29B.

### J. Imaging Agents

### I. PET Imaging Radionuclide Labelled PSMA Binding Agents

In certain embodiments, the radionuclide labelled PSMA binding agent is a radionuclide labelled PSMA binding agent appropriate for PET imaging.

In certain embodiments, the radionuclide labelled PSMA binding agent comprises [18F]DCFPyL (also referred to as PyL™; also referred to as DCFPyL-18F):

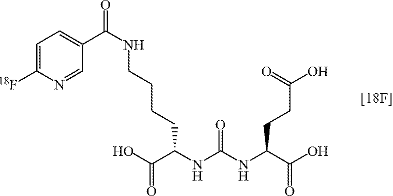 [SMILES: O=C(O)CC[C@H](NC(=O)N[C@@H](CCCCNC(=O)c1ccc([18F])nc1)C(=O)O)C(=O)O]

or a pharmaceutically acceptable salt thereof.

In certain embodiments, the radionuclide labelled PSMA binding agent comprises [18F]DCFBC:

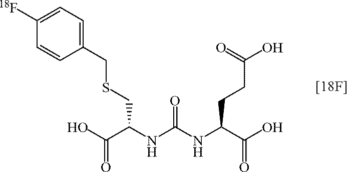 [SMILES: O=C(O)CC[C@H](NC(=O)N[C@@H](CSCc1ccc([18F])cc1)C(=O)O)C(=O)O]

or a pharmaceutically acceptable salt thereof.

In certain embodiments, the radionuclide labelled PSMA binding agent comprises ⁶⁸Ga-PSMA-HBED-CC (also referred to as ⁶⁸Ga-PSMA-11):

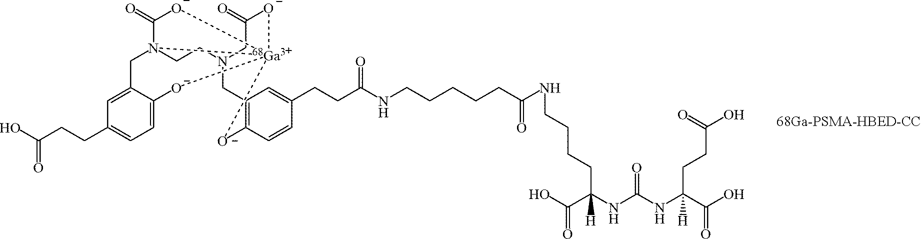

or a pharmaceutically acceptable salt thereof.

In certain embodiments, the radionuclide labelled PSMA binding agent comprises PSMA-617:

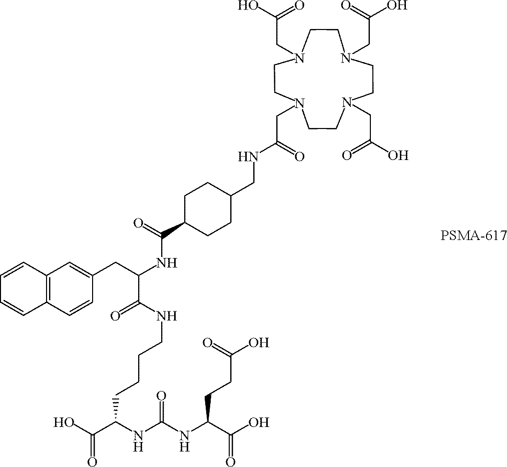

or a pharmaceutically acceptable salt thereof. In certain embodiments, the radionuclide labelled PSMA binding agent comprises ⁶⁸Ga-PSMA-617, which is PSMA-617 labelled with ⁶⁸Ga, or a pharmaceutically acceptable salt thereof. In certain embodiments, the radionuclide labelled PSMA binding agent comprises ¹⁷⁷Lu-PSMA-617, which is PSMA-617 labelled with ¹⁷⁷Lu, or a pharmaceutically acceptable salt thereof.

In certain embodiments, the radionuclide labelled PSMA binding agent comprises PSMA-I&T:

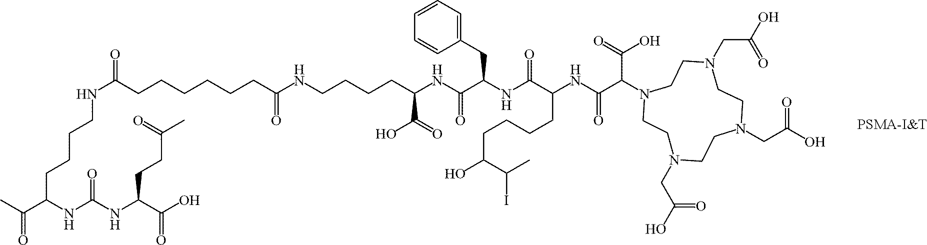 [SMILES: CC(=O)CC[C@H](NC(=O)NC(CCCCNC(=O)CCCCCCC(=O)NCCCC[C@@H](NC(=O)[C@@H](Cc1ccccc1)NC(=O)C(CCCCC(O)C(C)I)NC(=O)C(C(=O)O)N1CCN(CC(=O)O)CCN(CC(=O)O)CCN(CC(=O)O)CC1)C(=O)O)C(C)=O)C(=O)O]

or a pharmaceutically acceptable salt thereof. In certain embodiments, the radionuclide labelled PSMA binding agent comprises ⁶⁸Ga-PSMA-I&T, which is PSMA-I&T labelled with ⁶⁸Ga, or a pharmaceutically acceptable salt thereof.

In certain embodiments, the radionuclide labelled PSMA binding agent comprises PSMA-1007:

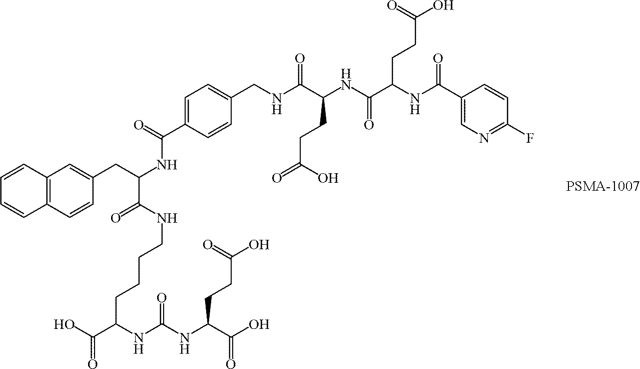 [SMILES: O=C(O)CCC(NC(=O)c1ccc(F)nc1)C(=O)N[C@@H](CCC(=O)O)C(=O)NCc1ccc(C(=O)NC(Cc2ccc3ccccc3c2)C(=O)NCCCCC(NC(=O)N[C@@H](CCC(=O)O)C(=O)O)C(=O)O)cc1]

or a pharmaceutically acceptable salt thereof. In certain embodiments, the radionuclide labelled PSMA binding agent comprises ¹⁸F-PSMA-1007, which is PSMA-1007 labelled with ¹⁸F, or a pharmaceutically acceptable salt thereof.

### II. SPECT Imaging Radionuclide Labelled PSMA Binding Agents

In certain embodiments, the radionuclide labelled PSMA binding agent is a radionuclide labelled PSMA binding agent appropriate for SPECT imaging.

In certain embodiments, the radionuclide labelled PSMA binding agent comprises 1404 (also referred to as MIP-1404):

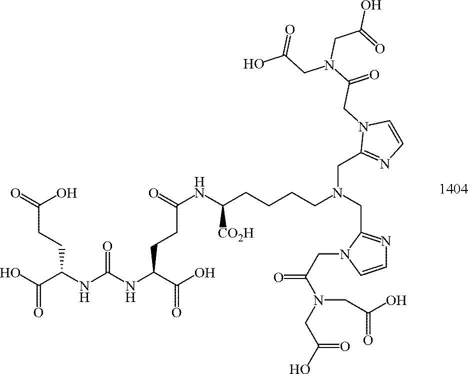 [SMILES: O=C(O)CC[C@H](NC(=O)N[C@@H](CCC(=O)N[C@@H](CCCCN(Cc1nccn1CC(=O)N(CC(=O)O)CC(=O)O)Cc1nccn1CC(=O)N(CC(=O)O)CC(=O)O)C(=O)O)C(=O)O)C(=O)O]

or a pharmaceutically acceptable salt thereof.

In certain embodiments, the radionuclide labelled PSMA binding agent comprises 1405 (also referred to as MIP-1405):

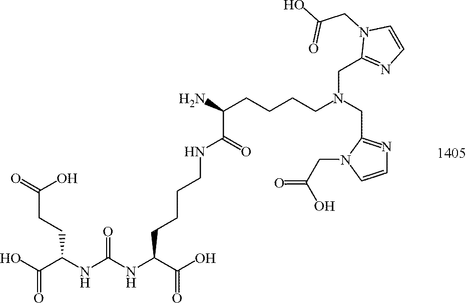 [SMILES: N[C@@H](CCCCN(Cc1nccn1CC(=O)O)Cc1nccn1CC(=O)O)C(=O)NCCCC[C@H](NC(=O)N[C@@H](CCC(=O)O)C(=O)O)C(=O)O]

or a pharmaceutically acceptable salt thereof.

In certain embodiments, the radionuclide labelled PSMA binding agent comprises 1427 (also referred to as MIP-1427):

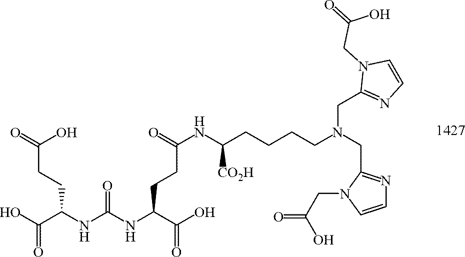 [SMILES: O=C(O)CC[C@H](NC(=O)N[C@@H](CCC(=O)N[C@@H](CCCCN(Cc1nccn1CC(=O)O)Cc1nccn1CC(=O)O)C(=O)O)C(=O)O)C(=O)O]

or a pharmaceutically acceptable salt thereof.

In certain embodiments, the radionuclide labelled PSMA binding agent comprises 1428 (also referred to as MIP-1428):

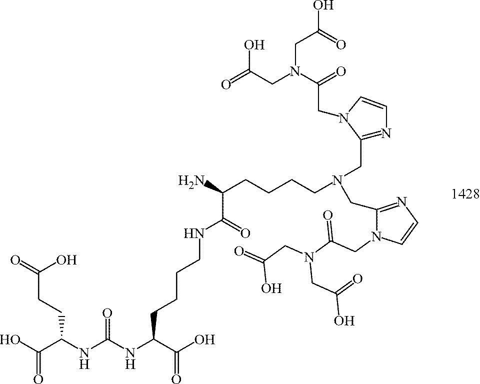 [SMILES: N[C@@H](CCCCN(Cc1nccn1CC(=O)N(CC(=O)O)CC(=O)O)Cc1nccn1CC(=O)N(CC(=O)O)CC(=O)O)C(=O)NCCCC[C@H](NC(=O)N[C@@H](CCC(=O)O)C(=O)O)C(=O)O]

or a pharmaceutically acceptable salt thereof.

In certain embodiments, the PSMA binding agent is labelled with a radionuclide by chelating it to a radioisotope of a metal [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)].

In certain embodiments, 1404 is labelled with a radionuclide (e.g., chelated to a radioisotope of a metal). In certain embodiments, the radionuclide labelled PSMA binding agent comprises ^{99m}Tc-MIP-1404, which is 1404 labelled with (e.g., chelated to) ^{99m}Tc:

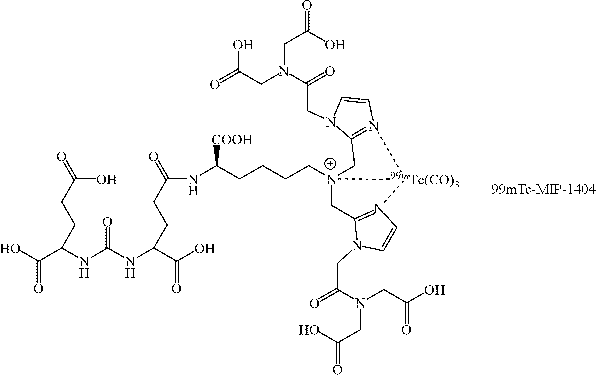

or a pharmaceutically acceptable salt thereof. In certain embodiments, 1404 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1404, with the other metal radioisotope substituted for ^{99m}Tc.

In certain embodiments, 1405 is labelled with a radionuclide (e.g., chelated to a radioisotope of a metal). In certain embodiments, the radionuclide labelled PSMA binding agent comprises ^{99m}Tc-MIP-1405, which is 1405 labelled with (e.g., chelated to) ^{99m}Tc:

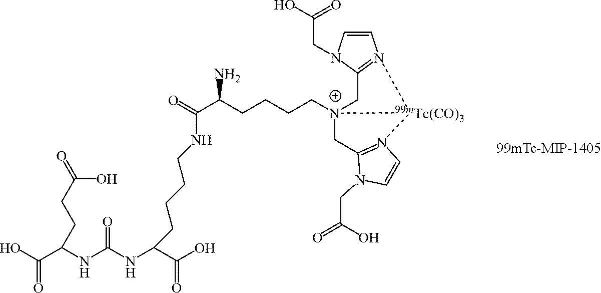

or a pharmaceutically acceptable salt thereof. In certain embodiments, 1405 may be chelated to other metal radioisotopes [e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] to form a compound having a structure similar to the structure shown above for ^{99m}Tc-MIP-1405, with the other metal radioisotope substituted for ^{99m}Tc.

In certain embodiments, 1427 is labelled with (e.g., chelated to) a radioisotope of a metal, to form a compound according to the formula below:

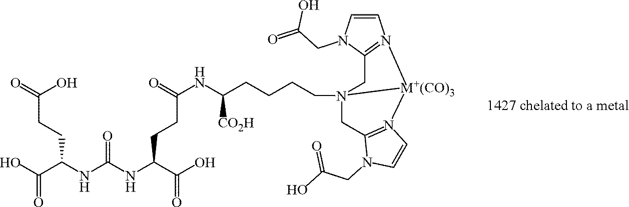

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹in); e.g., a radioisotope of copper (Cu) (e.g., ⁶⁷Cu)] with which 1427 is labelled.

In certain embodiments, 1428 is labelled with (e.g., chelated to) a radioisotope of a metal, to form a compound according to the formula below:

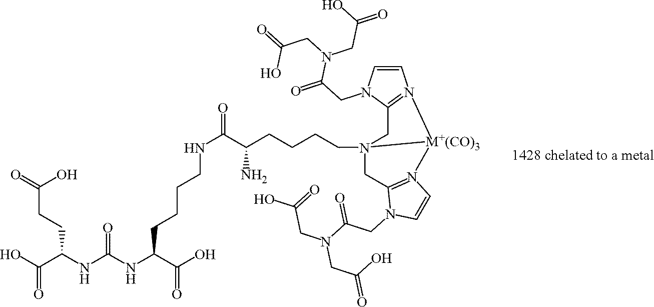

or a pharmaceutically acceptable salt thereof, wherein M is a metal radioisotope [e.g., a radioisotope of technetium (Tc) (e.g., technetium-99m (^{99m}Tc)); e.g., a radioisotope of rhenium (Re) (e.g., rhenium-188 (¹⁸⁸Re); e.g., rhenium-186 (¹⁸⁶Re)); e.g., a radioisotope of yttrium (Y) (e.g., ⁹⁰Y); e.g., a radioisotope of lutetium (Lu)(e.g., ¹⁷⁷Lu); e.g., a radioisotope of gallium (Ga) (e.g., ⁶⁸Ga; e.g., ⁶⁷Ga); e.g., a radioisotope of indium (e.g., ¹¹¹In); e.g., a radioisotope of copper (Cu) (e.g., ⁶⁷Cu)] with which 1428 is labelled.

In certain embodiments, the radionuclide labelled PSMA binding agent comprises PSMA I&S:

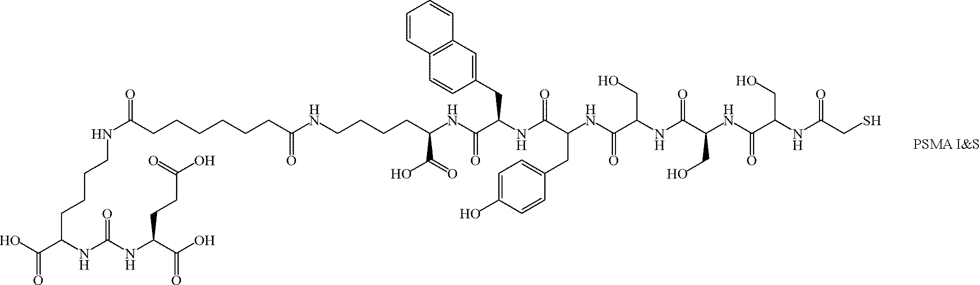 [SMILES: O=C(O)CC[C@H](NC(=O)NC(CCCCNC(=O)CCCCCCC(=O)NCCCC[C@@H](NC(=O)[C@@H](Cc1ccc2ccccc2c1)NC(=O)C(Cc1ccc(O)cc1)NC(=O)C(CO)NC(=O)[C@H](CO)NC(=O)C(CO)NC(=O)CS)C(=O)O)C(=O)O)C(=O)O]

or a pharmaceutically acceptable salt thereof. In certain embodiments, the radionuclide labelled PSMA binding agent comprises ^{99m}Tc-PSMA I&S, which is PSMA I&S labelled with ^{99m}Tc, or a pharmaceutically acceptable salt thereof.

### K. Computer System and Network Architecture

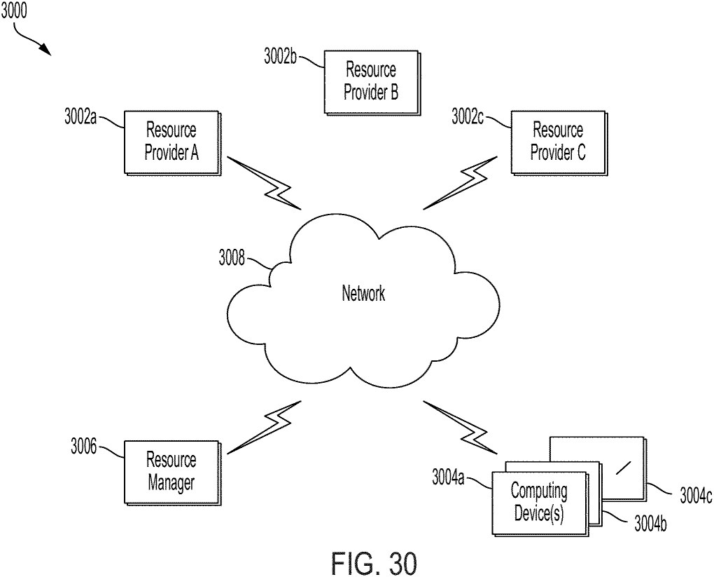

As shown in FIG. 30, an implementation of a network environment 3000 for use in providing systems, methods, and architectures described herein is shown and described. In brief overview, referring now to FIG. 30, a block diagram of an exemplary cloud computing environment 3000 is shown and described. The cloud computing environment 3000 may include one or more resource providers 3002a, 3002b, 3002c (collectively, 3002). Each resource provider 3002 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 3002 may be connected to any other resource provider 3002 in the cloud computing environment 3000. In some implementations, the resource providers 3002 may be connected over a computer network 3008. Each resource provider 3002 may be connected to one or more computing device 3004a, 3004b, 3004c (collectively, 3004), over the computer network 3008.

The cloud computing environment 3000 may include a resource manager 3006. The resource manager 3006 may be connected to the resource providers 3002 and the computing devices 3004 over the computer network 3008. In some implementations, the resource manager 3006 may facilitate the provision of computing resources by one or more resource providers 3002 to one or more computing devices 3004. The resource manager 3006 may receive a request for a computing resource from a particular computing device 3004. The resource manager 3006 may identify one or more resource providers 3002 capable of providing the computing resource requested by the computing device 3004. The resource manager 3006 may select a resource provider 3002 to provide the computing resource. The resource manager 3006 may facilitate a connection between the resource provider 3002 and a particular computing device 3004. In some implementations, the resource manager 3006 may establish a connection between a particular resource provider 3002 and a particular computing device 3004. In some implementations, the resource manager 3006 may redirect a particular computing device 3004 to a particular resource provider 3002 with the requested computing resource.

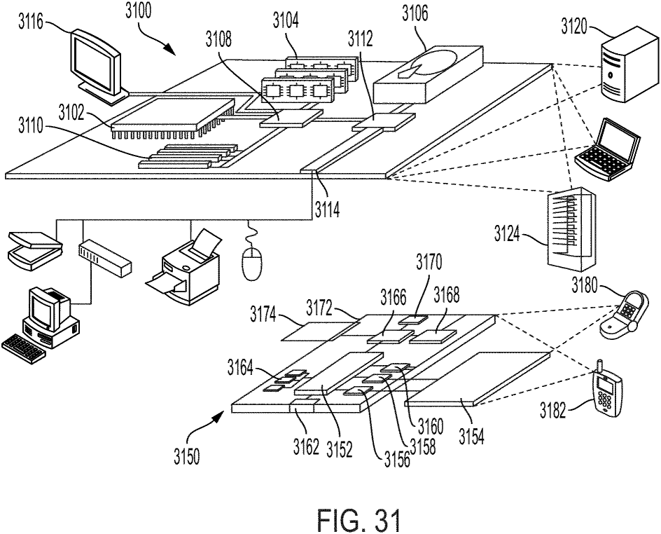

FIG. 31 shows an example of a computing device 3100 and a mobile computing device 3150 that can be used to implement the techniques described in this disclosure. The computing device 3100 is intended to represent various forms of digital computers, such as laptops, desktops, workstations, personal digital assistants, servers, blade servers, mainframes, and other appropriate computers. The mobile computing device 3150 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 3100 includes a processor 3102, a memory 3104, a storage device 3106, a high-speed interface 3108 connecting to the memory 3104 and multiple high-speed expansion ports 3110, and a low-speed interface 3112 connecting to a low-speed expansion port 3114 and the storage device 3106. Each of the processor 3102, the memory 3104, the storage device 3106, the high-speed interface 3108, the high-speed expansion ports 3110, and the low-speed interface 3112, are interconnected using various busses, and may be mounted on a common motherboard or in other manners as appropriate. The processor 3102 can process instructions for execution within the computing device 3100, including instructions stored in the memory 3104 or on the storage device 3106 to display graphical information for a GUI on an external input/output device, such as a display 3116 coupled to the high-speed interface 3108. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 3104 stores information within the computing device 3100. In some implementations, the memory 3104 is a volatile memory unit or units. In some implementations, the memory 3104 is a non-volatile memory unit or units. The memory 3104 may also be another form of computer-readable medium, such as a magnetic or optical disk.

The storage device 3106 is capable of providing mass storage for the computing device 3100. In some implementations, the storage device 3106 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 3102), perform one or more methods, such as those described above. The instructions can also be stored by one or more storage devices such as computer- or machine-readable mediums (for example, the memory 3104, the storage device 3106, or memory on the processor 3102).

The high-speed interface 3108 manages bandwidth-intensive operations for the computing device 3100, while the low-speed interface 3112 manages lower bandwidth-intensive operations. Such allocation of functions is an example only. In some implementations, the high-speed interface 3108 is coupled to the memory 3104, the display 3116 (e.g., through a graphics processor or accelerator), and to the high-speed expansion ports 3110, which may accept various expansion cards (not shown). In the implementation, the low-speed interface 3112 is coupled to the storage device 3106 and the low-speed expansion port 3114. The low-speed expansion port 3114, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 3100 may be implemented in a number of different forms, as shown in the figure. For example, it may be implemented as a standard server 3120, or multiple times in a group of such servers. In addition, it may be implemented in a personal computer such as a laptop computer 3122. It may also be implemented as part of a rack server system 3124. Alternatively, components from the computing device 3100 may be combined with other components in a mobile device (not shown), such as a mobile computing device 3150. Each of such devices may contain one or more of the computing device 3100 and the mobile computing device 3150, and an entire system may be made up of multiple computing devices communicating with each other.

The mobile computing device 3150 includes a processor 3152, a memory 3164, an input/output device such as a display 3154, a communication interface 3166, and a transceiver 3168, among other components. The mobile computing device 3150 may also be provided with a storage device, such as a micro-drive or other device, to provide additional storage. Each of the processor 3152, the memory 3164, the display 3154, the communication interface 3166, and the transceiver 3168, are interconnected using various buses, and several of the components may be mounted on a common motherboard or in other manners as appropriate.

The processor 3152 can execute instructions within the mobile computing device 3150, including instructions stored in the memory 3164. The processor 3152 may be implemented as a chipset of chips that include separate and multiple analog and digital processors. The processor 3152 may provide, for example, for coordination of the other components of the mobile computing device 3150, such as control of user interfaces, applications run by the mobile computing device 3150, and wireless communication by the mobile computing device 3150.

The processor 3152 may communicate with a user through a control interface 3158 and a display interface 3156 coupled to the display 3154. The display 3154 may be, for example, a TFT (Thin-Film-Transistor Liquid Crystal Display) display or an OLED (Organic Light Emitting Diode) display, or other appropriate display technology. The display interface 3156 may comprise appropriate circuitry for driving the display 3154 to present graphical and other information to a user. The control interface 3158 may receive commands from a user and convert them for submission to the processor 3152. In addition, an external interface 3162 may provide communication with the processor 3152, so as to enable near area communication of the mobile computing device 3150 with other devices. The external interface 3162 may provide, for example, for wired communication in some implementations, or for wireless communication in other implementations, and multiple interfaces may also be used.

The memory 3164 stores information within the mobile computing device 3150. The memory 3164 can be implemented as one or more of a computer-readable medium or media, a volatile memory unit or units, or a non-volatile memory unit or units. An expansion memory 3174 may also be provided and connected to the mobile computing device 3150 through an expansion interface 3172, which may include, for example, a SIMM (Single In Line Memory Module) card interface. The expansion memory 3174 may provide extra storage space for the mobile computing device 3150, or may also store applications or other information for the mobile computing device 3150. Specifically, the expansion memory 3174 may include instructions to carry out or supplement the processes described above, and may include secure information also. Thus, for example, the expansion memory 3174 may be provide as a security module for the mobile computing device 3150, and may be programmed with instructions that permit secure use of the mobile computing device 3150. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier that the instructions, when executed by one or more processing devices (for example, processor 3152), perform one or more methods, such as those described above. The instructions can also be stored by one or more storage devices, such as one or more computer- or machine-readable mediums (for example, the memory 3164, the expansion memory 3174, or memory on the processor 3152). In some implementations, the instructions can be received in a propagated signal, for example, over the transceiver 3168 or the external interface 3162.

The mobile computing device 3150 may communicate wirelessly through the communication interface 3166, which may include digital signal processing circuitry where necessary. The communication interface 3166 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 3168 using a radio-frequency. In addition, short-range communication may occur, such as using a Bluetooth®, Wi-Fi™, or other such transceiver (not shown). In addition, a GPS (Global Positioning System) receiver module 3170 may provide additional navigation- and location-related wireless data to the mobile computing device 3150, which may be used as appropriate by applications running on the mobile computing device 3150.

The mobile computing device 3150 may also communicate audibly using an audio codec 3160, which may receive spoken information from a user and convert it to usable digital information. The audio codec 3160 may likewise generate audible sound for a user, such as through a speaker, e.g., in a handset of the mobile computing device 3150. Such sound may include sound from voice telephone calls, may include recorded sound (e.g., voice messages, music files, etc.) and may also include sound generated by applications operating on the mobile computing device 3150.

The mobile computing device 3150 may be implemented in a number of different forms, as shown in the figure. For example, it may be implemented as a cellular telephone 3180. It may also be implemented as part of a smart-phone 3182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the various modules described herein can be separated, combined or incorporated into single or combined modules. The modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

The various described embodiments of the invention may be used in conjunction with one or more other embodiments unless technically incompatible. It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) automatically detecting, by the processor, using a machine learning module, one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image; and
(c) storing and/or providing, for display and/or further processing, the hotspot list and/or the 3D hotspot map.

2. The method of claim 1, wherein the machine learning module receives, as input, at least a portion of the 3D functional image and automatically detects the one or more hotspots based at least in part on intensities of voxels of the received portion of the 3D functional image.

3. The method of claim 1 or 2, wherein the machine learning module receives, as input, a 3D segmentation map that identifies one or more volumes of interest (VOIs) within the 3D functional image, each VOI corresponding to a particular target tissue region and/or a particular anatomical region within the subject.

4. The method of any one of the preceding claims,
comprising receiving, by the processor, a 3D anatomical image of the subject obtained using an anatomical imaging modality, wherein the 3D anatomical image comprises a graphical representation of tissue within the subject,
and wherein the machine learning module receives at least two channels of input, said input channels comprising a first input channel corresponding to at least a portion of the 3D anatomical image and a second input channel corresponding to at least a portion of the 3D functional image.

5. The method of claim 4, wherein the machine learning module receives, as input, a 3D segmentation map that identifies, within the 3D functional image and/or the 3D anatomical image, one or more volumes of interest (VOIs), each VOI corresponding to a particular target tissue region and/or a particular anatomical region.

6. The method of claim 5, comprising automatically segmenting, by the processor, the 3D anatomical image, thereby creating the 3D segmentation map.

7. The method of any one of the preceding claims, wherein the machine learning module is a region-specific machine learning module that receives, as input, a specific portion of the 3D functional image corresponding to one or more specific tissue regions and/or anatomical regions of the subject.

8. The method of any one of the preceding claims, wherein the machine learning module generates, as output, the hotspot list.

9. The method of any one of the preceding claims, wherein the machine learning module generates, as output, the 3D hotspot map.

10. The method of any one of the preceding claims, comprising:
(d) determining, by the processor, for each hotspot of at least a portion of the hotspots, a lesion likelihood classification corresponding to a likelihood of the hotspot representing a lesion within the subject.

11. The method of claim 10, wherein step (d) comprises using the machine learning module to determine, for each hotspot of the portion, the lesion likelihood classification.

12. The method of claim 10, wherein step (d) comprises using a second machine learning module to determine the lesion likelihood classification for each hotspot.

13. The method of claim 12, comprising determining, by the processor, for each hotspot, a set of one or more hotspot features and using the set of the one or more hotspot features as input to the second machine learning module.

14. The method of any one of claims 10 to 13, comprising:
(e) selecting, by the processor, based at least in part on the lesion likelihood classifications for the hotspots, a subset of the one or more hotspots corresponding to hotspots having a high likelihood of corresponding to cancerous lesions.

15. The method of any one of the preceding claims, comprising:
(f) adjusting intensities of voxels of the 3D functional image, by the processor, to correct for intensity bleed from one or more high-intensity volumes of the 3D functional image, each of the one or more high-intensity volumes corresponding to a high-uptake tissue region within the subject associated with high radiopharmaceutical uptake under normal circumstances.

16. The method of claim 15, wherein step (f) comprises correcting for intensity bleed from a plurality of high-intensity volumes one at a time, in a sequential fashion.

17. The method of claims 15 or 16, wherein the one or more high-intensity volumes correspond to one or more high-uptake tissue regions selected from the group consisting of a kidney, a liver, and a bladder.

18. The method of any one of the preceding claims, comprising:
(g) determining, by the processor, for each of at least a portion of the one or more hotspots, a corresponding lesion index indicative of a level of radiopharmaceutical uptake within and/or size of an underlying lesion to which the hotspot corresponds.

19. The method of claim 18, wherein step (g) comprises comparing an intensity (intensities) of one or more voxels associated with the hotspot with one or more reference values, each reference value associated with a particular reference tissue region of a reference volume corresponding to the reference tissue region.

20. The method of claim 19, wherein the one or more reference values comprise one or more members selected from the group consisting of an aorta reference value associated with an aorta portion of the subject and a liver reference value associated with a liver of the subject.

21. The method of claim 19 or 20, wherein, for at least one particular reference value associated with a particular reference tissue region, determining the particular reference value comprises fitting intensities of voxels within a particular reference volume corresponding to the particular reference tissue region to a multi-component mixture model.

22. The method of any one of claims 18 to 21, comprising using the determined lesion index values compute an overall risk index for the subject, indicative of a caner status and/or risk for the subject.

23. The method of any one of the preceding claims, comprising determining, by the processor, for each hotspot, an anatomical classification corresponding to a particular anatomical region and/or group of anatomical regions within the subject in which the potential cancerous lesion that the hotspot represents is determined to be located.

24. The method of any one of the preceding claims, comprising:
(h) causing, by the processor, for display within a graphical user interface (GUI), rendering of a graphical representation of at least a portion of the one or more hotspots for review by a user.

25. The method of claim 24, comprising:
(i) receiving, by the processor, via the GUI, a user selection of a subset of the one or more hotspots confirmed via user review as likely to represent underlying cancerous lesions within the subject.

26. The method of any one of the preceding claims, wherein the 3D functional image comprises a PET or SPECT image obtained following administration of an agent to the subject.

27. The method of claim 26, wherein the agent comprises a PSMA binding agent.

28. The method of claim 26 or 27, wherein the agent comprises ¹⁸F.

29. The method of claim 27 or 28, wherein the agent comprises [18F]DCFPyL.

30. The method of claim 27 or 28, wherein the agent comprises PSMA-11.

31. The method of claim 26 or 27, wherein the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

32. The method of any one of the preceding claims, wherein the machine learning module implements a neural network.

33. The method of any one of the preceding claims, wherein the processor is a processor of a cloud-based system.

34. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) receiving, by the processor, a 3D anatomical image of the subject obtained using an anatomical imaging modality, wherein the 3D anatomical image comprises a graphical representation of tissue within the subject;
(c) automatically detecting, by the processor, using a machine learning module, one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image,
wherein the machine learning module receives at least two channels of input, said input channels comprising a first input channel corresponding to at least a portion of the 3D anatomical image and a second input channel corresponding to at least a portion of the 3D functional image and/or anatomical information derived therefrom; and
(d) storing and/or providing, for display and/or further processing, the hotspot list and/or the 3D hotspot map.

35. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) automatically detecting, by the processor, using a first machine learning module, one or more hotspots within the 3D functional image, each hotspot corresponding to a local region of elevated intensity with respect to its surrounding and representing a potential cancerous lesion within the subject, thereby creating a hotspot list identifying, for each hotspot, a location of the hotspot;
(c) automatically determining, by the processor, using a second machine learning module and the hotspot list, for each of the one or more hotspots, a corresponding 3D hotspot volume within the 3D functional image, thereby creating a 3D hotspot map; and
(d) storing and/or providing, for display and/or further processing, the hotspot list and/or the 3D hotspot map.

36. The method of claim 35, comprising:
(e) determining, by the processor, for each hotspot of at least a portion of the hotspots, a lesion likelihood classification corresponding to a likelihood of the hotspot representing a lesion within the subject.

37. The method of claim 36, wherein step (e) comprises using a third machine learning module to determine the lesion likelihood classification for each hotspot.

38. The method of any one of claims 35 to 37, comprising:
(f) selecting, by the processor, based at least in part on the lesion likelihood classifications for the hotspots, a subset of the one or more hotspots corresponding to hotspots having a high likelihood of corresponding to cancerous lesions.

39. A method of measuring intensity values within a reference volume corresponding to a reference tissue region so as to avoid impact from tissue regions associated with low radiopharmaceutical uptake, the method comprising:
(a) receiving, by a processor of a computing device, a 3D functional image of a subject, said 3D functional image obtained using a functional imaging modality;
(b) identifying, by the processor, the reference volume within the 3D functional image;
(c) fitting, by the processor, a multi-component mixture model to intensities of voxels within the reference volume;
(d) identifying, by the processor, a major mode of the multi-component model;
(e) determining, by the processor, a measure of intensities corresponding to the major mode, thereby determining a reference intensity value corresponding to a measure of intensity of voxels that are (i) within the reference tissue volume and (ii) associated with the major mode;
(f) detecting, by the processor, within the functional image, one or more hotspots corresponding potential cancerous lesions; and
(g) determining, by the processes or, for each hotspot of at least a portion of the detected hotspots, a lesion index value, using at least the reference intensity value.

40. A method of correcting for intensity bleed from due to high-uptake tissue regions within the subject that are associated with high radiopharmaceutical uptake under normal circumstances, the method comprising:
(a) receiving, by a processor of a computing device, a 3D functional image of the subject, said 3D functional image obtained using a functional imaging modality;
(b) identifying, by the processor, a high-intensity volume within the 3D functional image, said high intensity volume corresponding to a particular high-uptake tissue region in which high radiopharmaceutical uptake occurs under normal circumstances;
(c) identifying, by the processor, based on the identified high-intensity volume, a suppression volume within the 3D functional image, said suppression volume corresponding to a volume lying outside and within a predetermined decay distance from a boundary of the identified high intensity volume;
(d) determining, by the processor, a background image corresponding to the 3D functional image with intensities of voxels within the high-intensity volume replaced with interpolated values determined based on intensities of voxels of the 3D functional image within the suppression volume;
(e) determining, by the processor, an estimation image by subtracting intensities of voxels of the background image from intensities of voxels from the 3D functional image;
(f) determining, by the processor, a suppression map by:
extrapolating intensities of voxels of the estimation image corresponding to the high-intensity volume to locations of voxels within the suppression volume to determine intensities of voxels of the suppression map corresponding to the suppression volume; and
setting intensities of voxels of the suppression map corresponding to locations outside the suppression volume to zero; and
(g) adjusting, by the processor, intensities of voxels of the 3D functional image based on the suppression map, thereby correcting for intensity bleed from the high-intensity volume.

41. The method of claim 40, comprising performing steps (b) through (g) for each of a plurality of high-intensity volumes in a sequential manner, thereby correcting for intensity bleed from each of the plurality of high-intensity volumes.

42. The method of claim 41, wherein the plurality of high-intensity volumes comprise one or more members selected from the group consisting of a kidney, a liver, and a bladder.

43. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) automatically detecting, by the processor, one or more hotspots within the 3D functional image, each hotspot corresponding to a local region of elevated intensity with respect to its surrounding and representing a potential cancerous lesion within the subject;
(c) causing, by the processor, rendering of a graphical representation of the one or more hotspots for display within an interactive graphical user interface (GUI);
(d) receiving, by the processor, via the interactive GUI, a user selection of a final hotspot set comprising at least a portion of the one or more automatically detected hotspots; and
(e) storing and/or providing, for display and/or further processing, the final hotspot set.

44. The method of claim 43, comprising:
(f) receiving, by the processor, via the GUI, a user selection of one or more additional, user-identified, hotspots for inclusion in the final hotspot set; and
(g) updating, by the processor, the final hotspot set to include the one or more additional user-identified hotspots.

45. The method of either claim 43 or 44, wherein step (b) comprises using one or more machine learning modules.

46. A method for automatically processing 3D images of a subject to identify and characterize cancerous lesions within the subject, the method comprising:
(a) receiving, by a processor of a computing device, a 3D functional image of the subject obtained using a functional imaging modality;
(b) automatically detecting, by the processor, one or more hotspots within the 3D functional image, each hotspot corresponding to a local region of elevated intensity with respect to its surrounding and representing a potential cancerous lesion within the subject;
(c) automatically determining, by the processor, for each of at least a portion of the one or more hotspots, an anatomical classification corresponding to a particular anatomical region and/or group of anatomical regions within the subject in which the potential cancerous lesion that the hotspot represents is determined to be located; and
(d) storing and/or providing, for display and/or further processing, an identification of the one or more hotspots along with, for each hotspot, the anatomical classification corresponding to the hotspot.

47. The method of claim 46, wherein step (b) comprises using one or more machine learning modules.

48. A system for automatically processing 3D images of a subject to identify and/or characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) automatically detect, using a machine learning module, one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image; and
(c) store and/or provide, for display and/or further processing, the hotspot list and/or the 3D hotspot map.

49. The system of claim 48, wherein the machine learning module receives, as input, at least a portion of the 3D functional image and automatically detects the one or more hotspots based at least in part on intensities of voxels of the received portion of the 3D functional image.

50. The system of claim 48 or 49, wherein the machine learning module receives, as input, a 3D segmentation map that identifies one or more volumes of interest (VOIs) within the 3D functional image, each VOI corresponding to a particular target tissue region and/or a particular anatomical region within the subject.

51. The system of any one of claims 48 to 50, wherein the instructions cause the processor to:
receive a 3D anatomical image of the subject obtained using an anatomical imaging modality, wherein the 3D anatomical image comprises a graphical representation of tissue within the subject,
and wherein the machine learning module receives at least two channels of input, said input channels comprising a first input channel corresponding to at least a portion of the 3D anatomical image and a second input channel corresponding to at least a portion of the 3D functional image.

52. The system of claim 51, wherein the machine learning module receives, as input, a 3D segmentation map that identifies, within the 3D functional image and/or the 3D anatomical image, one or more volumes of interest (VOIs), each VOI corresponding to a particular target tissue region and/or a particular anatomical region.

53. The system of claim 52, wherein the instructions cause the processor to automatically segment the 3D anatomical image, thereby creating the 3D segmentation map.

54. The system of any one of claims 48 to 53, wherein the machine learning module is a region-specific machine learning module that receives, as input, a specific portion of the 3D functional image corresponding to one or more specific tissue regions and/or anatomical regions of the subject.

55. The system of any one of claims 48 to 54, wherein the machine learning module generates, as output, the hotspot list.

56. The system of any one of claims 48 to 55, wherein the machine learning module generates, as output, the 3D hotspot map.

57. The system of any one of claims 48 to 56, wherein the instructions cause the processor to:
(d) determine, for each hotspot of at least a portion of the hotspots, a lesion likelihood classification corresponding to a likelihood of the hotspot representing a lesion within the subject.

58. The system of claim 57, wherein at step (d) the instructions cause the processor to use the machine learning module to determine, for each hotspot of the portion, the lesion likelihood classification.

59. The system of claim 57, wherein at step (d) the instructions cause the processor to use a second machine learning module to determine the lesion likelihood classification for each hotspot.

60. The method of claim 59, wherein the instructions cause the processor to determine, for each hotspot, a set of one or more hotspot features and using the set of the one or more hotspot features as input to the second machine learning module.

61. The system of any one of claims 57 to 60, wherein the instructions cause the processor to:
(e) select, based at least in part on the lesion likelihood classifications for the hotspots, a subset of the one or more hotspots corresponding to hotspots having a high likelihood of corresponding to cancerous lesions.

62. The system of any one of claims 48 to 61, wherein the instructions cause the processor to:
(f) adjust intensities of voxels of the 3D functional image, by the processor, to correct for intensity bleed from one or more high-intensity volumes of the 3D functional image, each of the one or more high-intensity volumes corresponding to a high-uptake tissue region within the subject associated with high radiopharmaceutical uptake under normal circumstances.

63. The system of claim 62, wherein at step (f) the instructions cause the processor to correct for intensity bleed from a plurality of high-intensity volumes one at a time, in a sequential fashion.

64. The system of claim 62 or 63 wherein the one or more high-intensity volumes correspond to one or more high-uptake tissue regions selected from the group consisting of a kidney, a liver, and a bladder.

65. The system of any one of claims 48 to 64, wherein the instructions cause the processor to:
(g) determine, for each of at least a portion of the one or more hotspots, a corresponding lesion index indicative of a level of radiopharmaceutical uptake within and/or size of an underlying lesion to which the hotspot corresponds.

66. The system of claim 65, wherein at step (g) the instructions cause the processor to compare an intensity (intensities) of one or more voxels associated with the hotspot with one or more reference values, each reference value associated with a particular reference tissue region within the subject and determined based on intensities of a reference volume corresponding to the reference tissue region.

67. The system of claim 66, wherein the one or more reference values comprise one or more members selected from the group consisting of an aorta reference value associated with an aorta portion of the subject and a liver reference value associated with a liver of the subject.

68. The system of claim 66 or 67, wherein, for at least one particular reference value associated with a particular reference tissue region, the instructions cause the processor to determine the particular reference value by fitting intensities of voxels within a particular reference volume corresponding to the particular reference tissue region to a multi-component mixture model.

69. The system of any one of claims 65 to 68, wherein the instructions cause the processor to use the determined lesion index values compute an overall risk index for the subject, indicative of a caner status and/or risk for the subject.

70. The system of any one of claims 48 to 69, wherein the instructions cause the processor to determine, for each hotspot, an anatomical classification corresponding to a particular anatomical region and/or group of anatomical regions within the subject in which the potential cancerous lesion that the hotspot represents is determined to be located.

71. The system of any one of claims 48 to 70, wherein the instructions cause the processor to:
(h) causing, for display within a graphical user interface (GUI), rendering of a graphical representation of at least a portion of the one or more hotspots for review by a user.

72. The system of claim 71, wherein the instructions cause the processor to:
(i) receiving, via the GUI, a user selection of a subset of the one or more hotspots confirmed via user review as likely to represent underlying cancerous lesions within the subject.

73. The system of any one of claims 48 to 72, wherein the 3D functional image comprises a PET or SPECT image obtained following administration of an agent to the subject.

74. The system of claim 73, wherein the agent comprises a PSMA binding agent.

75. The system of claim 73 or 74, wherein the agent comprises ¹⁸F.

76. The system of claim 74, wherein the agent comprises [18F]DCFPyL.

77. The system of claim 74 or 75, wherein the agent comprises PSMA-11.

78. The system of claim 73 or 74, wherein the agent comprises one or more members selected from the group consisting of ^{99m}Tc, ⁶⁸Ga, ¹⁷⁷Lu, ²²⁵Ac, ¹¹¹In, ¹²³I, ¹²⁴I, and ¹³¹I.

79. The system of any one of claims 48 to 78, wherein the machine learning module implements a neural network.

80. The system of any one of claims 48 to 79, wherein the processor is a processor of a cloud-based system.

81. A system for automatically processing 3D images of a subject to identify and/or characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) receive a 3D anatomical image of the subject obtained using an anatomical imaging modality, wherein the 3D anatomical image comprises a graphical representation of tissue within the subject;
(c) automatically detect, using a machine learning module, one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image,
wherein the machine learning module receives at least two channels of input, said input channels comprising a first input channel corresponding to at least a portion of the 3D anatomical image and a second input channel corresponding to at least a portion of the 3D functional image and/or anatomical information derived therefrom; and
(d) store and/or provide, for display and/or further processing, the hotspot list and/or the 3D hotspot map.

82. A system for automatically processing 3D images of a subject to identify and/or characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) automatically detect, using a first machine learning module, one or more hotspots within the 3D functional image, each hotspot corresponding to a local region of elevated intensity with respect to its surrounding and representing a potential cancerous lesion within the subject, thereby creating a hotspot list identifying, for each hotspot, a location of the hotspot;
(c) automatically determine, using a second machine learning module and the hotspot list, for each of the one or more hotspots, a corresponding 3D hotspot volume within the 3D functional image, thereby creating a 3D hotspot map; and
(d) store and/or provide, for display and/or further processing, the hotspot list and/or the 3D hotspot map.

83. The system of claim 82, wherein the instructions cause the processor to:
(e) determine, for each hotspot of at least a portion of the hotspots, a lesion likelihood classification corresponding to a likelihood of the hotspot representing a lesion within the subject.

84. The system of claim 83, wherein at step (e) the instructions cause the processor to use a third machine learning module to determine the lesion likelihood classification for each hotspot.

85. The system of any one of claims 82 to 84, wherein the instructions cause the processor to:
(f) select, based at least in part on the lesion likelihood classifications for the hotspots, a subset of the one or more hotspots corresponding to hotspots having a high likelihood of corresponding to cancerous lesions.

86. A system for measuring intensity values within a reference volume corresponding to a reference tissue region so as to avoid impact from tissue regions associated with low radiopharmaceutical uptake, the system comprising:
a processor of a computing device; and
a memory having instructions stored thereon, wherein the instructions, when executed by the processor, cause the processor to:
(a) receive a 3D functional image of a subject, said 3D functional image obtained using a functional imaging modality;
(b) identify the reference volume within the 3D functional image;
(c) fit a multi-component mixture model to intensities of voxels within the reference volume;
(d) identify a major mode of the multi-component model;
(e) determine a measure of intensities corresponding to the major mode, thereby determining a reference intensity value corresponding to a measure of intensity of voxels that are (i) within the reference tissue volume and (ii) associated with the major mode;
(f) detect, within the 3D functional image, one or more hotspots corresponding potential cancerous lesions; and
(g) determine, for each hotspot of at least a portion of the detected hotspots, a lesion index value, using at least the reference intensity value.

87. A system for correcting for intensity bleed from due to high-uptake tissue regions within the subject that are associated with high radiopharmaceutical uptake under normal circumstances, the method comprising:
(a) receive a 3D functional image of the subject, said 3D functional image obtained using a functional imaging modality;
(b) identify a high-intensity volume within the 3D functional image, said high intensity volume corresponding to a particular high-uptake tissue region in which high radiopharmaceutical uptake occurs under normal circumstances;
(c) identify, based on the identified high-intensity volume, a suppression volume within the 3D functional image, said suppression volume corresponding to a volume lying outside and within a predetermined decay distance from a boundary of the identified high intensity volume;
(d) determine a background image corresponding to the 3D functional image with intensities of voxels within the high-intensity volume replaced with interpolated values determined based on intensities of voxels of the 3D functional image within the suppression volume;
(e) determine an estimation image by subtracting intensities of voxels of the background image from intensities of voxels from the 3D functional image;
(f) determine a suppression map by:
extrapolating intensities of voxels of the estimation image corresponding to the high-intensity volume to locations of voxels within the suppression volume to determine intensities of voxels of the suppression map corresponding to the suppression volume; and
setting intensities of voxels of the suppression map corresponding to locations outside the suppression volume to zero; and
(g) adjust intensities of voxels of the 3D functional image based on the suppression map, thereby correcting for intensity bleed from the high-intensity volume.

88. The system of claim 87, wherein the instructions cause the processor to perform steps (b) through (g) for each of a plurality of high-intensity volumes in a sequential manner, thereby correcting for intensity bleed from each of the plurality of high-intensity volumes.

89. The system of claim 88, wherein the plurality of high-intensity volumes comprise one or more members selected from the group consisting of a kidney, a liver, and a bladder.

90. A system for automatically processing 3D images of a subject to identify and/or characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) automatically detect one or more hotspots within the 3D functional image, each hotspot corresponding to a local region of elevated intensity with respect to its surrounding and representing a potential cancerous lesion within the subject;
(c) cause rendering of a graphical representation of the one or more hotspots for display within an interactive graphical user interface (GUI);
(d) receive, via the interactive GUI, a user selection of a final hotspot set comprising at least a portion of the one or more automatically detected hotspots; and
(e) store and/or provide, for display and/or further processing, the final hotspot set.

91. The system of claim 90, wherein the instructions cause the processor to:
(f) receive, via the GUI, a user selection of one or more additional, user-identified, hotspots for inclusion in the final hotspot set; and
(g) update, the final hotspot set to include the one or more additional user-identified hotspots.

92. The system of either claim 90 or 91, wherein at step (b) the instructions cause the processor to use one or more machine learning modules.

93. A system for automatically processing 3D images of a subject to identify and characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) automatically detect one or more hotspots within the 3D functional image, each hotspot corresponding to a local region of elevated intensity with respect to its surrounding and representing a potential cancerous lesion within the subject;
(c) automatically determine, for each of at least a portion of the one or more hotspots, an anatomical classification corresponding to a particular anatomical region and/or group of anatomical regions within the subject in which the potential cancerous lesion that the hotspot represents is determined to be located; and
(d) store and/or provide, for display and/or further processing, an identification of the one or more hotspots along with, for each hotspot, the anatomical classification corresponding to the hotspot.

94. The system of claim 93, wherein the instructions cause the processor to perform step (b) using one or more machine learning modules.

95. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) receiving, by the processor, a 3D anatomical image of the subject obtained using an anatomical imaging modality;
(c) receiving, by the processor, a 3D segmentation map identifying one or more particular tissue region(s) or group(s) of tissue regions within the 3D functional image and/or within the 3D anatomical image;
(d) automatically detecting and/or segmenting, by the processor, using one or more machine learning module(s), a set of one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image,
wherein at least one of the one or more machine learning module(s) receives, as input (i) the 3D functional image, (ii) the 3D anatomical image, and (iii) the 3D segmentation map; and
(e) storing and/or providing, for display and/or further processing, the hotspot list and/or the 3D hotspot map.

96. The method of claim 95, comprising:
receiving, by the processor, an initial 3D segmentation map that identifies one or more particular tissue regions within the 3D anatomical image and/or the 3D functional image; and
identifying, by the processor, at least a portion of the one or more particular tissue regions as belonging to a particular one of one or more tissue grouping(s) and updating, by the processor, the 3D segmentation map to indicate the identified particular regions as belonging to the particular tissue grouping; and
using, by the processor, the updated 3D segmentation map as input to at least one of the one or more machine learning modules.

97. The method of claim 96, wherein the one or more tissue groupings comprise a soft-tissue grouping, such that particular tissue regions that represent soft-tissue are identified as belonging to the soft-tissue grouping.

98. The method of claim 96 or 97, wherein the one or more tissue groupings comprise a bone tissue grouping, such that particular tissue regions that represent bone are identified as belonging to the bone tissue grouping.

99. The method of any one of claims 96 to 98, wherein the one or more tissue groupings comprise a high-uptake organ grouping, such that one or more organs associated with high radiopharmaceutical uptake are identified as belonging to the high uptake grouping.

100. The method of any one of claims 95 to 99, comprising, for each detected and/or segmented hotspot, determining, by the processor, a classification for the hotspot.

101. The method of claim 100, comprising using at least one of the one or more machine learning modules to determine, for each detected and/or segmented lesion, the classification for the hotspot.

102. The method of any one of claims 95 to 101, wherein the one or more machine learning modules comprise:
(A) a full body lesion detection module that detects and/or segments hotspots throughout an entire body; and
(B) a prostate lesion module that detects and/or segments hotspots within the prostate.

103. The method of claim 102, comprising generating hotspot list and/or maps using each of (A) and (B) and merging the results.

104. The method of any one of claims 95 to 103, wherein:
step (d) comprises:
segmenting and classifying the set of one or more hotspots to create a labeled 3D hotspot map that identifies, for each hotspot, a corresponding 3D hotspot volume within the 3D functional image and in which each hotspot volume is labeled as belonging to a particular hotspot class of a plurality of hotspot classes by:
using a first machine learning module to segment a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class;
using a second machine learning module to segment a second initial set of one or more hotspots within the 3D functional image, thereby creating a second initial 3D hotspot map that identifies a second set of initial hotspot volumes, wherein the second machine learning module segments the 3D functional image to according to the plurality of different hotspot classes, such that the second initial 3D hotspot map is a multi-class 3D hotspot map in which each hotspot volume is labeled as belonging to a particular one of the plurality of different hotspot classes; and
merging, by the processor, the first initial 3D hotspot map and the second initial 3D hotspot map by, for at least a portion of the hotspot volumes identified by the first initial 3D hotspot map:
identifying a matching hotspot volume of the second initial 3D hotspot map, the matching hotspot volume of the second 3D hotspot map having been labeled as belonging to a particular hotspot class of the plurality of different hotspot classes; and
labeling the particular hotspot volume of the first initial 3D hotspot map as belonging to the particular hotspot class, thereby creating a merged 3D hotspot map that includes segmented hotspot volumes of the first 3D hotspot map having been labeled according classes that matching hotspot volumes of the second 3D hotspot map are identified as belonging to; and
step (e) comprises storing and/or providing, for display and/or further processing, the merged 3D hotspot map.

105. The method of claim 104, wherein the plurality of different hotspot classes comprise one or more members selected from the group consisting of:
(i) bone hotspots, determined to represent lesions located in bone,
(ii) lymph hotspots, determined to represent lesions located in lymph nodes, and
(iii) prostate hotspots, determined to represent lesions located in a prostate.

106. The method of any one of claims 95 to 105, further comprising:
(f) receiving and/or accessing the hotspot list; and
(g) for each hotspot in the hotspot list, segmenting the hotspot using an analytical model.

107. The method of any one of claims 95 to 105, further comprising:
(h) receiving and/or accessing the hotspot map; and
(i) for each hotspot in the hotspot map, segmenting the hotspot using an analytical model.

108. The method of claim 107, wherein the analytical model is an adaptive thresholding method, and step (i) comprises:
determining one or more reference values, each based on a measure of intensities of voxels of the 3D functional image located within a particular reference volume corresponding to a particular reference tissue region; and
for each particular hotspot volume of the 3D hotspot map:
determining, by the processor, a corresponding hotspot intensity based on intensities of voxels within the particular hotspot volume; and
determining, by the processor, a hotspot-specific threshold value for the particular hotspot based on (i) the corresponding hotspot intensity and (ii) at least one of the one or more reference value(s).

109. The method of claim 108, wherein the hotspot-specific threshold value is determined using a particular threshold function selected from a plurality of threshold functions, the particular threshold function selected based a comparison of the corresponding hotspot intensity with the at least one reference value.

110. The method of claim 108 or 109, wherein the hotspot-specific threshold value is determined as a variable percentage of the corresponding hotspot intensity, wherein the variable percentage decreases with increasing hotspot intensity.

111. A method for automatically processing 3D images of a subject to identify and/or characterize cancerous lesions within the subject, the method comprising:
(a) receiving, by a processor of a computing device, a 3D functional image of the subject obtained using a functional imaging modality;
(b) automatically segmenting, by the processor, using a first machine learning module, a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class;
(c) automatically segmenting, by the processor, using a second machine learning module, a second initial set of one or more hotspots within the 3D functional image, thereby creating a second initial 3D hotspot map that identifies a second set of initial hotspot volumes, wherein the second machine learning module segments the 3D functional image to according to a plurality of different hotspot classes, such that the second initial 3D hotspot map is a multi-class 3D hotspot map in which each hotspot volume is labeled as belonging to a particular one of the plurality of different hotspot classes;
(d) merging, by the processor, the first initial 3D hotspot map and the second initial 3D hotspot map by, for each particular hotspot volume of at least a portion of the first set of initial hotspot volumes identified by the first initial 3D hotspot map:
identifying a matching hotspot volume of the second initial 3D hotspot map, the matching hotspot volume of the second 3D hotspot map having been labeled as belonging to a particular hotspot class of the plurality of different hotspot classes; and
labeling the particular hotspot volume of the first initial 3D hotspot map as belonging to the particular hotspot class, thereby creating a merged 3D hotspot map that includes segmented hotspot volumes of the first 3D hotspot map having been labeled according classes that matching hotspots of the second 3D hotspot map are identified as belonging to; and
(e) storing and/or providing, for display and/or further processing, the merged 3D hotspot map.

112. The method of claim 111, wherein the plurality of different hotspot classes comprises one or more members selected from the group consisting of:
(i) bone hotspots, determined to represent lesions located in bone,
(ii) lymph hotspots, determined to represent lesions located in lymph nodes, and
(iii) prostate hotspots, determined to represent lesions located in a prostate.

113. A method for automatically processing 3D images of a subject to identify and/or characterize cancerous lesions within the subject, via an adaptive thresholding approach the method comprising:
(a) receiving, by a processor of a computing device, a 3D functional image of the subject obtained using a functional imaging modality;
(b) receiving, by the processor, a preliminary 3D hotspot map identifying, within the 3D functional image, one or more preliminary hotspot volumes;
(c) determining, by the processor, one or more reference values, each based on a measure of intensities of voxels of the 3D functional image located within a particular reference volume corresponding to a particular reference tissue region;
(d) creating, by the processor, a refined 3D hotspot map based on the preliminary hotspot volumes and using an adaptive threshold-based segmentation by, for each particular preliminary hotspot volume of at least a portion of the one or more preliminary hotspot volumes identified by the preliminary 3D hotspot map:
determining a corresponding hotspot intensity based on intensities of voxels within the particular preliminary hotspot volume;
determining a hotspot-specific threshold value for the particular preliminary hotspot volume based on (i) the corresponding hotspot intensity and (ii) at least one of the one or more reference value(s);
segmenting at least a portion of the 3D functional using a threshold-based segmentation algorithm that performs image segmentation using the hotspot-specific threshold value determined for the particular preliminary hotspot volume, thereby determining a refined, analytically segmented, hotspot volume corresponding to the particular preliminary hotspot volume; and
including the refined hotspot volume in the refined 3D hotspot map; and
(e) storing and/or providing, for display and/or further processing, the refined 3D hotspot map.

114. The method of claim 113, wherein the hotspot-specific threshold value is determined using a particular threshold function selected from a plurality of threshold functions, the particular threshold function selected based a comparison of the corresponding hotspot intensity with the at least one reference value.

115. The method of claim 113 or 114, wherein the hotspot-specific threshold value is determined as a variable percentage of the corresponding hotspot intensity, wherein the variable percentage decreases with increasing hotspot intensity.

116. A method for automatically processing 3D images of a subject to identify and/or characterize cancerous lesions within the subject, the method comprising:
(a) receiving, by a processor of a computing device, a 3D anatomical image of the subject obtained using an anatomical imaging modality, wherein the 3D anatomical image comprises a graphical representation of tissue within the subject;
(b) automatically segmenting, by the processor, the 3D anatomical image to create a 3D segmentation map that identifies a plurality of volumes of interest (VOIs) in the 3D anatomical image, including a liver volume corresponding to a liver of the subject and an aorta volume corresponding to an aorta portion;
(c) receiving, by the processor, a 3D functional image of the subject obtained using a functional imaging modality;
(d) automatically segmenting, by the processor, one or more hotspots within the 3D functional image, each segmented hotspot corresponding to a local region of elevated intensity with respect to its surrounding and representing a potential cancerous lesion within the subject, thereby identifying one or more automatically segmented hotspot volumes;
(e) causing, by the processor, rendering of a graphical representation of the one or more automatically segmented hotspot volumes for display within an interactive graphical user interface (GUI);
(f) receiving, by the processor, via the interactive GUI, a user selection of a final hotspot set comprising at least a portion of the one or more automatically segmented hotspot volumes;
(g) determining, by the processor, for each hotspot volume of the final set, a lesion index value based on (i) intensities of voxels of the functional image corresponding to the hotspot volume and (ii) one or more reference values determined using intensities of voxels of the functional image corresponding to the liver volume and the aorta volume; and
(e) storing and/or providing for display and/or further processing, the final hotspot set and/or lesion index values.

117. The method of claim 116, wherein:
step (b) comprises segmenting the anatomical image such that the 3D segmentation map identifies one or more bone volumes corresponding to one or more bones of the subject, and
step (d) comprises identifying, within the functional image, a skeletal volume using the one or more bone volumes and segmenting one or more bone hotspot volumes located within the skeletal volume.

118. The method of claim 116 or claim 117, wherein:
step (b) comprises segmenting the anatomical image such that the 3D segmentation map identifies one or more organ volumes corresponding to soft-tissue organs of the subject, and
step (d) comprises identifying, within the functional image, one or more soft tissue volumes using the one or more segmented organ volumes and segmenting one or more lymph and/or prostate hotspot volumes located within the soft tissue volume.

119. The method of claim 118, wherein step (d) further comprises, prior to segmenting the one or more lymph and/or prostate hotspot volumes, adjusting intensities of the functional image to suppress intensity from one or more high-uptake tissue regions.

120. The method of any one of claims 116 to 119, wherein step (g) comprises determining a liver reference value using intensities of voxels of the functional image corresponding to the liver volume.

121. The method of claim 120, comprising fitting a two component Gaussian mixture model two a histogram of intensities of functional image voxels corresponding to the liver volume, using the two-component Gaussian mixture model fit to identify and exclude voxels having intensities associated with regions of abnormally low uptake from the liver volume, and determining the liver reference value using intensities of remaining voxels.

122. A system for automatically processing 3D images of a subject to identify and/or characterize cancerous lesions within the subject, the system comprising:
a processor of a computing device; and
a memory having instruction stored thereon, wherein the instructions, when executed by the processor, cause the processor to:
(a) receive a 3D functional image of the subject obtained using a functional imaging modality;
(b) receive a 3D anatomical image of the subject obtained using an anatomical imaging modality;
(c) receive a 3D segmentation map identifying one or more particular tissue region(s) or group(s) of tissue regions within the 3D functional image and/or within the 3D anatomical image;
(d) automatically detect and/or segment, using one or more machine learning module(s), a set of one or more hotspots within the 3D functional image, each hotspot corresponding to a local region of elevated intensity with respect to its surrounding and representing a potential cancerous lesion within the subject, thereby creating one or both of (i) and (ii) as follows: (i) a hotspot list identifying, for each hotspot, a location of the hotspot, and (ii) a 3D hotspot map, identifying, for each hotspot, a corresponding 3D hotspot volume within the 3D functional image,
wherein at least one of the one or more machine learning module(s) receives, as input (i) the 3D functional image, (ii) the 3D anatomical image, and (iii) the 3D segmentation map; and
(e) store and/or provide, for display and/or further processing, the hotspot list and/or the 3D hotspot map.

123. The system of claim 122, wherein the instructions cause the processor to:
receive an initial 3D segmentation map that identifies one or more particular tissue regions within the 3D anatomical image and/or the 3D functional image;
identify at least a portion of the one or more particular tissue regions as belonging to a particular one of one or more tissue groupings and update the 3D segmentation map to indicate the identified particular regions as belonging to the particular tissue grouping; and
use the updated 3D segmentation map as input to at least one of the one or more machine learning modules.

124. The system of claim 123, wherein the one or more tissue groupings comprise a soft-tissue grouping, such that particular tissue regions that represent soft-tissue are identified as belonging to the soft-tissue grouping.

125. The system of claim 123 or 124, wherein the one or more tissue groupings comprise a bone tissue grouping, such that particular tissue regions that represent bone are identified as belonging to the bone tissue grouping.

126. The system of any one of claims 123 to 125, wherein the one or more tissue groupings comprise a high-uptake organ grouping, such that one or more organs associated with high radiopharmaceutical uptake are identified as belonging to the high uptake grouping.

127. The system of any one of claims 122 to 126, wherein the instructions cause the processor to, for each detected and/or segmented hotspot, determine a classification for the hotspot.

128. The system of claim 127, wherein the instructions cause the processor to use at least one of the one or more machine learning modules to determine, for each detected and/or segmented hotspot, the classification for the hotspot.

129. The system of any one of claims 122 to 128, wherein the one or more machine learning modules comprise:
(A) a full body lesion detection module that detects and/or segments hotspots throughout an entire body; and
(B) a prostate lesion module that detects and/or segments hotspots within the prostate.

130. The system of claim 129, wherein the instructions cause the processor to generate the hotspot list and/or maps using each of (A) and (B) and merge the results.

131. The system of any one of claims 122 to 130, wherein:
at step (d) the instructions cause the processor to segment and classify the set of one or more hotspots to create a labeled 3D hotspot map that identifies, for each hotspot, a corresponding 3D hotspot volume within the 3D functional image, and in which each hotspot is labeled as belonging to a particular hotspot class of a plurality of hotspot classes by:
using a first machine learning module to segment a first initial set of one or more hotspots within the 3D functional image, thereby creating a first initial 3D hotspot map that identifies a first set of initial hotspot volumes, wherein the first machine learning module segments hotspots of the 3D functional image according to a single hotspot class;
using a second machine learning module to segment a second initial set of one or more hotspots within the 3D functional image, thereby creating a second initial 3D hotspot map that identifies a second set of initial hotspot volumes, wherein the second machine learning module segments the 3D functional image to according to the plurality of different hotspot classes, such that the second initial 3D hotspot map is a multi-class 3D hotspot map in which each hotspot volume is labeled as belonging to a particular one of the plurality of different hotspot classes; and
merging the first initial 3D hotspot map and the second initial 3D hotspot map by, of at least a portion of the hotspot volumes identified by the first initial 3D hotspot map:
identifying a matching hotspot volume of the second initial 3D hotspot map, the matching hotspot volume of the second 3D hotspot map having been labeled as belonging to a particular hotspot class of the plurality of different hotspot classes; and
labeling the particular hotspot volume of the first initial 3D hotspot map as belonging to the particular hotspot class, thereby creating a merged 3D hotspot map that includes segmented hotspot volumes of the first 3D hotspot map having been labeled according classes that matching hotspots of the second 3D hotspot map are identified as belonging to; and
at step (e) the instructions cause the processor to store and/or provide, for display and/or further processing, the merged 3D hotspot map.

132. The system of claim 131, wherein the plurality of different hotspot classes comprise one or more members selected from the group consisting of:
(i) bone hotspots, determined to represent lesions located in bone,
(ii) lymph hotspots, determined to represent lesions located in lymph nodes, and
(iii) prostate hotspots, determined to represent lesions located in a prostate.

133. The system of any one of claims 122 to 132, wherein the instructions further cause the processor to:
(f) receive and/or access the hotspot list; and
(g) for each hotspot in the hotspot list, segment the hotspot using an analytical model.

134. The system of any one of claims 122 to 133, wherein the instructions further cause the processor to:
(h) receive and/or access the hotspot map;
(i) for each hotspot in the hotspot map, segment the hotspot using an analytical model.

135. The system of claim 134, wherein the analytical model is an adaptive thresholding method, and at step (i), the instructions cause the processor to:
determine one or more reference values, each based on a measure of intensities of voxels of the 3D functional image located within a particular reference volume corresponding to a particular reference tissue region; and
for each particular hotspot volume of the 3D hotspot map:
determine a corresponding hotspot intensity based on intensities of voxels within the particular hotspot volume; and
determine a hotspot-specific threshold value for the particular hotspot based on (i) the corresponding hotspot intensity and (ii) at least one of the one or more reference value(s).

136. The system of claim 135, wherein the hotspot-specific threshold value is determined using a particular threshold function selected from a plurality of threshold functions, the particular threshold function selected based a comparison of the corresponding hotspot intensity with the at least one reference value.

137. The system of claim 135 or 136, wherein the hotspot-specific threshold value is determined as a variable percentage of the corresponding hotspot intensity, wherein the variable percentage decreases with increasing hotspot intensity.

138. A system for automatically processing 3D images of a subject to identify and/or characterize cancerous lesions within the subject, the system comprising:
a processor of a computing device; and
a memory having instructions stored thereon, wherein the instructions, when executed by the processor, cause the processor to:
(a) receive a 3D functional image of the subject obtained using a functional imaging modality;
(b) automatically segment, using a first machine learning module, a first initial set of one or more hotspots within the 3D functional image, thereby creating a first initial 3D hotspot map that identifies a first set of initial hotspot volumes, a corresponding 3D hotspot volume within the 3D functional image, wherein the first machine learning module segments hotspots of the 3D functional image according to a single hotspot class;
(c) automatically segment, using a second machine learning module, a second initial set of one or more hotspots within the 3D functional image, thereby creating a second initial 3D hotspot map that identifies a second set of initial hotspot volumes, wherein the second machine learning module segments the 3D functional image to according to a plurality of different hotspot classes, such that the second initial 3D hotspot map is a multi-class 3D hotspot map in which each hotspot volume is labeled as belonging to a particular one of the plurality of different hotspot classes;
(d) merge the first initial 3D hotspot map and the second initial 3D hotspot map by, for each particular hotspot volume of at least a portion of the first set of initial hotspot volumes identified by the first initial 3D hotspot map:
identifying a matching hotspot volume of the second initial 3D hotspot map, the matching hotspot volume of the second 3D hotspot map having been labeled as belonging to a particular hotspot class of the plurality of different hotspot classes; and
labeling the particular hotspot volume of the first initial 3D hotspot map as belonging to the particular hotspot class, thereby creating a merged 3D hotspot map that includes segmented hotspot volumes of the first 3D hotspot map having been labeled according classes that matching hotspots of the second 3D hotspot map are identified as belonging to; and
(e) store and/or provide, for display and/or further processing, the merged 3D hotspot map.

139. The system of claim 138, wherein the plurality of different hotspot classes comprises one or more members selected from the group consisting of:
(i) bone hotspots, determined to represent lesions located in bone,
(ii) lymph hotspots, determined to represent lesions located in lymph nodes, and
(i) prostate hotspots, determined to represent lesions located in a prostate.

140. A system for automatically processing 3D images of a subject to identify and/or characterize cancerous lesions within the subject, via an adaptive thresholding approach the system comprising:
a processor of a computing device; and
a memory having instructions stored thereon, wherein the instructions, when executed by the processor, cause the processor to:
(a) receive a 3D functional image of the subject obtained using a functional imaging modality;
(b) receive a preliminary 3D hotspot map identifying, within the 3D functional image, one or more preliminary hotspot volumes;
(c) determine one or more reference values, each based on a measure of intensities of voxels of the 3D functional image located within a particular reference volume corresponding to a particular reference tissue region;
(d) create a refined 3D hotspot map based on the preliminary hotspot volumes and using an adaptive threshold-based segmentation by, for each particular preliminary hotspot volume of at least a portion of the one or more preliminary hotspot volumes identified by the preliminary 3D hotspot map:
determining a corresponding hotspot intensity based on intensities of voxels within the particular preliminary hotspot volume; and
determining a hotspot-specific threshold value for the particular preliminary hotspot based on (i) the corresponding hotspot intensity and (ii) at least one of the one or more reference value(s);
segmenting at least a portion of the 3D functional using a threshold-based segmentation algorithm that performs image segmentation using the hotspot-specific threshold value determined for the particular preliminary hotspot, thereby determining a refined, analytically segmented, hotspot volume corresponding to the particular preliminary hotspot volume; and
including the refined hotspot volume in the refined 3D hotspot map; and
(e) store and/or provide, for display and/or further processing, the refined 3D hotspot map.

141. The system of claim 140, wherein the hotspot-specific threshold value is determined using a particular threshold function selected from a plurality of threshold functions, the particular threshold function selected based a comparison of the corresponding hotspot intensity with the at least one reference value.

142. The system of claim 140 or 141, wherein the hotspot-specific threshold value is determined as a variable percentage of the corresponding hotspot intensity, wherein the variable percentage decreases with increasing hotspot intensity.

143. A system for automatically processing 3D images of a subject to identify and/or characterize cancerous lesions within the subject, the system comprising:
a processor of a computing device;
a memory having instructions stored thereon, wherein the instructions, when executed by the processor, cause the processor to:
(a) receive a 3D anatomical image of the subject obtained using an anatomical imaging modality, wherein the 3D anatomical image comprises a graphical representation of tissue within the subject;
(b) automatically segment the 3D anatomical image to create a 3D segmentation map that identifies a plurality of volumes of interest (VOIs) in the 3D anatomical image, including a liver volume corresponding to a liver of the subject and an aorta volume corresponding to an aorta portion;
(c) receive a 3D functional image of the subject obtained using a functional imaging modality;
(d) automatically segment one or more hotspots within the 3D functional image, each segmented hotspot corresponding to a local region of elevated intensity with respect to its surrounding and representing a potential cancerous lesion within the subject, thereby identifying one or more automatically segmented hotspot volumes;
(e) causing rendering of a graphical representation of the one or more automatically segmented hotspot volumes for display within an interactive graphical user interface (GUI);
(f) receive, via the interactive GUI, a user selection of a final hotspot set comprising at least a portion of the one or more automatically segmented hotspot volumes;
(g) determine, for each hotspot volume of the final set, a lesion index value based on (i) intensities of voxels of the functional image corresponding to the hotspot volume and (ii) one or more reference values determined using intensities of voxels of the functional image corresponding to the liver volume and the aorta volume; and
(e) store and/or provide for display and/or further processing, the final hotspot set and/or lesion index values.

144. The system of claim 143, wherein:
at step (b) the instructions cause the processor to segment the anatomical image, such that the 3D segmentation map identifies one or more bone volumes corresponding to one or more bones of the subject, and
at step (d) the instructions cause the processor to identify, within the functional image, a skeletal volume using the one or more bone volumes and segmenting one or more bone hotspot volumes located within the skeletal volume.

145. The system of claim 143 or claim 144, wherein:
at step (b) the instructions cause the processor to segment the anatomical image such that the 3D segmentation map identifies one or more organ volumes corresponding to soft-tissue organs of the subject, and
at step (d) the instructions cause the processor to identify, within the functional image, a soft tissue volume using the one or more segmented organ volumes and segmenting one or more lymph and/or prostate hotspot volumes located within the soft tissue volume.

146. The system of claim 145, wherein at step (d) the instructions cause the processor to, prior to segmenting the one or more lymph and/or prostate hotspot volumes, adjust intensities of the functional image to suppress intensity from one or more high-uptake tissue regions.

147. The system of any one of claims 143 to 146, wherein at step (g) the instructions cause the processor to determine a liver reference value using intensities of voxels of the functional image corresponding to the liver volume.

148. The system of claim 147, wherein the instructions cause the processor to:
fit a two component Gaussian mixture model two a histogram of intensities of functional image voxels corresponding to the liver volume,
use the two-component Gaussian mixture model fit to identify and exclude voxels having intensities associated with regions of abnormally low uptake from the liver volume, and
determine the liver reference value using intensities of remaining voxels.
